SEQUENCE LISTING (203 sequences)

SEQ ID NO: 1 - protein, 217 aa, Artificial Sequence
  Light chain
QSVLTQPPSASGTPGQRVTISCTGSSSNIGAGYDVHWYQQLPGTAPKLLIYADDHRPSGVPDRFSGSKSGTSASLAISGLRSEDEADYYCASWDDSQRAVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 447 aa, Artificial Sequence
  Heavy chain
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 118 aa, Artificial Sequence
  1A01-VH
EVQLLESGGGLVQPGGSLRLSCAASGFTFSDYYMNWIRQTPGKGLEWVSLIGWDGGSTYYADSVKGRFTISRDNSENTLYLQMNSLRAEDTAVYYCARAYSGYELDYWGQGTLVTVSS

SEQ ID NO: 4 - protein, 117 aa, Artificial Sequence
  1B07-VH
EVQLLESGGGLVQPGGSLRLSCAASGFTFSSYGMHWVRQAPGKGLEWVAFTRYDGSNKYYADSVRGRFTISRDNSKNTLYLQMNSLRAEDTAVYYCARENIDAFDVWGQGTLVTVSS

SEQ ID NO: 5 - protein, 122 aa, Artificial Sequence
  1C04-VH
EVQLLESGGGLVQPGGSLRLSCAASGFTFSSYAMSWVRQAPGKGLEWVSSISDSGAGRYYADSVEGRFTISRDNSKNTLYLQMNSLRAEDTAVYYCARTHDSGELLDAFDIWGQGTLVTVSS

SEQ ID NO: 6 - protein, 124 aa, Artificial Sequence
  1E05-VH
EVQLLESGGGLVQPGGSLRLSCAASGFTFSTYAMNWVRQVPGKGLEWVAVISYDGSNKNYVDSVKGRFTISRDNSKNTLYLQMNSLRAEDTAVYYCARNFDNSGYAIPDAFDIWGQGTLVTVSS

SEQ ID NO: 7 - protein, 121 aa, Artificial Sequence
  2A09-VH
EVQLLESGGGLVQPGGSLRLSCAASGFTFSNAWMSWVRQAPGKGLEWVAYISRDADITHYPASVKGRFTISRDNSKNTLYLQMNSLRAEDTAVYYCTTGFDYAGDDAFDIWGQGTLVTVSS

SEQ ID NO: 8 - protein, 119 aa, Artificial Sequence
  2B08-VH
EVQLLESGGGLVQPGGSLRLSCAASGFTFSDYYMSWVRQAPGKGLEWVALIGHDGNNKYYLDSLEGRFTISRDNSKNTLYLQMNSLRAEDTAVYYCARATDSGYDLLYWGQGTLVTVSS

SEQ ID NO: 9 - protein, 117 aa, Artificial Sequence
  2E08-VH
EVQLLESGGGLVQPGGSLRLSCAASGFTFSDYYMSWIRQAPGKGLEWVSAIGFSDDNTYYADSVKGRFTISRDNSKNTLYLQMNSLRAEDTAVYYCAGGDGSGWSFWGQGTLVTVSS

SEQ ID NO: 10 - protein, 117 aa, Artificial Sequence
  5C04-VH
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAREWRDAFDIWGQGTLVTVSS

SEQ ID NO: 11 - protein, 117 aa, Artificial Sequence
  5C05-VH
EVQLLESGGGLVQPGGSLRLSCAASGFTFSTYGMHWVRQAPGKGLEWVAVISYDGSNKYYADSVKGRFTISRDNSKNTLYLQMNSLRAEDTAVYYCARENFDAFDVWGQGTLVTVSS

SEQ ID NO: 12 - protein, 117 aa, Artificial Sequence
  5D07-VH
EVQLLESGGGLVQPGGSLRLSCAASGFTFSTYGMHWVRQAPGKGLEWVAVIAYDGSKKDYADSVKGRFTISRDNSKNTLYLQMNSLRAEDTAVYYCAREYRDAFDIWGQGTLVTVSS

SEQ ID NO: 13 - protein, 117 aa, Artificial Sequence
  5E12-VH
EVQLLESGGGLVQPGGSLRLSCAASGFTFSSYGMHWVRQAPGKGLEWVAVISYDGINKDYADSMKGRFTISRDNSKNTLYLQMNSLRAEDTAVYYCARERKDAFDIWGQGTLVTVSS

SEQ ID NO: 14 - protein, 117 aa, Artificial Sequence
  5G08-VH
EVQLLESGGGLVQPGGSLRLSCAASGFTFNNYGMHWVRQAPGKGLEWVAVISYDGSNRYYADSVKGRFTMSRDNSKNTLYLQMNSLRAEDTAVYYCARDRWNGMDVWGQGTLVTVSS

SEQ ID NO: 15 - protein, 119 aa, Artificial Sequence
  5H06-VH
EVQLLESGGGLVQPGGSLRLSCAASGFTFSSYGMHWVRQAPGKGLEWVAVISYDGSDTAYADSVKGRFTISRDNSKNTLYLQMNSLRAEDTAVYYCARDHSVIGAFDIWGQGTLVTVSS

SEQ ID NO: 16 - protein, 119 aa, Artificial Sequence
  6A09-VH
EVQLLESGGGLVQPGGSLRLSCAASGFTFSSYGMHWVRQAPGKGLEWVAVTSYDGNTKYYANSVKGRFTISRDNSKNTLYLQMNSLRAEDTAVYYCAREDCGGDCFDYWGQGTLVTVSS

SEQ ID NO: 17 - protein, 118 aa, Artificial Sequence
  6B01-VH
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCARDQLGEAFDIWGQGTLVTVSS

SEQ ID NO: 18 - protein, 117 aa, Artificial Sequence
  6C11-VH
EVQLLESGGGLVQPGGSLRLSCAASGFTFDDYGMSWVRQAPGKGLEWVSAISGSGSSTYYADSVKGRFTISRDNSKNTLYLQMNSLRAEDTAVYYCAGGDIDYFDYWGQGTLVTVSS

SEQ ID NO: 19 - protein, 117 aa, Artificial Sequence
  6C12-VH
EVQLLESGGGLVQPGGSLRLSCAASGFTFSSYGMHWVRQAPGKGLEWVAVISYDGSNKYYADSVKGRFTISRDNSKNTLYLQMNSLRAEDTAVYYCARERRDAFDIWGQGTLVTVSS

SEQ ID NO: 20 - protein, 119 aa, Artificial Sequence
  6D01-VH
EVQLLESGGGLVQPGGSLRLSCAASGFTFSSYGMHWVRQAPGKGLEWVAVISYDGSNKYYADSVKGRFTISRDNSKNTLYLQMNSLRAEDTAMYYCARDHSAAGYFDYWGQGTLVTVSS

SEQ ID NO: 21 - protein, 119 aa, Artificial Sequence
  6G03-VH
EVQLLESGGGLVQPGGSLRLSCAASGFTFGSYGMHWVRQAPGKGLEWVSGISWDSAIIDYAGSVKGRFTISRDNSKNTLYLQMNSLRAEDTAVYYCAKDEAAAGAFDIWGQGTLVTVSS

SEQ ID NO: 22 - protein, 121 aa, Artificial Sequence
  6G08-VH
EVQLLESGGGLVQPGGSLRLSCAASGFTLSSYGISWVRQAPGKGLEWVSGISGSGGNTYYADSVKGRFTISRDNSKNTLYLQMNSLRAEDTAVYYCASSVGAYANDAFDIWGQGTLVTVSS

SEQ ID NO: 23 - protein, 117 aa, Artificial Sequence
  6G11-VH
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSS

SEQ ID NO: 24 - protein, 117 aa, Artificial Sequence
  6H08-VH
EVQLLESGGGLVQPGGSLRLSCAASGFTFNNYGMHWVRQAPGKGLEWVAVISYDGSNKYYADSVKGRFTISKDNSKNTLYLQMNSLRAEDTAVYYCAREYKDAFDIWGQGTLVTVSS

SEQ ID NO: 25 - protein, 118 aa, Artificial Sequence
  7C07-VH
EVQLLESGGGLVQPGGSLRLSCAASGFTFSSYGMHWVRQAPGKGLEWVAVISYDGSNKYYADSVKGRFTISRDNSQNTLYLQMNSLRAEDTAVYYCAREFGYIILDYWGQGTLVTVSS

SEQ ID NO: 26 - protein, 117 aa, Artificial Sequence
  4B02-VH
EVQLLESGGGLVQPGGSLRLSCAASGFTFSNHGMHWVRQAPGKGLEWVAVISYDGTNKYYADSVRGRFTISRDNSKNTLYLQMNSLRAEDTAVYYCARETWDAFDVWGQGTLVTVSS

SEQ ID NO: 27 - protein, 111 aa, Artificial Sequence
  1A01-VL
QSVLTQPPSASGTPGQRVTISCSGSSSNIGNNAVNWYQQLPGTAPKLLIYDNNNRPSGVPDRFSGSKSGTSASLAISGLRSEDEADYYCAAWDDSLNASIFGGGTKLTVLG

SEQ ID NO: 28 - protein, 111 aa, Artificial Sequence
  1B07-VL
QSVLTQPPSASGTPGQRVTISCSGSSSNIGNNAVNWYQQLPGTAPKLLIYDNQQRPSGVPDRFSGSKSGTSASLAISGLRSEDEADYYCEAWDDRLFGPVFGGGTKLTVLG

SEQ ID NO: 29 - protein, 111 aa, Artificial Sequence
  1C04-VL
QSVLTQPPSASGTPGQRVTISCSGSSSNIGSNHVLWYQQLPGTAPKLLIYGNSNRPSGVPDRFSGSKSGTSASLAISGLRSEDEADYYCAAWDDSLNGWVFGGGTKLTVLG

SEQ ID NO: 30 - protein, 112 aa, Artificial Sequence
  1E05-VL
QSVLTQPPSASGTPGQRVTISCTGSSSNIGAGYDVHWYQQLPGTAPKLLIYDNNSRPSGVPDRFSGSKSGTSASLAISGLRSEDEADYYCAAWDDSLGGPVFGGGTKLTVLG

SEQ ID NO: 31 - protein, 112 aa, Artificial Sequence
  2A09-VL
QSVLTQPPSASGTPGQRVTISCSGSSSNIGSNAVNWYQQLPGTAPKLLIYGNSDRPSGVPDRFSGSKSGTSASLAISGLRSEDEADYYCAAWDDSLNGRWVFGGGTKLTVLG

SEQ ID NO: 32 - protein, 111 aa, Artificial Sequence
  2B08-VL
QSVLTQPPSASGTPGQRVTISCSGSSSNIGNNAVNWYQQLPGTAPKLLIYYDDLLPSGVPDRFSGSKSGTSASLAISGLRSEDEADYYCTTWDDSLSGVVFGGGTKLTVLG

SEQ ID NO: 33 - protein, 111 aa, Artificial Sequence
  2E08-VL
QSVLTQPPSASGTPGQRVTISCSGSSSNIGNNAVNWYQQLPGTAPKLLIYDNNKRPSGVPDRFSGSKSGTSASLAISGLRSEDEADYYCATWDDSLRGWVFGGGTKLTVLG

SEQ ID NO: 34 - protein, 113 aa, Artificial Sequence
  5C04-VL
QSVLTQPPSASGTPGQRVTISCTGSSSNIGAGYDVHWYQQLPGTAPKLLIYSDNQRPSGVPDRFSGSKSGTSASLAISGLRSEDEADYYCAAWDDSLSGSWVFGGGTKLTVLG

SEQ ID NO: 35 - protein, 113 aa, Artificial Sequence
  5C05-VL
QSVLTQPPSASGTPGQRVTISCTGSSSNIGAGYDVHWYQQLPGTAPKLLIYSNSQRPSGVPDRFSGSKSGTSASLAISGLRSEDEADYYCAAWDDSLNGQVVFGGGTKLTVLG

SEQ ID NO: 36 - protein, 112 aa, Artificial Sequence
  5D07-VL
QSVLTQPPSASGTPGQRVTISCTGSSSNIGAGYDVHWYQQLPGTAPKLLIYGNSNRPSGVPDRFSGSKSGTTASLAISGLRSEDEADYYCAAWDDSVSGWMFGGGTKLTVLG

SEQ ID NO: 37 - protein, 112 aa, Artificial Sequence
  5E12-VL
QSVLTQPPSASGTPGQRVTISCTGSSSNIGAGYDVHWYQQLPGTAPKLLIYSNNQRPSGVPDRFSGSKSGTSASLAISGLRSEDEADYYCATWDDSLNGLVFGGGTKLTVLG

SEQ ID NO: 38 - protein, 113 aa, Artificial Sequence
  5G08-VL
QSVLTQPPSASGTPGQRVTISCSGSSSNIGAGYDVHWYQQLPGTAPKLLIYANNQRPSGVPDRFSGSKSGTSASLAISGLRSEDEADYYCAAWDDSLNGPWVFGGGTKLTVLG

SEQ ID NO: 39 - protein, 110 aa, Artificial Sequence
  5H06-VL
QSVLTQPPSASGTPGQRVTISCSGSSSNIGSNTVNWYQQLPGTAPKLLIYDNNKRPSGVPDRFSGSKSGTSASLAISGLRSEDEADYYCSSYAGSNNVVFGGGTKLTVLG

SEQ ID NO: 40 - protein, 112 aa, Artificial Sequence
  6A09-VL
QSVLTQPPSASGTPGQRVTISCTGSSSNIGAGYDVHWYQQLPGTAPKLLIYGNSNRPSGVPDRFSGSKSGTSASLAISGLRSEDEADYYCAAWDDSLNEGVFGGGTKLTVLG

SEQ ID NO: 41 - protein, 112 aa, Artificial Sequence
  6B01-VL
QSVLTQPPSASGTPGQRVTISCTGSSSNIGAGYDVHWYQQLPGTAPKLLIYDNNKRPSGVPDRFSGSKSGTSASLAISGLRSEDEADYYCATWDDSLSGPVFGGGTKLTVLG

SEQ ID NO: 42 - protein, 112 aa, Artificial Sequence
  6C11-VL
QSVLTQPPSASGTPGQRVTISCTGSSSNFGAGYDVHWYQQLPGTAPKLLIYENNKRPSGVPDRFSGSKSGTSASLAISGLRSEDEADYYCAAWDDSLNGPVFGGGTKLTVLG

SEQ ID NO: 43 - protein, 110 aa, Artificial Sequence
  6C12-VL
QSVLTQPPSASGTPGQRVTISCTGSSSNIGAGYDVHWYQQLPGTAPKLLIYSDNQRPSGVPDRFSGSKSGTSASLAISGLRSEDEADYYCATWDSDTPVFGGGTKLTVLG

SEQ ID NO: 44 - protein, 111 aa, Artificial Sequence
  6D01-VL
QSVLTQPPSASGTPGQRVTISCSGSSSNIGSNTVNWYQQLPGTAPKLLIYGNSIRPSGGPDRFSGSKSGTSASLAISGLRSEDEADYYCASWDDSLSSPVFGGGTKLTVLG

SEQ ID NO: 45 - protein, 113 aa, Artificial Sequence
  6G03-VL
QSVLTQPPSASGTPGQRVTISCTGSSSNIGAGYDVHWYQQLPGTAPKLLIYGNTDRPSGVPDRFSGSKSGTSASLAISGLRSEDEADYYCAAWDDSLSGPVVFGGGTKLTVLG

SEQ ID NO: 46 - protein, 112 aa, Artificial Sequence
  6G08-VL
QSVLTQPPSASGTPGQRVTISCTGSSSNIGAGYDVHWYQQLPGTAPKLLIYGDTNRPSGVPDRFSGSKSGTSASLAISGLRSEDEADYYCAAWDDSLNGPVFGGGTKLTVLG

SEQ ID NO: 47 - protein, 112 aa, Artificial Sequence
  6G11-VL
QSVLTQPPSASGTPGQRVTISCTGSSSNIGAGYDVHWYQQLPGTAPKLLIYADDHRPSGVPDRFSGSKSGTSASLAISGLRSEDEADYYCASWDDSQRAVIFGGGTKLTVLG

SEQ ID NO: 48 - protein, 109 aa, Artificial Sequence
  6H08-VL
QSVLTQPPSASGTPGQRVTISCTGSSSNIGSNTVNWYQQLPGTAPKLLIYDNNKRPSGVPDRFSGSKSGTSASLAISGLRSEDEADYYCQAWGTGIRVFGGGTKLTVLG

SEQ ID NO: 49 - protein, 111 aa, Artificial Sequence
  7C07-VL
QSVLTQPPSASGTPGQRVTISCSGSSSNIGSNTVNWYQQLPGTAPKLLIYRDYERPSGVPDRFSGSKSGTSASLAISGLRSEDEADYYCMAWDDSLSGVVFGGGTKLTVLG

SEQ ID NO: 50 - protein, 109 aa, Artificial Sequence
  4B02-VL
QSVLTQPPSASGTPGQRVTISCSGSSSNIGSNNANWYQQLPGTAPKLLIYDNNKRPSGVPDRFSGSKSGTSASLAISGLRSEDEADYYCQAWDSSTVVFGGGTKLTVLG

SEQ ID NO: 51 - protein, 5 aa, Artificial Sequence
  CDRH1
DYYMN

SEQ ID NO: 52 - protein, 17 aa, Artificial Sequence
  CDRH2
LIGWDGGSTYYADSVKG

SEQ ID NO: 53 - protein, 9 aa, Artificial Sequence
  CDRH3
AYSGYELDY

SEQ ID NO: 54 - protein, 13 aa, Artificial Sequence
  CDRL1
SGSSSNIGNNAVN

SEQ ID NO: 55 - protein, 7 aa, Artificial Sequence
  CDRL2
DNNNRPS

SEQ ID NO: 56 - protein, 11 aa, Artificial Sequence
  CDRL3
AAWDDSLNASI

SEQ ID NO: 57 - protein, 5 aa, Artificial Sequence
  CDRH1
SYGMH

SEQ ID NO: 58 - protein, 17 aa, Artificial Sequence
  CDRH2
FTRYDGSNKYYADSVRG

SEQ ID NO: 59 - protein, 8 aa, Artificial Sequence
  CDRH3
ENIDAFDV

SEQ ID NO: 60 - protein, 13 aa, Artificial Sequence
  CDRL1
SGSSSNIGNNAVN

SEQ ID NO: 61 - protein, 7 aa, Artificial Sequence
  CDRL2
DNQQRPS

SEQ ID NO: 62 - protein, 9 aa, Artificial Sequence
  CDRL3
WDDRLFGPV

SEQ ID NO: 63 - protein, 5 aa, Artificial Sequence
  CDRH1
SYAMS

SEQ ID NO: 64 - protein, 17 aa, Artificial Sequence
  CDRH2
SISDSGAGRYYADSVEG

SEQ ID NO: 65 - protein, 13 aa, Artificial Sequence
  CDRH3
THDSGELLDAFDI

SEQ ID NO: 66 - protein, 13 aa, Artificial Sequence
  CDRL1
SGSSSNIGSNHVL

SEQ ID NO: 67 - protein, 7 aa, Artificial Sequence
  CDRL2
GNSNRPS

SEQ ID NO: 68 - protein, 11 aa, Artificial Sequence
  CDRL3
AAWDDSLNGWV

SEQ ID NO: 69 - protein, 5 aa, Artificial Sequence
  CDRH1
TYAMN

SEQ ID NO: 70 - protein, 17 aa, Artificial Sequence
  CDRH2
VISYDGSNKNYVDSVKG

SEQ ID NO: 71 - protein, 15 aa, Artificial Sequence
  CDRH3
NFDNSGYAIPDAFDI

SEQ ID NO: 72 - protein, 14 aa, Artificial Sequence
  CDRL1
TGSSSNIGAGYDVH

SEQ ID NO: 73 - protein, 7 aa, Artificial Sequence
  CDRL2
DNNSRPS

SEQ ID NO: 74 - protein, 11 aa, Artificial Sequence
  CDRL3
AAWDDSLGGPV

SEQ ID NO: 75 - protein, 5 aa, Artificial Sequence
  CDRH1
NAWMS

SEQ ID NO: 76 - protein, 17 aa, Artificial Sequence
  CDRH2
YISRDADITHYPASVKG

SEQ ID NO: 77 - protein, 12 aa, Artificial Sequence
  CDRH3
GFDYAGDDAFDI

SEQ ID NO: 78 - protein, 13 aa, Artificial Sequence
  CDRL1
SGSSSNIGSNAVN

SEQ ID NO: 79 - protein, 7 aa, Artificial Sequence
  CDRL2
GNSDRPS

SEQ ID NO: 80 - protein, 12 aa, Artificial Sequence
  CDRL3
AAWDDSLNGRWV

SEQ ID NO: 81 - protein, 5 aa, Artificial Sequence
  CDRH1
DYYMS

SEQ ID NO: 82 - protein, 17 aa, Artificial Sequence
  CDRH2
LIGHDGNNKYYLDSLEG

SEQ ID NO: 83 - protein, 10 aa, Artificial Sequence
  CDRH3
ATDSGYDLLY

SEQ ID NO: 84 - protein, 13 aa, Artificial Sequence
  CDRL1
SGSSSNIGNNAVN

SEQ ID NO: 85 - protein, 7 aa, Artificial Sequence
  CDRL2
YDDLLPS

SEQ ID NO: 86 - protein, 11 aa, Artificial Sequence
  CDRL3
TTWDDSLSGVV

SEQ ID NO: 87 - protein, 5 aa, Artificial Sequence
  CDRH1
DYYMS

SEQ ID NO: 88 - protein, 17 aa, Artificial Sequence
  CDRH2
AIGFSDDNTYYADSVKG

SEQ ID NO: 89 - protein, 8 aa, Artificial Sequence
  CDRH3
GDGSGWSF

SEQ ID NO: 90 - protein, 13 aa, Artificial Sequence
  CDRL1
SGSSSNIGNNAVN

SEQ ID NO: 91 - protein, 7 aa, Artificial Sequence
  CDRL2
DNNKRPS

SEQ ID NO: 92 - protein, 11 aa, Artificial Sequence
  CDRL3
ATWDDSLRGWV

SEQ ID NO: 93 - protein, 5 aa, Artificial Sequence
  CDRH1
NYGMH

SEQ ID NO: 94 - protein, 17 aa, Artificial Sequence
  CDRH2
VISYDGSNKYYADSVKG

SEQ ID NO: 95 - protein, 7 aa, Artificial Sequence
  CDRH3
WRDAFDI

SEQ ID NO: 96 - protein, 14 aa, Artificial Sequence
  CDRL1
TGSSSNIGAGYDVH

SEQ ID NO: 97 - protein, 7 aa, Artificial Sequence
  CDRL2
SDNQRPS

SEQ ID NO: 98 - protein, 12 aa, Artificial Sequence
  CDRL3
AAWDDSLSGSWV

SEQ ID NO: 99 - protein, 5 aa, Artificial Sequence
  CDRH1
TYGMH

SEQ ID NO: 100 - protein, 17 aa, Artificial Sequence
  CDRH2
VISYDGSNKYYADSVKG

SEQ ID NO: 101 - protein, 8 aa, Artificial Sequence
  CDRH3
ENFDAFDV

SEQ ID NO: 102 - protein, 14 aa, Artificial Sequence
  CDRL1
TGSSSNIGAGYDVH

SEQ ID NO: 103 - protein, 7 aa, Artificial Sequence
  CDRL2
SNSQRPS

SEQ ID NO: 104 - protein, 12 aa, Artificial Sequence
  CDRL3
AAWDDSLNGQVV

SEQ ID NO: 105 - protein, 5 aa, Artificial Sequence
  CDRH1
TYGMH

SEQ ID NO: 106 - protein, 17 aa, Artificial Sequence
  CDRH2
VIAYDGSKKDYADSVKG

SEQ ID NO: 107 - protein, 8 aa, Artificial Sequence
  CDRH3
EYRDAFDI

SEQ ID NO: 108 - protein, 14 aa, Artificial Sequence
  CDRL1
TGSSSNIGAGYDVH

SEQ ID NO: 109 - protein, 7 aa, Artificial Sequence
  CDRL2
GNSNRPS

SEQ ID NO: 110 - protein, 11 aa, Artificial Sequence
  CDRL3
AAWDDSVSGWM

SEQ ID NO: 111 - protein, 5 aa, Artificial Sequence
  CDRH1
SYGMH

SEQ ID NO: 112 - protein, 17 aa, Artificial Sequence
  CDRH2
VISYDGINKDYADSMKG

SEQ ID NO: 113 - protein, 8 aa, Artificial Sequence
  CDRH3
ERKDAFDI

SEQ ID NO: 114 - protein, 14 aa, Artificial Sequence
  CDRL1
TGSSSNIGAGYDVH

SEQ ID NO: 115 - protein, 7 aa, Artificial Sequence
  CDRL2
SNNQRPS

SEQ ID NO: 116 - protein, 11 aa, Artificial Sequence
  CDRL3
ATWDDSLNGLV

SEQ ID NO: 117 - protein, 5 aa, Artificial Sequence
  CDRH1
NYGMH

SEQ ID NO: 118 - protein, 17 aa, Artificial Sequence
  CDRH2
VISYDGSNRYYADSVKG

SEQ ID NO: 119 - protein, 8 aa, Artificial Sequence
  CDRH3
DRWNGMDV

SEQ ID NO: 120 - protein, 14 aa, Artificial Sequence
  CDRL1
SGSSSNIGAGYDVH

SEQ ID NO: 121 - protein, 7 aa, Artificial Sequence
  CDRL2
ANNQRPS

SEQ ID NO: 122 - protein, 12 aa, Artificial Sequence
  CDRL3
AAWDDSLNGPWV

SEQ ID NO: 123 - protein, 5 aa, Artificial Sequence
  CDRH1
SYGMH

SEQ ID NO: 124 - protein, 17 aa, Artificial Sequence
  CDRH2
VISYDGSDTAYADSVKG

SEQ ID NO: 125 - protein, 10 aa, Artificial Sequence
  CDRH3
DHSVIGAFDI

SEQ ID NO: 126 - protein, 13 aa, Artificial Sequence
  CDRL1
SGSSSNIGSNTVN

SEQ ID NO: 127 - protein, 7 aa, Artificial Sequence
  CDRL2
DNNKRPS

SEQ ID NO: 128 - protein, 10 aa, Artificial Sequence
  CDRL3
SSYAGSNNVV

SEQ ID NO: 129 - protein, 5 aa, Artificial Sequence
  CDRH1
SYGMH

SEQ ID NO: 130 - protein, 17 aa, Artificial Sequence
  CDRH2
VTSYDGNTKYYANSVKG

SEQ ID NO: 131 - protein, 10 aa, Artificial Sequence
  CDRH3
EDCGGDCFDY

SEQ ID NO: 132 - protein, 14 aa, Artificial Sequence
  CDRL1
TGSSSNIGAGYDVH

SEQ ID NO: 133 - protein, 7 aa, Artificial Sequence
  CDRL2
GNSNRPS

SEQ ID NO: 134 - protein, 11 aa, Artificial Sequence
  CDRL3
AAWDDSLNEGV

SEQ ID NO: 135 - protein, 5 aa, Artificial Sequence
  CDRH1
NYGMH

SEQ ID NO: 136 - protein, 17 aa, Artificial Sequence
  CDRH2
VISYDGSNKYYADSVKG

SEQ ID NO: 137 - protein, 9 aa, Artificial Sequence
  CDRH3
DQLGEAFDI

SEQ ID NO: 138 - protein, 14 aa, Artificial Sequence
  CDRL1
TGSSSNIGAGYDVH

SEQ ID NO: 139 - protein, 7 aa, Artificial Sequence
  CDRL2
DNNKRPS

SEQ ID NO: 140 - protein, 11 aa, Artificial Sequence
  CDRL3
ATWDDSLSGPV

SEQ ID NO: 141 - protein, 5 aa, Artificial Sequence
  CDRH1
DYGMS

SEQ ID NO: 142 - protein, 17 aa, Artificial Sequence
  CDRH2
AISGSGSSTYYADSVKG

SEQ ID NO: 143 - protein, 8 aa, Artificial Sequence
  CDRH3
GDIDYFDY

SEQ ID NO: 144 - protein, 14 aa, Artificial Sequence
  CDRL1
TGSSSNFGAGYDVH

SEQ ID NO: 145 - protein, 7 aa, Artificial Sequence
  CDRL2
ENNKRPS

SEQ ID NO: 146 - protein, 11 aa, Artificial Sequence
  CDRL3
AAWDDSLNGPV

SEQ ID NO: 147 - protein, 5 aa, Artificial Sequence
  CDRH1
SYGMH

SEQ ID NO: 148 - protein, 17 aa, Artificial Sequence
  CDRH2
VISYDGSNKYYADSVKG

SEQ ID NO: 149 - protein, 8 aa, Artificial Sequence
  CDRH3
ERRDAFDI

SEQ ID NO: 150 - protein, 14 aa, Artificial Sequence
  CDRL1
TGSSSNIGAGYDVH

SEQ ID NO: 151 - protein, 7 aa, Artificial Sequence
  CDRL2
SDNQRPS

SEQ ID NO: 152 - protein, 9 aa, Artificial Sequence
  CDRL3
ATWDSDTPV

SEQ ID NO: 153 - protein, 5 aa, Artificial Sequence
  CDRH1
SYGMH

SEQ ID NO: 154 - protein, 17 aa, Artificial Sequence
  CDRH2
VISYDGSNKYYADSVKG

SEQ ID NO: 155 - protein, 10 aa, Artificial Sequence
  CDRH3
DHSAAGYFDY

SEQ ID NO: 156 - protein, 13 aa, Artificial Sequence
  CDRL1
SGSSSNIGSNTVN

SEQ ID NO: 157 - protein, 7 aa, Artificial Sequence
  CDRL2
GNSIRPS

SEQ ID NO: 158 - protein, 11 aa, Artificial Sequence
  CDRL3
ASWDDSLSSPV

SEQ ID NO: 159 - protein, 5 aa, Artificial Sequence
  CDRH1
SYGMH

SEQ ID NO: 160 - protein, 17 aa, Artificial Sequence
  CDRH2
GISWDSAIIDYAGSVKG

SEQ ID NO: 161 - protein, 10 aa, Artificial Sequence
  CDRH3
DEAAAGAFDI

SEQ ID NO: 162 - protein, 14 aa, Artificial Sequence
  CDRL1
TGSSSNIGAGYDVH

SEQ ID NO: 163 - protein, 7 aa, Artificial Sequence
  CDRL2
GNTDRPS

SEQ ID NO: 164 - protein, 12 aa, Artificial Sequence
  CDRL3
AAWDDSLSGPVV

SEQ ID NO: 165 - protein, 5 aa, Artificial Sequence
  CDRH1
SYGIS

SEQ ID NO: 166 - protein, 17 aa, Artificial Sequence
  CDRH2
GISGSGGNTYYADSVKG

SEQ ID NO: 167 - protein, 12 aa, Artificial Sequence
  CDRH3
SVGAYANDAFDI

SEQ ID NO: 168 - protein, 14 aa, Artificial Sequence
  CDRL1
TGSSSNIGAGYDVH

SEQ ID NO: 169 - protein, 7 aa, Artificial Sequence
  CDRL2
GDTNRPS

SEQ ID NO: 170 - protein, 11 aa, Artificial Sequence
  CDRL3
AAWDDSLNGPV

SEQ ID NO: 171 - protein, 5 aa, Artificial Sequence
  CDRH1
SYGMH

SEQ ID NO: 172 - protein, 17 aa, Artificial Sequence
  CDRH2
VISYDGSNKYYADSVKG

SEQ ID NO: 173 - protein, 8 aa, Artificial Sequence
  CDRH3
ELYDAFDI

SEQ ID NO: 174 - protein, 14 aa, Artificial Sequence
  CDRL1
TGSSSNIGAGYDVH

SEQ ID NO: 175 - protein, 7 aa, Artificial Sequence
  CDRL2
ADDHRPS

SEQ ID NO: 176 - protein, 11 aa, Artificial Sequence
  CDRL3
ASWDDSQRAVI

SEQ ID NO: 177 - protein, 5 aa, Artificial Sequence
  CDRH1
NYGMH

SEQ ID NO: 178 - protein, 17 aa, Artificial Sequence
  CDRH2
VISYDGSNKYYADSVKG

SEQ ID NO: 179 - protein, 8 aa, Artificial Sequence
  CDRH3
EYKDAFDI

SEQ ID NO: 180 - protein, 13 aa, Artificial Sequence
  CDRL1
TGSSSNIGSNTVN

SEQ ID NO: 181 - protein, 7 aa, Artificial Sequence
  CDRL2
DNNKRPS

SEQ ID NO: 182 - protein, 9 aa, Artificial Sequence
  CDRL3
QAWGTGIRV

SEQ ID NO: 183 - protein, 5 aa, Artificial Sequence
  CDRH1
SYGMH

SEQ ID NO: 184 - protein, 17 aa, Artificial Sequence
  CDRH2
VISYDGSNKYYADSVKG

SEQ ID NO: 185 - protein, 9 aa, Artificial Sequence
  CDRH3
EFGYIILDY

SEQ ID NO: 186 - protein, 13 aa, Artificial Sequence
  CDRL1
SGSSSNIGSNTVN

SEQ ID NO: 187 - protein, 7 aa, Artificial Sequence
  CDRL2
RDYERPS

SEQ ID NO: 188 - protein, 11 aa, Artificial Sequence
  CDRL3
MAWDDSLSGVV

SEQ ID NO: 189 - protein, 5 aa, Artificial Sequence
  CDRH1
NHGMH

SEQ ID NO: 190 - protein, 17 aa, Artificial Sequence
  CDRH2
VISYDGTNKYYADSVRG

SEQ ID NO: 191 - protein, 8 aa, Artificial Sequence
  CDRH3
ETWDAFDV

SEQ ID NO: 192 - protein, 13 aa, Artificial Sequence
  CDRL1
SGSSSNIGSNNAN

SEQ ID NO: 193 - protein, 7 aa, Artificial Sequence
  CDRL2
DNNKRPS

SEQ ID NO: 194 - protein, 9 aa, Artificial Sequence
  CDRL3
QAWDSSTVV

SEQ ID NO: 195 - protein, 447 aa, Artificial Sequence
  BI-1206 N297Q
EVQLLESGGGLVQPGGSLRLSCAASGFTFSSYGMHWVRQAPGKGLEWMAVISYDGSNKYYADSVKGRFTISRDNSKNTLYLQMNSLRAEDTAVYYCARELYDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 5 aa, Artificial Sequence
  CDRH1
SYGMH

SEQ ID NO: 197 - protein, 17 aa, Artificial Sequence
  CDRH2
VISYDGSNKYYADSVKG

SEQ ID NO: 198 - protein, 8 aa, Artificial Sequence
  CDRH3
ELYDAFDI

SEQ ID NO: 199 - protein, 14 aa, Artificial Sequence
  CDRL1
TGSSSNIGAGYDVH

SEQ ID NO: 200 - protein, 7 aa, Artificial Sequence
  CDRL2
ADDHRPS

SEQ ID NO: 201 - protein, 11 aa, Artificial Sequence
  CDRL3
ASWDDSQRAVI

SEQ ID NO: 202 - protein, 330 aa, Homo sapiens [sequence omitted]

SEQ ID NO: 203 - protein, 105 aa, Homo sapiens
  MISC_FEATURE (1)..(105): Lambda-CL
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0322933 A1
(43) Pub. Date: Oct. 12, 2023

(54) MODEL FOR PREDICTION OF TOLERABILITY ISSUES IN CONNECTION WITH INTRAVENOUS ADMINISTRATION OF THERAPEUTIC ANTIBODIES

(71) Applicant: BIOINVENT INTERNATIONAL AB, Lund (SE)

(72) Inventors: Björn FRENDÉUS, Lund (SE); Linda MARTENSSON, Bjärred (SE); Ingrid TEIGE, Lund (SE); Ingrid KARLSSON, Lund (SE)

(73) Assignee: BIOINVENT INTERNATIONAL AB, Lund (SE)

(21) Appl. No.: 18/008,134

(22) PCT Filed: Jun. 4, 2021
(86) PCT No.: PCT/EP2021/065013

§ 371 Date: Dec. 2, 2022

(30) Foreign Application Priority Data
Jun. 4, 2020 (EP) 20178287.7
Mar. 19, 2021 (EP) 21163703.8

(51) Int. Cl. C07K 16/28; A61K 49/00; A61K 31/573; A61K 31/341; A61K 9/00; A61K 39/395

(52) U.S. Cl. CPC C07K 16/283; A61K 49/0008; A61K 31/573; A61K 31/341; A61K 9/0019; A61K 39/3955; A61K 2039/54

(57) ABSTRACT

The present invention generally relates to combinations for use in therapeutic systems and antibody dosage regimens, and uses thereof. Also described herein is a model for predicting if a therapeutic antibody binding to a human target will be associated with a tolerability issue in connection with intravenous administration and/or for predicting if pre-treatment, altered administration route or modification of the antibody can prevent a tolerability issue associated with intravenous administration to a human of the therapeutic antibody. The model comprises administering the antibody intravenously or intraperitoneally to mice and observing the mice immediately after the administration for any transient display of the macroscopic symptoms isolation and decreased activity. The model may also comprise administration of a pre-treatment in combination with administration of the antibody, administration of the therapeutic antibody by a route of administration other than intravenous or intraperitoneal administration or administration of a modified format of the antibody to mice and observing the mice immediately after such administration for any transient display of the macroscopic symptoms isolation and decreased activity and comparing this with the transient display of the macroscopic symptoms isolation and decreased activity after the intravenous or intraperitoneal administration of the unmodified antibody without pre-treatment.

### CROSS-REFERENCE TO RELATED APPLICATIONS

This application is a national phase application under 35 U.S.C. § 371 of International Application No. PCT/EP2021/065013, filed Jun. 4, 2022, which claims the benefit of European Patent Application No. 21163703.8, filed Mar. 19, 2021, and European Patent Application No. 20178287.7, filed Jun. 4, 2020, the entirety of each of which are incorporated herein by reference.

### INCORPORATION OF SEQUENCE LISTING

The sequence listing that is contained in the file named “EPCLP0106US_Sequence_Listing_ST25.txt”, which is 94 kB (as measured in Microsoft Windows®) and was created on Jun. 22, 2023, is filed herewith by electronic submission and is incorporated by reference herein.

### FIELD OF THE INVENTION

The invention generally relates to therapeutic systems, combinations for use in dosage regimens, uses, methods, and kits for improving tolerability of an antibody molecule that binds specifically to FcγRIIb in a subject. The present invention also relates to a method or model that can be used to predict if a therapeutic antibody molecule binding specifically to a human target will be associated with a tolerability issue in connection with intravenous administration to a human, and/or to predict if a prophylactic or therapeutic treatment, an altered administration route and/or a modification of the therapeutic antibody molecule can prevent or mitigate a tolerability issue associated with intravenous administration to a human of a therapeutic antibody molecule binding specifically to a human target.

### BACKGROUND

Therapeutic antibodies constitute a well-proven class of drugs, which have been approved for therapy of diverse diseases including cancer, inflammatory diseases, autoimmune diseases, and infectious disease.

Monoclonal antibody therapies, in particular those used for cancer therapy, may be administered by intravenous infusion, allowing high immediate drug exposure that can be maintained through repeated dosing. In many cases however, the patient or subject may experience an adverse reaction to the infusion of the therapeutic antibody, which is termed an infusion-related reaction (“IRR”).

IRRs can be experienced by subjects during the infusion of the therapeutic antibody (a “uniphasic” reaction) and/or within hours of the infusion (a “biphasic” or “delayed” reaction), and they include hypersensitivity reactions and cytokine release syndromes (“CRS”). According to Common Terminology Criteria for Adverse Events (CTCAE) version 5.0 published Nov. 27, 2017 by the U.S. Department of Health and Human Services, the severity of adverse events, such as IRRs, is categorized in different grades, ranging from 1 (the least severe) to 5 (the most severe).

Common IRRs include but are not limited to respiratory conditions such as nasal congestion, cough, allergic rhinitis, throat irritation, and dyspnea, and non-respiratory conditions such as chills and nausea. Often the IRRs occur with the first dose administered to a subject, but they can also occur after the second or subsequent administration. In many cases the IRRs are mild, but more serious IRRs can sometimes occur which risk being fatal if not managed appropriately. An IRR may affect any organ system in the body.

Severe CRS can represent a life-threatening adverse event that requires prompt and aggressive treatment. Reduction of tumor burden, limitations on the dose of administered therapy, and premedication with steroids have reduced the incidence of severe CRS, as have the use of anti-cytokine treatments.

Tolerability issues may vary between different therapeutic antibodies and between subjects with varying frequency duration, severity and being of different nature.

The conventional management of hypersensitivity reactions, such as IRRs, includes temporary interruption of infusion, lowering the infusion rate, and/or treatment with antihistamines, antipyretics, and/or corticosteroids or in severe case interruption/cessation of infusions. In such severe cases, cautious re-introduction of infusions, at a slower rate with increases as tolerated may be considered. Pre-treatment with antipyretics and/or antihistamines may prevent reactions subsequent infusions.

Corticosteroids are often used to prevent or dampen infusion related reactions (IRR's) and associated toxicities seen with therapeutic antibodies. The corticosteroid regimen, i.e. type of corticosteroid, dose, and timing of administration, depends both on the therapeutic antibody of use and on the indication. Rituxan (rituximab) is a CD20-directed cytolytic antibody commonly used both in CD20-positive B cell lymphomas (Non-Hodgkin's Lymphoma (NHL) and Chronic Lymphocytic Leukemia (CLL)) as well as chronic inflammatory disorders such as Rheumatoid Arthritis (RA). In the case of NHL and CLL, corticosteroids are often used to lower the risk of IRR's and then administrated 30 minutes prior to the first rituximab cycle, and only at subsequent cycles if severe infusion-related adverse events were experienced during the first cycle. In the case of NHL corticosteroids (i.e. prednisone) are also used as part of in combination therapy. i.e. rituximab, cyclophosphamide, doxorubicin, vincristine, and prednisone (R-CHOP). In the case of RA, corticosteroid is recommended 30 minutes prior to each infusion. When administering another CD20-directed antibody Gazyva (obinutuzumab), corticosteroid premedication is also recommended prior to the first treatment cycle and prior to following cycles only in patients where grade 3 IRR was experienced with the previous infusion or with a lymphocyte count >25×10⁹/L prior to next treatment. In the case of Gazyva corticosteroid premedication should be given at least 1 hour prior to the antibody infusion. A third example of a therapeutic antibody where corticosteroids are used to lower the risk of IRR's is Darzalex (daratumumab), a CD38-directed antibody indicated for the treatment of patients with multiple myeloma. Corticosteroids are in this case recommended pre and post every infusion, 1-3 hour prior to infusion and then again on each of the 2 days following infusion.

WO 2020/047389 describes dosing strategies and administration regimens for therapeutic protein, such as antibodies (e.g., bispecific antibodies targeting T cells), that mitigate the prevalence and severity of cytokine release syndrome or an infusion-related reaction in patients undergoing immunotherapy. comprising: (i) administering fractions of a primary dose (D1) of the therapeutic protein in week 1 of the dosing regimen, wherein the primary dose comprises no more than 10 mg of the therapeutic protein, a first dose fraction (F1D1) comprises 40% to 60% of the total primary dose and is administered to the subject on day 1 of week 1, and a second dose fraction (F2D1) comprises the remaining 40% to 60% of the total primary dose and is administered to the subject from 12 to 96 hours following administration of the F1D1; (ii) administering fractions of a secondary dose (D2) of the therapeutic protein in week 2 of the dosing regimen, wherein the secondary dose is no more than one-half of a maximum weekly dose of the therapeutic protein, a first dose fraction (F1D2) comprises 40% to 60% of the total secondary dose, a second dose fraction (F2D2) comprises the remaining 40% to 60% of the total secondary dose, and the F2D2 is administered to the subject from 12 to 96 hours following administration of the F1D2 during week 2 of the dosing regimen; and (iii) administering the maximum weekly dose of the therapeutic protein to the subject as a single dose in a subsequent week of the dosing regimen. Fractionated dosing is not ideal, since dosing with suboptimally efficacious doses risk limiting therapeutic benefit, in the worst case resulting in no clinical benefit of the intended therapeutic treatment or induction of disease progression.

WO 2020/037024 mentions the use of an additional therapeutic agent, such as an antihistamine, acetaminophen or a corticosteroid, to prevent or reduce the severity of adverse events, such as an infusion-related reaction, in treatment of ovarian cancer, peritoneal cancer or fallopian tube cancer with an anti-tissue factor antibody or an antigen-binding fragment thereof conjugated to a monomethyl auristatin or a functional analog or derivative thereof or a functional derivative thereof.

Management of the toxicities associated with immunotherapy is a challenging clinical problem. Methods to reduce, suppress or overcome the tolerability issues associated with intravenous administration of different antibodies are greatly needed. However, the heterogeneity in nature and frequency of tolerability issues associated with antibodies to different targets, and the poor molecular and cellular understanding of the mechanisms underlying these, mean that numerous disparate approaches have been developed, and the effectiveness of each can vary significantly depending on the type of therapeutic antibody with which they are used.

The above demonstrates that intravenous administration of antibodies to different targets often is associated with tolerability issues. Such tolerability issues may vary between different therapeutic antibodies and between patients with varying frequency duration, severity and being of different nature.

Accordingly, methods to reduce, suppress or overcome the different tolerability issues associated with iv administration of different antibodies directed to the same target (e.g. anti-CD20 antibodies rituximab compared with Obinutuzumab) or to different targets (e.g. anti-CD38 antibodies compared with anti-CD20 antibodies) differ greatly, and comprise administration of different agents e.g. corticosteroids or antihistamines immediately before, concomitantly, and/or following intravenous administration of the therapeutic antibody.

There is a great need and value of methods enabling prediction of whether or not tolerability issues are likely to occur in association with intravenous administration of antibodies to different targets, and equally importantly of methods enabling discovery of means that help prevent, suppress or overcome tolerability issues associated with iv administration of antibodies to given targets. Preclinical methods allowing for such prediction and screening at early stages of therapeutic antibody development at a relatively lower cost and higher throughput compared to the human clinical setting are of outstanding importance.

### SUMMARY AND DETAILED DESCRIPTION

Against this background, the inventors have developed a surprisingly advantageous approach for administering an antibody molecule that specifically binds to FcγRIIb in a subject. As demonstrated in the accompanying Examples, the inventors' approach maintains the therapeutic effectiveness of such an antibody, whilst reducing and/or preventing IRRs associated with its administration. The inventors' approach involves administering several separate doses of the antibody, including an initial sub-maximal therapeutic dose of the antibody, and performing that antibody administration after a corticosteroid has been given to the subject. The inventors' approach therefore provides an improved regimen for administering such antibodies, as it does so in a way that reduces and/or prevents tolerability issues in the subject.

In addition, the inventors have developed a method or model that can be used to predict if a therapeutic antibody molecule binding specifically to a human target will be associated with a tolerability issue in connection with intravenous administration to a human, and/or to predict if a prophylactic or therapeutic treatment, an altered administration route and/or a modification of the therapeutic antibody molecule can prevent or mitigate a tolerability issue associated with intravenous administration to a human of a therapeutic antibody molecule binding specifically to a human target.

### First to Fifth Aspects of the Invention

In a first aspect, the invention provides a therapeutic system for use in improving tolerability of an antibody molecule that specifically binds to FcγRIIb in a subject, wherein the therapeutic system comprises:
(i) an antibody molecule that specifically binds to FcγRIIb, wherein the antibody molecule is administered to the subject as at least a first dose and a second dose; and
(ii) a corticosteroid,
   wherein the first dose of the antibody molecule is lower than the maximum therapeutically effective dose of the antibody molecule; and wherein the corticosteroid is administered to the subject before the first dose of the antibody molecule.

In a second aspect, the invention provides a combination comprising an antibody molecule and a corticosteroid for use in a dosage regimen for improving tolerability of an antibody molecule that specifically binds to FcγRIIb in a subject, wherein the dosage regimen comprises the following steps:
(i) administration of a corticosteroid before administration of a first dose of the antibody molecule;
(ii) administration of the first dose of the antibody molecule that specifically binds to FcγRIIb that is lower than the maximum therapeutically effective dose; and
(iii) administration of a second dose (and, preferably, at least a second dose) of the antibody molecule that specifically binds to FcγRIIb, wherein the first dose of the antibody molecule is administered prior to the second dose.

In a third aspect, the invention provides use of:
(i) an antibody molecule that specifically binds to FcγRIIb; and
(ii) a corticosteroid,
   in the manufacture of a medicament for improving tolerability of an antibody molecule that specifically binds to FcγRIIb in a subject, wherein the medicament comprises at least a first dose and a second dose of the antibody molecule; and wherein the first dose of the antibody molecule is lower than the maximum therapeutically effective dose of the antibody molecule; and wherein the corticosteroid is administered before the first dose of the antibody molecule.

In a fourth aspect, the invention provides a method for improving tolerability of an antibody molecule that specifically binds to FcγRIIb in a subject comprising:
(i) administration of a corticosteroid before administration of a first dose of the antibody molecule;
(ii) administration of the first dose of the antibody molecule that specifically binds to FcγRIIb that is lower than the maximum therapeutically effective dose; and
(iii) administration of a second dose (and, preferably, at least a second dose) of the antibody molecule that specifically binds to FcγRIIb, wherein the first dose of the antibody molecule is administered prior to the second dose.

The inventors have surprisingly found that a combination of a dose of a corticosteroid, followed by a first dose of the antibody molecule that specifically binds to FcγRIIb that is lower than the maximum therapeutically effective dose, followed by a second dose of the antibody molecule leads to surprising improvements in the tolerability of the antibody molecule that specifically binds FcγRIIb.

Antibody molecules are well known to those skilled in the art of immunology and molecular biology. Typically, an antibody comprises two heavy (H) chains and two light (L) chains. Herein, we sometimes refer to this complete antibody molecule as a full-size or full-length antibody. The antibody's heavy chain comprises one variable domain (VH) and three constant domains (CH1, CH2 and CH3), and the antibody's molecule light chain comprises one variable domain (VL) and one constant domain (CL). The variable domains (sometimes collectively referred to as the Fv region) bind to the antibody's target, or antigen. Each variable domain comprises three loops, referred to as complementary determining regions (CDRs), which are responsible for target binding. The constant domains are not involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and in humans several of these are further divided into subclasses (isotypes), e.g., IgG1, IgG2, IgG3, and IgG4; IgA1 and IgA2.

Another part of an antibody is the Fc region (otherwise known as the fragment crystallisable domain), which comprises two of the constant domains of each of the antibody's heavy chains. As mentioned herein, the Fc region is responsible for interactions between the antibody and Fc receptor.

The term antibody molecule, as used herein, encompasses full-length or full-size antibodies as well as functional fragments of full length antibodies and derivatives of such antibody molecules.

Functional fragments of a full-size antibody have the same antigen binding characteristics as the corresponding full-size antibody and include either the same variable domains (i.e. the VH and VL sequences) and/or the same CDR sequences as the corresponding full-size antibody. That the functional fragment has the same antigen binding characteristics as the corresponding full-size antibody means that it binds to the same epitope on the target as the full-size antibody. Such a functional fragment may correspond to the Fv part of a full-size antibody. Alternatively, such a fragment may be a Fab, also denoted F(ab), which is a monovalent antigen-binding fragment that does not contain a Fc part, or a F(ab′)₂, which is an divalent antigen-binding fragment that contains two antigen-binding Fab parts linked together by disulfide bonds, or a F(ab′), i.e. a monovalent-variant of a F(ab′)₂. Such a fragment may also be single chain variable fragment (scFv).

A functional fragment does not always contain all six CDRs of a corresponding full-size antibody. It will be appreciated that molecules containing three or fewer CDR regions (in some cases, even just a single CDR or a part thereof) are capable of retaining the antigen-binding activity of the antibody from which the CDR(s) are derived. For example, in Gao et al., 1994, J. Biol. Chem., 269: 32389-93 it is described that a whole VL chain (including all three CDRs) has a high affinity for its substrate.

Molecules containing two CDR regions are described, for example, by Vaughan & Sollazzo 2001, Combinatorial Chemistry & High Throughput Screening, 4: 417-430. On page 418 (right column—3 Our Strategy for Design) a minibody including only the H1 and H2 CDR hypervariable regions interspersed within framework regions is described. The minibody is described as being capable of binding to a target. Pessi et al., 1993, Nature, 362: 367-9 and Bianchi et al., 1994, J. Mol. Biol., 236: 649-59 are referenced by Vaughan &Sollazzo and describe the H1 and H2 minibody and its properties in more detail. In Qiu et al., 2007, Nature Biotechnology, 25:921-9 it is demonstrated that a molecule consisting of two linked CDRs are capable of binding antigen. Quiocho 1993, Nature, 362: 293-4 provides a summary of “minibody” technology. Ladner 2007, Nature Biotechnology, 25:875-7 comments that molecules containing two CDRs are capable of retaining antigen-binding activity.

Antibody molecules containing a single CDR region are described, for example, in Laune et al., 1997, JBC, 272: 30937-44, in which it is demonstrated that a range of hexapeptides derived from a CDR display antigen-binding activity and it is noted that synthetic peptides of a complete, single, CDR display strong binding activity. In Monnet et al., 1999, JBC, 274: 3789-96 it is shown that a range of 12-mer peptides and associated framework regions have antigen-binding activity and it is commented on that a CDR3-like peptide alone is capable of binding antigen. In Heap et al., 2005, 3. Gen. Virol., 86: 1791-1800 it is reported that a “micro-antibody” (a molecule containing a single CDR) is capable of binding antigen and it is shown that a cyclic peptide from an anti-HIV antibody has antigen-binding activity and function. In Nicaise et al., 2004, Protein Science, 13:1882-91 it is shown that a single CDR can confer antigen-binding activity and affinity for its lysozyme antigen.

Thus, antibody molecules having five, four, three or fewer CDRs are capable of retaining the antigen binding properties of the full-length antibodies from which they are derived.

The antibody molecule may also be a derivative of a full-length antibody or a fragment of such an antibody. When a derivative is used it should have the same antigen binding characteristics as the corresponding full-length antibody in the sense that it binds to the same epitope on the target as the full-length antibody.

Thus, by the term “antibody molecule”, as used herein, we include all types of antibody molecules and functional fragments thereof and derivatives thereof, including: monoclonal antibodies, polyclonal antibodies, synthetic antibodies, recombinantly produced antibodies, multi-specific antibodies, bi-specific antibodies, human antibodies, antibodies of human origin, humanized antibodies, chimeric antibodies, single chain antibodies, single-chain Fvs (scFv), Fab fragments, F(ab′)₂ fragments, F(ab′) fragments, disulfide-linked Fvs (sdFv), antibody heavy chains, antibody light chains, homo-dimers of antibody heavy chains, homo-dimers of antibody light chains, heterodimers of antibody heavy chains, heterodimers of antibody light chains, antigen binding functional fragments of such homo- and heterodimers.

Further, the term “antibody molecule”, as used herein, includes all classes of antibody molecules and functional fragments, including: IgG, IgG1, IgG2, IgG3, IgG4, IgA, IgM, IgD, and IgE, unless otherwise specified.

As outlined above, different types and forms of antibody molecules are encompassed by the invention, and would be known to the person skilled in immunology. It is well known that antibodies used for therapeutic purposes are often modified with additional components which modify the properties of the antibody molecule.

Accordingly, we include that an antibody molecule of the invention or an antibody molecule used in accordance with the invention (for example, a monoclonal antibody molecule, and/or polyclonal antibody molecule, and/or bi-specific antibody molecule) comprises a detectable moiety and/or a cytotoxic moiety.

By “detectable moiety”, we include one or more from the group comprising of: an enzyme; a radioactive atom; a fluorescent moiety; a chemiluminescent moiety; a bioluminescent moiety. The detectable moiety allows the antibody molecule to be visualised in vitro, and/or in vivo, and/or ex vivo.

By “cytotoxic moiety”, we include a radioactive moiety, and/or enzyme, wherein the enzyme is a caspase, and/or toxin, wherein the toxin is a bacterial toxin or a venom; wherein the cytotoxic moiety is capable of inducing cell lysis.

We further include that the antibody molecule may be in an isolated form and/or purified form, and/or may be PEGylated. PEGylation is a method by which polyethylene glycol polymers are added to a molecule such as an antibody molecule or derivative to modify its behaviour, for example to extend its half-life by increasing its hydrodynamic size, preventing renal clearance.

As discussed above, the CDRs of an antibody bind to the antibody target. The assignment of amino acids to each CDR described herein is in accordance with the definitions according to Kabat E A et al. 1991, In “Sequences of Proteins of Immunological Interest” Fifth Edition, NIH Publication No. 91-3242, pp xv-xvii.

As the skilled person would be aware, other methods also exist for assigning amino acids to each CDR. For example, the International ImMunoGeneTics information system (IMGT®) (http://www.imgt.org/and Lefranc and Lefranc “The Immunoglobulin FactsBook” published by Academic Press, 2001).

In some embodiments, the antibody molecule specifically binds to FcγRIIb. Fc receptors are well known in the art as membrane proteins which are found on the cell surface of immune effector cells, such as macrophages. The name is derived from their binding specificity for the Fc region of antibodies, which is the usual way an antibody binds to the receptor. However, certain antibodies can also bind the Fc receptors via the antibodies' complementarity determining regions (“CDR”) sequences in the case of antibodies specifically binding to one or more Fc receptors.

A subgroup of the Fc receptors are Fcγ receptors (Fc-gamma receptors, FcgammaR), which are specific for IgG antibodies. There are two types of Fcγ receptors: activating Fcγ receptors (also denoted activatory Fcγ receptors) and inhibitory Fcγ receptors. The activating and the inhibitory receptors transmit their signals via immunoreceptor tyrosine-based activation motifs (ITAM) or immunoreceptor tyrosine-based inhibitory motifs (ITIM), respectively. In humans, FcγRIIb (CD32b) is an inhibitory Fcγ receptor, while FcγRI (CD64), FcγRIIa (CD32a), FcγRIIc (CD32c), FcγRIIIa (CD16a) and FcγRIV are activating Fcγ receptors. FcγRIIIb is a GPI-linked receptor expressed on neutrophils that lacks an ITAM motif but through its ability to cross-link lipid rafts and engage with other receptors is also considered activatory. In mice, the activating receptors are FcγRI, FcγRIII and FcγRIV.

It is well-known that antibodies modulate immune cell activity through interaction with Fcγ receptors. Specifically, how antibody immune complexes modulate immune cell activation is determined by their relative engagement of activating and inhibitory Fcγ receptors. Different antibody isotypes bind with different affinity to activating and inhibitory Fcγ receptors, resulting in different A:I ratios (activation:inhibition ratios) (Nimmerjahn et al; Science. 2005 Dec. 2; 310(5753):1510-2).

By binding to an inhibitory Fcγ receptor, an antibody can inhibit, block and/or downmodulate effector cell functions.

By binding to an activatory Fcγ receptor, an antibody can activate effector cell functions and thereby trigger mechanisms such as antibody-dependent cellular cytotoxicity (ADCC), antibody dependent cellular phagocytosis (ADCP), cytokine release, and/or antibody dependent endocytosis, as well as NETosis (i.e. activation and release of NETs, Neutrophil extracellular traps) in the case of neutrophils. Antibody binding to an activating Fcγ receptor can also lead to an increase in certain activation markers, such as CD40, MHCII, CD38, CD80 and/or CD86.

The antibody molecule according to the invention that specifically binds FcγRIIb, binds to or interacts with this Fcγ receptor via the Fab region of the antibody, i.e. via the antigen-binding region on an antibody that binds to antigens which is composed of one constant and one variable domain of each of the heavy and the light chain. In particular, it binds to FcγRIIb present on an immune effector cell, and in particular to FcγRIIb present on the surface of an immune effector cell.

In some preferred embodiments, the antibody molecule according to the invention that specifically binds FcγRIIb can also bind to Fcγ receptors via its Fc region. In some embodiments, these are activatory or inhibitory Fcγ receptors. In some preferred embodiments, the antibody molecule may be an IgG1, IgG2, IgG3, or IgG4 type antibody molecule.

In some other embodiments, the antibody molecule that specifically binds FcγRIIb may be engineered for enhanced binding to Fcγ receptors via its Fc region, for example via afucosylation.

In some other embodiments, the antibody molecule according to the invention has reduced or impaired binding for Fcγ receptors via its Fc region. It is well known that aglycosylation of antibodies, specifically in the 297 position (such as one of the following mutations: N297A, N297Q or N297G), render both human and mouse IgG impaired for binding to FcγR. The antibody molecule may also have reduced or impaired binding if it lacks an Fc region. Furthermore, impaired or abrogated FcγR binding means that the modified format does not bind at all to FcγR or that it binds less strongly to FcγR than the unmodified antibody.

By “reduced binding to Fcγ receptors” (also referred to as “binding with reduced affinity”) we include that the antibody molecule has reduced Fc mediated binding to Fcγ receptors, or in other words that the Fc region of the antibody molecule that specifically binds FcγRIIb binds to an activating Fcγ receptor with lower affinity than the Fc region of a normal human IgG1. The reduction in binding can be assessed using techniques such as surface plasmon resonance. In this context “normal IgG1” means a conventionally produced IgG1 with a non-mutated Fc region that has not been produced so as to alter its glycosylation. As a reference for this “normal IgG1” it is possible to use rituximab produced in CHO cells without any modifications (Tipton et al, Blood 2015 125:1901-1909; rituximab is described in, for example, EP 0 605 442). Human IgG2 and human IgG4 are examples of antibody isotypes that bind with reduced affinity to Fcγ receptors compared with human IgG1. Therefore, antibodies based on human IgG2 and IgG4 have “reduced binding to Fcγ receptors” within the meaning of this term.

In some other embodiments, the antibody molecule according to the invention may not have an Fc region (and therefore cannot bind Fcγ receptors via an Fc region). Such fragments are discussed above and include Fv, Fab (also denoted F(ab)), F(ab′)₂, F(ab′), or scFv. The antibody molecule according to the invention may also be a bi-specific antibody fragment, for example an scFv, Fab, or Fab′2, specific for FcgRIIB and an additional FcgR.

The therapeutic antibody molecule may then be an antibody molecule described in WO 2012/022985, WO 2015/173384 and/or WO 2019/138005. In some embodiments it is an antibody having the CDR sequences SEQ ID Nos: 83-88 as described in WO 2012/022985. In some embodiments, it is an antibody having a VH with Seq ID No: 12 and a VL with Seq ID No: 25 as described in WO 2012/022985.

In some embodiments, it is the antibody described in WO 2012/022985 as having a VH with Seq ID No: 12, a VL with Seq ID No: 25; a CH with Seq ID No: 1 and a CL with Seq ID No: 2 (corresponding to the antibody disclosed herein with a light chain having SEQ. ID. No: 1 and a heavy chain having SEQ. ID. No: 2). In some preferred embodiments, the antibody molecule of the invention has a light chain with SEQ ID No:1. In some additional embodiments, the antibody molecule of the invention has a heavy chain with SEQ ID No:2.

Light chain:
(SEQ ID No: 1)
QSVLTQPPSASGTPGQRVTISCTGSSSNIGAGYDVHWYQQLPGTAPKLLI
YADDHRPSGVPDRFSGSKSGTSASLAISGLRSEDEADYYCASWDDSQRAV
IFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS
Heavy chain:
(SEQ ID NO: 2)
EVQLLESGGGLVQPGGSLRLSCAASGFTFSSYGMHWVRQAPGKGLEWMAV
ISYDGSNKYYADSVKGRFTISRDNSKNTLYLQMNSLRAEDTAVYYCAREL
YDAFDIWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

In some embodiments, the antibody molecule of the invention has a light chain with SEQ ID No:1 and a heavy chain with SEQ ID No:2 (this antibody is denoted as BI-1206).

As mentioned above, the antibody molecule of the invention, may in some embodiments, have reduced or impaired binding for Fcγ receptors via its Fc region. In this case, the therapeutic antibody molecule is an Fc receptor binding antibody and the modified format is an antibody having the same Fv variable sequence but having impaired or abrogated FcγR binding compared with the therapeutic antibody molecule.

In some embodiments, the therapeutic antibody is an Fc receptor binding anti-FcγRIIB antibody, and in some such cases, the modified format is anti-FcγRIIB antibody is the antibody having a light chain with SEQ ID No:1 and a heavy chain with SEQ ID No: 195.

A modified format of BI-1206 is format wherein the glycosylation site at N297 (marked in bold above in SEQ ID NO:2) is mutated to a Q (marked in bold below), i.e. an N297Q mutation, resulting in the following heavy chain:

(SEQ. ID. No: 195)
EVQLLESGGGLVQPGGSLRLSCAASGFTFSSYGMHWVRQAPGKGLEWMAV
ISYDGSNKYYADSVKGRFTISRDNSKNTLYLQMNSLRAEDTAVYYCAREL
YDAFDIWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYQSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

The CDR regions of the light chain of SEQ ID No:1, and the CDR regions of the heavy chain of SEQ ID No:2 or 195, are shown below:

Heavy chain CDRs:
CDRH1:
(SEQ ID No: 196)
SYGMH
CDRH2:
(SEQ ID No: 197)
VISYDGSNKYYADSVKG
CDRH3:
(SEQ ID No: 198)
ELYDAFDI
Light chain CDRs:
CDRL1:
(SEQ ID No: 199)
TGSSSNIGAGYDVH
CDRL2:
(SEQ ID No: 200)
ADDHRPS
CDRL3:
(SEQ ID No: 201)
ASWDDSQRAVI

Accordingly, in some embodiments, the antibody molecule of the invention comprises one or more of the CDR sequences of SEQ ID No:196-201. For example, the antibody molecule comprises two or more, or three or more, or four or more, or five or more, or all six of the CDR sequences of SEQ ID No:196-201. For example, the antibody molecule may comprise: one or more, or two or more, or three of the light chain CDR regions (i.e. SEQ ID No:199, 200, and 201); and/or one or more, or two or more, or three of the heavy chain CDR regions (i.e. SEQ ID No:196, 197, and 198).

Preferably, the antibody molecule of the invention comprises the following constant regions (CH and CL):

IgG1-CH [SEQ ID No: 202]:
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
λ-CL [SEQ ID No: 203]:
QPKAAPSVTLFPPSSEELQANKATLVCLISDFYPGAVTVAWKADSSPVKA
GVETTTPSKQSNNKYAASSYLSLTPEQWKSHRSYSCQVTHEGSTVEKTVA
PTECS

Thus, in a preferred embodiment, the antibody molecule of the invention comprises:
A light chain with SEQ ID No:1, and a heavy chain with SEQ ID No:2, and constant regions with SEQ ID No:202 and 203; or
A light chain with SEQ ID No:1, and a heavy chain with SEQ ID No:195, and constant regions with SEQ ID No:202 and 203.

In alternative embodiments, the antibody molecule that specifically binds FcγRIIb is an antibody as described in published PCT patent applications WO 2012/022985, WO 2015/173384 and/or WO 2019/138005.

The antibody that specifically binds FcγRIIb may comprise one or more sequences of the following clones:

Antibody clone: 1A01
1A01-VH
[SEQ ID NO: 3]
EVQLLESGGGLVQPGGSLRLSCAASGFTFSDYYMNWIRQTPGKGLEWVSLIGWDGGSTYYADSVK
GRFTISRDNSENTLYLQMNSLRAEDTAVYYCARAYSGYELDYWGQGTLVTVSS
1A01-VL
[SEQ ID NO: 27]
QSVLTQPPSASGTPGQRVTISCSGSSSNIGNNAVNWYQQLPGTAPKLLIYDNNNRPSGVPDRFSGS
KSGTSASLAISGLRSEDEADYYCAAWDDSLNASIFGGGTKLTVLG
CDR regions
CDRH1:
[SEQ ID NO: 51]
DYYMN
CDRH2:
[SEQ ID NO: 52]
LIGWDGGSTYYADSVKG
CDRH3:
[SEQ ID NO: 53]
AYSGYELDY
CDRL1:
[SEQ ID NO: 54]
SGSSSNIGNNAVN
CDRL2:
[SEQ ID NO: 55]
DNNNRPS
CDRL3:
[SEQ ID NO: 56]
AAWDDSLNASI
Antibody clone: 1B07
1B07-VH
[SEQ ID NO: 4]
EVQLLESGGGLVQPGGSLRLSCAASGFTFSSYGMHWVRQAPGKGLEWVAFTRYDGSNKYYADSVR
GRFTISRDNSKNTLYLQMNSLRAEDTAVYYCARENIDAFDVWGQGTLVTVSS
1B07-VL
[SEQ ID NO: 28]
QSVLTQPPSASGTPGQRVTISCSGSSSNIGNNAVNWYQQLPGTAPKLLIYDNQQRPSGVPDRFSGS
KSGTSASLAISGLRSEDEADYYCEAWDDRLFGPVFGGGTKLTVLG
CDR regions
CDRH1:
[SEQ ID NO: 57]
SYGMH
CDRH2:
[SEQ ID NO: 58]
FTRYDGSNKYYADSVRG
CDRH3:
[SEQ ID NO: 59]
ENIDAFDV
CDRL1:
[SEQ ID NO: 60]
SGSSSNIGNNAVN
CDRL2:
[SEQ ID NO: 61]
DNQQRPS
CDRL3:
[SEQ ID NO: 62]
WDDRLFGPV
Antibody clone: 1C04
1C04-VH
[SEQ ID NO: 5]
EVQLLESGGGLVQPGGSLRLSCAASGFTFSSYAMSWVRQAPGKGLEWVSSISDSGAGRYYADSVE
GRFTISRDNSKNTLYLQMNSLRAEDTAVYYCARTHDSGELLDAFDIWGQGTLVTVSS
1C04-VL
[SEQ ID NO: 29]
QSVLTQPPSASGTPGQRVTISCSGSSSNIGSNHVLWYQQLPGTAPKLLIYGNSNRPSGVPDRFSGS
KSGTSASLAISGLRSEDEADYYCAAWDDSLNGWVFGGGTKLTVLG
CDR regions
CDRH1:
[SEQ ID NO: 63]
SYAMS
CDRH2:
[SEQ ID NO: 64]
SISDSGAGRYYADSVEG
CDRH3:
[SEQ ID NO: 65]
THDSGELLDAFDI
CDRL1:
[SEQ ID NO: 66]
SGSSSNIGSNHVL
CDRL2:
[SEQ ID NO: 67]
GNSNRPS
CDRL3:
[SEQ ID NO: 68]
AAWDDSLNGWV
Antibody clone: 1E05
1E05-VH
[SEQ ID NO: 6]
EVQLLESGGGLVQPGGSLRLSCAASGFTFSTYAMNWVRQVPGKGLEWVAVISYDGSNKNYVDSVK
GRFTISRDNSKNTLYLQMNSLRAEDTAVYYCARNFDNSGYAIPDAFDIWGQGTLVTVSS
1E05-VL
[SEQ ID NO: 30]
QSVLTQPPSASGTPGQRVTISCTGSSSNIGAGYDVHWYQQLPGTAPKLLIYDNNSRPSGVPDRFSG
SKSGTSASLAISGLRSEDEADYYCAAWDDSLGGPVFGGGTKLTVLG
CDR regions
CDRH1:
[SEQ ID NO: 69]
TYAMN
CDRH2:
[SEQ ID NO: 70]
VISYDGSNKNYVDSVKG
CDRH3:
[SEQ ID NO: 71]
NFDNSGYAIPDAFDI
CDRL1:
[SEQ ID NO: 72]
TGSSSNIGAGYDVH
CDRL2:
[SEQ ID NO: 73]
DNNSRPS
CDRL3:
[SEQ ID NO: 74]
AAWDDSLGGPV
Antibody clone: 2A09
2A09-VH
[SEQ ID NO: 7]
EVQLLESGGGLVQPGGSLRLSCAASGFTFSNAWMSWVRQAPGKGLEWVAYISRDADITHYPASVK
GRFTISRDNSKNTLYLQMNSLRAEDTAVYYCTTGFDYAGDDAFDIWGQGTLVTVSS
2A09-VL
[SEQ ID NO: 31]
QSVLTQPPSASGTPGQRVTISCSGSSSNIGSNAVNWYQQLPGTAPKLLIYGNSDRPSGVPDRFSGS
KSGTSASLAISGLRSEDEADYYCAAWDDSLNGRWVFGGGTKLTVLG
CDR regions
CDRH1:
[SEQ ID NO: 75]
NAWMS
CDRH2:
[SEQ ID NO: 76]
YISRDADITHYPASVKG
CDRH3:
[SEQ ID NO: 77]
GFDYAGDDAFDI
CDRL1:
[SEQ ID NO: 78]
SGSSSNIGSNAVN
CDRL2:
[SEQ ID NO: 79]
GNSDRPS
CDRL3:
[SEQ ID NO: 80]
AAWDDSLNGRWV
Antibody clone: 2B08
2B08-VH
[SEQ ID NO: 8]
EVQLLESGGGLVQPGGSLRLSCAASGFTFSDYYMSWVRQAPGKGLEWVALIGHDGNNKYYLDSLE
GRFTISRDNSKNTLYLQMNSLRAEDTAVYYCARATDSGYDLLYWGQGTLVTVSS
2B08-VL
[SEQ ID NO: 32]
QSVLTQPPSASGTPGQRVTISCSGSSSNIGNNAVNWYQQLPGTAPKLLIYYDDLLPSGVPDRFSGS
KSGTSASLAISGLRSEDEADYYCTTWDDSLSGVVFGGGTKLTVLG
CDR regions
CDRH1:
[SEQ ID NO: 81]
DYYMS
CDRH2:
[SEQ ID NO: 82]
LIGHDGNNKYYLDSLEG
CDRH3:
[SEQ ID NO: 83]
ATDSGYDLLY
CDRL1:
[SEQ ID NO: 84]
SGSSSNIGNNAVN
CDRL2:
[SEQ ID NO: 85]
YDDLLPS
CDRL3:
[SEQ ID NO: 86]
TTWDDSLSGVV
Antibody clone: 2E08
2E08-VH
[SEQ ID NO: 9]
EVQLLESGGGLVQPGGSLRLSCAASGFTFSDYYMSWIRQAPGKGLEWVSAIGFSDDNTYYADSVK
GRFTISRDNSKNTLYLQMNSLRAEDTAVYYCAGGDGSGWSFWGQGTLVTVSS
2E08-VL
[SEQ ID NO: 33]
QSVLTQPPSASGTPGQRVTISCSGSSSNIGNNAVNWYQQLPGTAPKLLIYDNNKRPSGVPDRFSGS
KSGTSASLAISGLRSEDEADYYCATWDDSLRGWVFGGGTKLTVLG
CDR regions
CDRH1:
[SEQ ID NO: 87]
DYYMS
CDRH2:
[SEQ ID NO: 88]
AIGFSDDNTYYADSVKG
CDRH3:
[SEQ ID NO: 89]
GDGSGWSF
CDRL1:
[SEQ ID NO: 90]
SGSSSNIGNNAVN
CDRL2:
[SEQ ID NO: 91]
DNNKRPS
CDRL3:
[SEQ ID NO: 92]
ATWDDSLRGWV
Antibody clone: 5C04
5C04-VH
[SEQ ID NO: 10]
EVQLLESGGGLVQPGGSLRLSCAASGFTFSNYGMHWVRQAPGKGLEWVAVISYDGSNKYYADSVK
GRFTISRDNSKNTLYLQMNSLRAEDTAVYYCAREWRDAFDIWGQGTLVTVSS
5C04-VL
[SEQ ID NO: 34]
QSVLTQPPSASGTPGQRVTISCTGSSSNIGAGYDVHWYQQLPGTAPKLLIYSDNQRPSGVPDRFSG
SKSGTSASLAISGLRSEDEADYYCAAWDDSLSGSWVFGGGTKLTVLG
CDR regions
CDRH1:
[SEQ ID NO: 93]
NYGMH
CDRH2:
[SEQ ID NO: 94]
VISYDGSNKYYADSVKG
CDRH3:
[SEQ ID NO: 95]
WRDAFDI
CDRL1:
[SEQ ID NO: 96]
TGSSSNIGAGYDVH
CDRL2:
[SEQ ID NO: 97]
SDNQRPS
CDRL3:
[SEQ ID NO: 98]
AAWDDSLSGSWV
Antibody clone: 5C05
5C05-VH
[SEQ ID NO: 11]
EVQLLESGGGLVQPGGSLRLSCAASGFTFSTYGMHWVRQAPGKGLEWVAVISYDGSNKYYADSVK
GRFTISRDNSKNTLYLQMNSLRAEDTAVYYCARENFDAFDVWGQGTLVTVSS
5C05-VL
[SEQ ID NO: 35]
QSVLTQPPSASGTPGQRVTISCTGSSSNIGAGYDVHWYQQLPGTAPKLLIYSNSQRPSGVPDRFSG
SKSGTSASLAISGLRSEDEADYYCAAWDDSLNGQVVFGGGTKLTVLG
CDR regions
CDRH1:
[SEQ ID NO: 99]
TYGMH
CDRH2:
[SEQ ID NO: 100]
VISYDGSNKYYADSVKG
CDRH3:
[SEQ ID NO: 101]
ENFDAFDV
CDRL1:
[SEQ ID NO: 102]
TGSSSNIGAGYDVH
CDRL2:
[SEQ ID NO: 103]
SNSQRPS
CDRL3:
[SEQ ID NO: 104]
AAWDDSLNGQVV
Antibody clone: 5D07
5D07-VH
[SEQ ID NO: 12]
EVQLLESGGGLVQPGGSLRLSCAASGFTFSTYGMHWVRQAPGKGLEWVAVIAYDGSKKDYADSVK
GRFTISRDNSKNTLYLQMNSLRAEDTAVYYCAREYRDAFDIWGQGTLVTVSS
5D07-VL
[SEQ ID NO: 36]
QSVLTQPPSASGTPGQRVTISCTGSSSNIGAGYDVHWYQQLPGTAPKLLIYGNSNRPSGVPDRFSG
SKSGTTASLAISGLRSEDEADYYCAAWDDSVSGWMFGGGTKLTVLG
CDR regions
CDRH1:
[SEQ ID NO: 105]
TYGMH
CDRH2:
[SEQ ID NO: 106]
VIAYDGSKKDYADSVKG
CDRH3:
[SEQ ID NO: 107]
EYRDAFDI
CDRL1:
[SEQ ID NO: 108]
TGSSSNIGAGYDVH
CDRL2:
[SEQ ID NO: 109]
GNSNRPS
CDRL3:
[SEQ ID NO: 110]
AAWDDSVSGWM
Antibody clone: 5E12
5E12-VH
[SEQ ID NO: 13]
EVQLLESGGGLVQPGGSLRLSCAASGFTFSSYGMHWVRQAPGKGLEWVAVISYDGINKDYADSMK
GRFTISRDNSKNTLYLQMNSLRAEDTAVYYCARERKDAFDIWGQGTLVTVSS
5E12-VL
[SEQ ID NO: 37]
QSVLTQPPSASGTPGQRVTISCTGSSSNIGAGYDVHWYQQLPGTAPKLLIYSNNQRPSGVPDRFSG
SKSGTSASLAISGLRSEDEADYYCATWDDSLNGLVFGGGTKLTVLG
CDR regions
CDRH1:
[SEQ ID NO: 111]
SYGMH
CDRH2:
[SEQ ID NO: 112]
VISYDGINKDYADSMKG
CDRH3:
[SEQ ID NO: 113]
ERKDAFDI
CDRL1:
[SEQ ID NO: 114]
TGSSSNIGAGYDVH
CDRL2:
[SEQ ID NO: 115]
SNNQRPS
CDRL3:
[SEQ ID NO: 116]
ATWDDSLNGLV
Antibody clone: 5G08
5G08-VH
[SEQ ID NO: 14]
EVQLLESGGGLVQPGGSLRLSCAASGFTFNNYGMHWVRQAPGKGLEWVAVISYDGSNRYYADSVK
GRFTMSRDNSKNTLYLQMNSLRAEDTAVYYCARDRWNGMDVWGQGTLVTVSS
5G08-VL
[SEQ ID NO: 38]
QSVLTQPPSASGTPGQRVTISCSGSSSNIGAGYDVHWYQQLPGTAPKLLIYANNQRPSGVPDRFSG
SKSGTSASLAISGLRSEDEADYYCAAWDDSLNGPWVFGGGTKLTVLG
CDR regions
CDRH1:
[SEQ ID NO: 117]
NYGMH
CDRH2:
[SEQ ID NO: 118]
VISYDGSNRYYADSVKG
CDRH3:
[SEQ ID NO: 119]
DRWNGMDV
CDRL1:
[SEQ ID NO: 120]
SGSSSNIGAGYDVH
CDRL2:
[SEQ ID NO: 121]
ANNQRPS
CDRL3:
[SEQ ID NO: 122]
AAWDDSLNGPWV
Antibody clone: 5H06
5H06-VH
[SEQ ID NO: 15]
EVQLLESGGGLVQPGGSLRLSCAASGFTFSSYGMHWVRQAPGKGLEWVAVISYDGSDTAYADSVK
GRFTISRDNSKNTLYLQMNSLRAEDTAVYYCARDHSVIGAFDIWGQGTLVTVSS
5H06-VL
[SEQ ID NO: 39]
QSVLTQPPSASGTPGQRVTISCSGSSSNIGSNTVNWYQQLPGTAPKLLIYDNNKRPSGVPDRFSGS
KSGTSASLAISGLRSEDEADYYCSSYAGSNNVVFGGGTKLTVLG
CDR regions
CDRH1:
[SEQ ID NO: 123]
SYGMH
CDRH2:
[SEQ ID NO: 124]
VISYDGSDTAYADSVKG
CDRH3:
[SEQ ID NO: 125]
DHSVIGAFDI
CDRL1:
[SEQ ID NO: 126]
SGSSSNIGSNTVN
CDRL2:
[SEQ ID NO: 127]
DNNKRPS
CDRL3:
[SEQ ID NO: 128]
SSYAGSNNVV
Antibody clone: 6A09
6A09-VH
[SEQ ID NO: 16]
EVQLLESGGGLVQPGGSLRLSCAASGFTFSSYGMHWVRQAPGKGLEWVAVTSYDGNTKYYANSVK
GRFTISRDNSKNTLYLQMNSLRAEDTAVYYCAREDCGGDCFDYWGQGTLVTVSS
6A09-VL
[SEQ ID NO: 40]
QSVLTQPPSASGTPGQRVTISCTGSSSNIGAGYDVHWYQQLPGTAPKLLIYGNSNRPSGVPDRFSG
SKSGTSASLAISGLRSEDEADYYCAAWDDSLNEGVFGGGTKLTVLG
CDR regions
CDRH1:
[SEQ ID NO: 129]
SYGMH
CDRH2:
[SEQ ID NO: 130]
VTSYDGNTKYYANSVKG
CDRH3:
[SEQ ID NO: 131]
EDCGGDCFDY
CDRL1:
[SEQ ID NO: 132]
TGSSSNIGAGYDVH
CDRL2:
[SEQ ID NO: 133]
GNSNRPS
CDRL3:
[SEQ ID NO: 134]
AAWDDSLNEGV
Antibody clone: 6B01
6B01-VH
[SEQ ID NO: 17]
EVQLLESGGGLVQPGGSLRLSCAASGFTFSNYGMHWVRQAPGKGLEWVAVISYDGSNKYYADSVK
GRFTISRDNSKNTLYLQMNSLRAEDTAVYYCARDQLGEAFDIWGQGTLVTVSS
6B01-VL
[SEQ ID NO: 41]
QSVLTQPPSASGTPGQRVTISCTGSSSNIGAGYDVHWYQQLPGTAPKLLIYDNNKRPSGVPDRFSG
SKSGTSASLAISGLRSEDEADYYCATWDDSLSGPVFGGGTKLTVLG
CDR regions
CDRH1:
[SEQ ID NO: 135]
NYGMH
CDRH2:
[SEQ ID NO: 136]
VISYDGSNKYYADSVKG
CDRH3:
[SEQ ID NO: 137]
DQLGEAFDI
CDRL1:
[SEQ ID NO: 138]
TGSSSNIGAGYDVH
CDRL2:
[SEQ ID NO: 139]
DNNKRPS
CDRL3:
[SEQ ID NO: 140]
ATWDDSLSGPV
Antibody clone: 6C11
6C11-VH
[SEQ ID NO: 18]
EVQLLESGGGLVQPGGSLRLSCAASGFTFDDYGMSWVRQAPGKGLEWVSAISGSGSSTYYADSVK
GRFTISRDNSKNTLYLQMNSLRAEDTAVYYCAGGDIDYFDYWGQGTLVTVSS
6C11-VL
[SEQ ID NO: 42]
QSVLTQPPSASGTPGQRVTISCTGSSSNFGAGYDVHWYQQLPGTAPKLLIYENNKRPSGVPDRFSG
SKSGTSASLAISGLRSEDEADYYCAAWDDSLNGPVFGGGTKLTVLG
CDR regions
CDRH1:
[SEQ ID NO: 141]
DYGMS
CDRH2:
[SEQ ID NO: 142]
AISGSGSSTYYADSVKG
CDRH3:
[SEQ ID NO: 143]
GDIDYFDY
CDRL1:
[SEQ ID NO: 144]
TGSSSNFGAGYDVH
CDRL2:
[SEQ ID NO: 145]
ENNKRPS
CDRL3:
[SEQ ID NO: 146]
AAWDDSLNGPV
Antibody clone: 6C12
6C12-VH
[SEQ ID NO: 19]
EVQLLESGGGLVQPGGSLRLSCAASGFTFSSYGMHWVRQAPGKGLEWVAVISYDGSNKYYADSVK
GRFTISRDNSKNTLYLQMNSLRAEDTAVYYCARERRDAFDIWGQGTLVTVSS
6C12-VL
[SEQ ID NO: 43]
QSVLTQPPSASGTPGQRVTISCTGSSSNIGAGYDVHWYQQLPGTAPKLLIYSDNQRPSGVPDRFSG
SKSGTSASLAISGLRSEDEADYYCATWDSDTPVFGGGTKLTVLG
CDR regions
CDRH1:
[SEQ ID NO: 147]
SYGMH
CDRH2:
[SEQ ID NO: 148]
VISYDGSNKYYADSVKG
CDRH3:
[SEQ ID NO: 149]
ERRDAFDI
CDRL1:
[SEQ ID NO: 150]
TGSSSNIGAGYDVH
CDRL2:
[SEQ ID NO: 151]
SDNQRPS
CDRL3:
[SEQ ID NO: 152]
VATWDSDTPV
Antibody clone: 6D01
6D01-VH
[SEQ ID NO: 20]
EVQLLESGGGLVQPGGSLRLSCAASGFTFSSYGMHWVRQAPGKGLEWVAVISYDGSNKYYADSVK
GRFTISRDNSKNTLYLQMNSLRAEDTAMYYCARDHSAAGYFDYWGQGTLVTVSS
6D01-VL
[SEQ ID NO: 44]
QSVLTQPPSASGTPGQRVTISCSGSSSNIGSNTVNWYQQLPGTAPKLLIYGNSIRPSGGPDRFSGS
KSGTSASLAISGLRSEDEADYYCASWDDSLSSPVFGGGTKLTVLG
CDR regions
CDRH1:
[SEQ ID NO: 153]
SYGMH
CDRH2:
[SEQ ID NO: 154]
VISYDGSNKYYADSVKG
CDRH3:
[SEQ ID NO: 155]
DHSAAGYFDY
CDRL1:
[SEQ ID NO: 156]
SGSSSNIGSNTVN
CDRL2:
[SEQ ID NO: 157]
GNSIRPS
CDRL3:
[SEQ ID NO: 158]
ASWDDSLSSPV
Antibody clone: 6G03
6G03-VH
[SEQ ID NO: 21]
EVQLLESGGGLVQPGGSLRLSCAASGFTFGSYGMHWVRQAPGKGLEWVSGISWDSAIIDYAGSVK
GRFTISRDNSKNTLYLQMNSLRAEDTAVYYCAKDEAAAGAFDIWGQGTLVTVSS
6G03-VL
[SEQ ID NO: 45]
QSVLTQPPSASGTPGQRVTISCTGSSSNIGAGYDVHWYQQLPGTAPKLLIYGNTDRPSGVPDRFSG
SKSGTSASLAISGLRSEDEADYYCAAWDDSLSGPVVFGGGTKLTVLG
CDR regions
CDRH1:
[SEQ ID NO: 159]
SYGMH
CDRH2:
[SEQ ID NO: 160]
GISWDSAIIDYAGSVKG
CDRH3:
[SEQ ID NO: 161]
DEAAAGAFDI
CDRL1:
[SEQ ID NO: 162]
TGSSSNIGAGYDVH
CDRL2:
[SEQ ID NO: 163]
GNTDRPS
CDRL3:
[SEQ ID NO: 164]
AAWDDSLSGPVV
Antibody clone: 6G08
6G08-VH
[SEQ ID NO: 22]
EVQLLESGGGLVQPGGSLRLSCAASGFTLSSYGISWVRQAPGKGLEWVSGISGSGGNTYYADSVK
GRFTISRDNSKNTLYLQMNSLRAEDTAVYYCASSVGAYANDAFDIWGQGTLVTVSS
6G08-VL
[SEQ ID NO: 46]
QSVLTQPPSASGTPGQRVTISCTGSSSNIGAGYDVHWYQQLPGTAPKLLIYGDTNRPSGVPDRFSG
SKSGTSASLAISGLRSEDEADYYCAAWDDSLNGPVFGGGTKLTVLG
CDR regions
CDRH1:
[SEQ ID NO: 165]
SYGIS
CDRH2:
[SEQ ID NO: 166]
GISGSGGNTYYADSVKG
CDRH3:
[SEQ ID NO: 167]
SVGAYANDAFDI
CDRL1:
[SEQ ID NO: 168]
TGSSSNIGAGYDVH
CDRL2:
[SEQ ID NO: 169]
GDTNRPS
CDRL3:
[SEQ ID NO: 170]
AAWDDSLNGPV
Antibody clone: 6G11
6G11-VH
[SEQ ID NO: 23]
EVQLLESGGGLVQPGGSLRLSCAASGFTFSSYGMHWVRQAPGKGLEWMAVISYDGSNKYYADSVK
GRFTISRDNSKNTLYLQMNSLRAEDTAVYYCARELYDAFDIWGQGTLVTVSS
6G11-VL
[SEQ ID NO: 47]
QSVLTQPPSASGTPGQRVTISCTGSSSNIGAGYDVHWYQQLPGTAPKLLIYADDHRPSGVPDRFSG
SKSGTSASLAISGLRSEDEADYYCASWDDSQRAVIFGGGTKLTVLG
CDR regions
CDRH1:
[SEQ ID NO: 171]
SYGMH
CDRH2:
[SEQ ID NO: 172]
VISYDGSNKYYADSVKG
CDRH3:
[SEQ ID NO: 173]
ELYDAFDI
CDRL1:
[SEQ ID NO: 174]
TGSSSNIGAGYDVH
CDRL2:
[SEQ ID NO: 175]
ADDHRPS
CDRL3:
[SEQ ID NO: 176]
ASWDDSQRAVI
Antibody clone: 6H08
6H08-VH
[SEQ ID NO: 24]
EVQLLESGGGLVQPGGSLRLSCAASGFTFNNYGMHWVRQAPGKGLEWVAVISYDGSNKYYADSVK
GRFTISKDNSKNTLYLQMNSLRAEDTAVYYCAREYKDAFDIWGQGTLVTVSS
6H08-VL
[SEQ ID NO: 48]
QSVLTQPPSASGTPGQRVTISCTGSSSNIGSNTVNWYQQLPGTAPKLLIYDNNKRPSGVPDRFSGS
KSGTSASLAISGLRSEDEADYYCQAWGTGIRVFGGGTKLTVLG
CDR regions
CDRH1:
[SEQ ID NO: 177]
NYGMH
CDRH2:
[SEQ ID NO: 178]
VISYDGSNKYYADSVKG
CDRH3:
[SEQ ID NO: 179]
EYKDAFDI
CDRL1:
[SEQ ID NO: 180]
TGSSSNIGSNTVN
CDRL2:
[SEQ ID NO: 181]
DNNKRPS
CDRL3:
[SEQ ID NO: 182]
QAWGTGIRV
Antibody clone: 7C07
7C07-VH
[SEQ ID NO: 25]
EVQLLESGGGLVQPGGSLRLSCAASGFTFSSYGMHWVRQAPGKGLEWVAVISYDGSNKYYADSVK
GRFTISRDNSQNTLYLQMNSLRAEDTAVYYCAREFGYIILDYWGQGTLVTVSS
7C07-VL
[SEQ ID NO: 49]
QSVLTQPPSASGTPGQRVTISCSGSSSNIGSNTVNWYQQLPGTAPKLLIYRDYERPSGVPDRFSGS
KSGTSASLAISGLRSEDEADYYCMAWDDSLSGVVFGGGTKLTVLG
CDR regions
CDRH1:
[SEQ ID NO: 183]
SYGMH
CDRH2:
[SEQ ID NO: 184]
VISYDGSNKYYADSVKG
CDRH3:
[SEQ ID NO: 185]
EFGYIILDY
CDRL1:
[SEQ ID NO: 186]
SGSSSNIGSNTVN
CDRL2:
[SEQ ID NO: 187]
RDYERPS
CDRL3:
[SEQ ID NO: 188]
MAWDDSLSGVV
Antibody clone: 4B02
4B02-VH
[SEQ ID NO: 26]
EVQLLESGGGLVQPGGSLRLSCAASGFTFSNHGMHWVRQAPGKGLEWVAVISYDGTNKYYADSVR
GRFTISRDNSKNTLYLQMNSLRAEDTAVYYCARETWDAFDVWGQGTLVTVSS
4B02-VL
[SEQ ID NO: 50]
QSVLTQPPSASGTPGQRVTISCSGSSSNIGSNNANWYQQLPGTAPKLLIYDNNKRPSGVPDRFSGS
KSGTSASLAISGLRSEDEADYYCQAWDSSTVVFGGGTKLTVLG
CDR regions
CDRH1:
[SEQ ID NO: 189]
NHGMH
CDRH2:
[SEQ ID NO: 190]
VISYDGTNKYYADSVRG
CDRH3:
[SEQ ID NO: 191]
ETWDAFDV
CDRL1:
[SEQ ID NO: 192]
SGSSSNIGSNNAN
CDRL2:
[SEQ ID NO: 193]
DNNKRPS
CDRL3:
[SEQ ID NO: 194]
QAWDSSTVV

In some embodiments the antibody molecule that specifically binds FcγRIIb is a human antibody.

In some embodiments, the antibody molecule that specifically binds FcγRIIb is an antibody of human origin, i.e. an originally human antibody that has been modified as described herein.

In some embodiments, the antibody molecule that specifically binds FcγRIIb is a humanized antibody, i.e. an originally non-human antibody that has been modified to increase its similarity to a human antibody. The humanized antibodies may, for example, be murine antibodies or llama antibodies.

As discussed above, the first antibody may be a monoclonal antibody or an antibody molecule of monoclonal origin.

It is well known that an antibody specifically binds to or interacts with a defined target molecule or antigen. That is to say, the antibody preferentially and selectively binds its target and not a molecule which is not a target.

Methods of assessing protein binding are known to the person skilled in biochemistry and immunology. It would be appreciated by the skilled person that those methods could be used to assess binding of an antibody to a target and/or binding of the Fc region of an antibody to an Fc receptor; as well as the relative strength, or the specificity, or the inhibition, or prevention, or reduction in those interactions. Examples of methods that may be used to assess protein binding are, for example, immunoassays, BIAcore, western blots, radioimmunoassay (RIA) and enzyme-linked immunosorbent assays (ELISAs) (See Fundamental Immunology Second Edition, Raven Press, New York at pages 332-336 (1989) for a discussion regarding antibody specificity).

Accordingly, by “antibody molecule that specifically binds” we include that the antibody molecule specifically binds a target but does not bind to non-target, or binds to a non-target more weakly (such as with a lower affinity) than the target.

We also include the meaning that the antibody specifically binds to the target at least two-fold more strongly, or at least five-fold more strongly, or at least 10-fold more strongly, or at least 20-fold more strongly, or at least 50-fold more strongly, or at least 100-fold more strongly, or at least 200-fold more strongly, or at least 500-fold more strongly, or at least than about 1000-fold more strongly than to a non-target.

Additionally, we include the meaning that the antibody specifically binds to the target if it binds to the target with a K_{d} of at least about 10⁻¹ K_{d}, or at least about 10⁻² K_{d}, or at least about 10⁻³ K_{d}, or at least about 10⁻⁴ K_{d}, or at least about 10⁻⁵ K_{d}, or at least about 10⁻⁶ K_{d}, or at least about 10⁻⁷ K_{d}, or at least about 10⁻⁸ K_{d}, or at least about 10⁻⁹ K_{d}, or at least about 10⁻¹⁰ K_{d}, or at least about 10⁻¹¹ K_{d}, or at least about 10⁻¹² K_{d}, or at least about 10⁻¹³ K_{d}, or at least about 10⁻¹⁴ K_{d}, or at least about 10⁻¹⁵ K_{d}.

As discussed above, the system, combination, method or use of the present invention is for improving tolerability of an antibody molecule that specifically binds to FcγRIIb in a subject. It is well known that the administration of therapeutic antibodies may be associated with tolerability issues. In some embodiments, these issues may be associated with intravenous administration of said antibody.

The term “tolerability” as used herein refers to the degree to which adverse effects of a therapeutic agent can be tolerated by a subject. By “adverse effect” we include any effect caused by the therapeutic agent, either directly or indirectly, that is not the desired therapeutic effect, or any other beneficial effect attributable to the therapeutic agent, either directly or indirectly.

By “improving tolerability” we include preventing or mitigating tolerability issues associated with administration of the antibody molecule. In another definition, we include reducing or preventing adverse effects associated with administration of the antibody molecule.

The term “tolerability issue” as used herein encompasses different types of adverse effects that may occur in connection with administration, and in particular intravenous administration, of the antibody molecule to a human. These may be, for example, infusion related reactions (IRRs), cytokine release syndrome, thrombocytopenia, hepatic toxicities such as elevated liver enzymes, fever, hypotension and/or dermatological toxicities, including rashes such as urticaria. Herein these different tolerability issues are defined in the way they are defined in the Common Terminology Criteria for Adverse Events (CTCAE) version 5.0 (published by the US Department of Health and Human Services, 27 Nov. 2017), as further described below.

Tolerability issues may be of different grades, i.e. of different severity for the subjects experiencing them. In some cases, they lead to discomfort for the subject, while in others they may cause severe problems that may prevent continued treatment with the therapeutic antibody molecule. In worse cases, the tolerability issues may even lead to death of the subject.

The tolerability issues that may be predicted, prevented and/or mitigated as described herein are adverse events that occur in connection with intravenous administration of the therapeutic antibody molecule to a subject, i.e. immediately when the therapeutic antibody molecule is administered, such as within a few minutes up to a few hours or within 24 hours from administration of the therapeutic antibody molecule to the subject. In many cases, the first tolerability issues are observed within less 30 minutes.

In some preferred embodiments, it is of particular interest to improve the tolerability of the antibody that specifically binds to FcγRIIb specifically in relation to IRRs. In some embodiments, these antibodies may be more likely to cause or lead to IRRs of varying severities in a human subject. It is therefore advantageous to prevent or mitigate such IRRs, as they improve the experience of the subject and also allow the therapeutic antibody to be administered for longer and at higher doses before needing to stop treatment due to tolerability issues (if this is needed at all).

In some cases, it may be of interest to prevent in particular thrombocytopenia and/or hepatic toxicities.

The IRR that may be prevented or mitigated as described herein and/or that may be predicted with the method described herein may be any IRR. The adverse event denoted “Infusion related reaction” in the CTCAE version 5.0 is used for disorders characterized by adverse reaction to the infusion of pharmacological or biological substances; belonging to the group of “Injury, poisoning and procedural complications”. The five grades identified in the CTCAE are the following:
1) Mild transient reaction; infusion interruption not indicated; intervention not indicated
2) Therapy or infusion interruption indicated but responds promptly to symptomatic treatment (e.g., antihistamines, NSAIDS, narcotics, IV fluids); prophylactic medications indicated for hours
3) Prolonged (e.g., not rapidly responsive to symptomatic medication and/or brief interruption of infusion); recurrence of symptoms following initial improvement; hospitalization indicated for clinical sequelae
4) Life-threatening consequences; urgent intervention indicated
5) Death.

Based on the above classifiers, the person skilled in the art would be able to identify an infusion related reaction in a subject following administration of the antibody molecule defined herein, for example by observing a subject for the symptoms of an IRR. These may include, in some embodiments, pruritus, urticaria, fever, rigors/chills, diaphoresis, bronchospasms, nausea, muscle pain, and cardiovascular collapse

In some preferred embodiments, IRRs associated with the antibody molecule described herein are reduced or completely prevented by the dosage regimen described herein.

The adverse event denoted “Cytokine release syndrome” in the CTCAE version 5.0 is used for disorders characterized by fever, tachypnea, headache, tachycardia, hypotension, rash, and/or hypoxia caused by the release of cytokines, belonging to the group of “Immune system disorders”. The five grades identified in the CTCAE are the following:
1) Fever with or without constitutional symptoms
2) Hypotension responding to fluids; hypoxia responding to <40% O₂
3) Hypotension managed with one pressor; hypoxia requiring 40% O₂
4) Life-threatening consequences; urgent intervention indicated
5) Death

The adverse event denoted “Platelet count decreased” (i.e. thrombocytopenia) in the CTCAE version 5.0 is used for findings based on laboratory test results that indicate a decrease in number of platelets in a blood specimen, belonging to the group of “Investigations”. The five grades identified in the CTCAE are the following:
1) <LLN−75,000/mm3; <LLN−75.0×10e9/L
2) <75,000 −50,000/mm3; <75.0 −50.0×10e9/L
3) <50,000 −25,000/mm3; <50.0 −25.0×10e9/L
4) <25,000/mm3; <25.0×10e9/L
5) -

The associated toxicity may also be a hepatic adverse event or hepatic toxicities. Examples of such toxicities are an elevation of one or both of the two enzymes aspartate aminotransferase (AST) and alanine aminotransferase (ALT). Like thrombocytopenia, the adverse events denoted “Aspartate aminotransferase increased” and “Alanine aminotransferase increased” in the CTCAE version 5.0 belong to the group of “Investigations”. Increased AST or ALT, respectively, is a finding based on laboratory test results that indicate an increase in the level of AST (or SGOT) and ALT (or SGPT), respectively, in a blood specimen. The five grades identified in the CTCAE both for increased AST and increased ALT are the following:
1) >ULN−3.0×ULN if baseline was normal; 1.5 −3.0×baseline if baseline was abnormal
2) >3.0 −5.0×ULN if baseline was normal; >3.0 −5.0×baseline if baseline was abnormal
3) >5.0 −20.0×ULN if baseline was normal; >5.0 −20.0×baseline if baseline was abnormal
4) >20.0×ULN if baseline was normal; >20.0×baseline if baseline was abnormal
5) -.

The adverse event denoted “Fever” in the CTCAE version 5.0 is used for disorders characterized by elevation of the body's temperature above the upper limit of normal, belonging to the group of “General disorders and administration site conditions”. The five grades identified in the CTCAE are the following:
1) 38.0-39.0° C.
2) >39.0-40.0° C.
3) >40.0° C. for hours
4) >40.0° C. for >24 hours
5) Death.

The adverse event denoted “Hypotension” in the CTCAE version 5.0 is used for disorders characterized by a blood pressure that is below the normal expected for an individual in a given environment, belonging to the group of “Vascular disorders”. The five grades identified in the CTCAE are the following:
1) Asymptomatic, intervention not indicated
2) Non-urgent medical intervention indicated
3) Medical intervention indicated; hospitalization indicated
4) Life-threatening consequences and urgent intervention indicated
5) Death.

The adverse event denoted “Urticaria” in the CTCAE version 5.0 is used for disorders characterized by an itchy skin eruption characterized by wheals with pale interiors and well-defined red margins, belonging to the group of “Skin and subcutaneous tissue disorders”. The five grades identified in the CTCAE are the following:
1) Urticarial lesions covering <10% BSA; topical intervention indicated
2) Urticarial lesions covering 10-30% BSA; oral intervention indicated
3) Urticarial lesions covering >30% BSA; IV intervention indicated
4)
5) -.

In some other embodiments, the improvement in tolerability is associated with the reduction or prevention of adverse effects observed upon administration of the antibody. These may be effects attributed either directly or indirectly to administration of the antibody, in some embodiments.

In some embodiments, the above mentioned tolerability issues and/or adverse effects cause changes in several subject observations outside of normal levels. In some embodiments, these observations include one or more of the following: body temperature; platelet count; blood levels of liver enzymes (e.g. alanine aminotransferase (ALAT) and/or aspartate aminotransferase (ASAT)); blood levels of cytokines (e.g. IL-6, TNF-α, IL-8, IFN-γ, MIP-1β, IL-10, IL-4, IL-1b, IL-2, IL-12).

The normal levels of each of the above measurements are typically defined as follows:
Body temperature: from 36.1° C. to 37.9° C.;
Platelet count: from 145×10⁹ to 400×10⁹ per litre;
Blood level of ALAT: from 0 to 1.09 μkat/L, 16-63 U/L;
Blood level of ASAT: from 0 to 0.759 μkat/L, 15-37 U/L;
Blood level IL-6: from 0.16 to 27.2 pg/ml, with a median value of 0.47 pg/ml.

In some embodiments, the system, combination, method or use of the present invention reduces changes in each of the above parameters. By “reduces changes”, we mean that the degree of change in each of the above measurements is less when the therapeutic system or dosage regime of the invention is used, compared to when a single dose, equivalent to the sum of the first and second doses (in mg) of the invention as defined herein, of the antibody molecule is administered. Preferably these changes are reduced to within acceptable levels.

By “acceptable levels” we mean that the above measurements, after treatment with the second dose of the antibody molecule, remain within the normal ranges as defined above. In some embodiments, the values of the above measurements remain within the normal ranges defined above after administration of the second dose of the antibody molecule. In some other embodiments, by “acceptable levels” we include that the clinical grading of the IRR (as defined in the art and herein using the CTCAE scale) is reduced to at least grade 2. In some preferred embodiments, the grading of the IRR is reduced to grade 1. As discussed herein, the skilled person will be aware of how to grade an IRR according to the CTCAE scale.

In some preferred embodiments, these values remain within normal levels, or are changed within acceptable levels, for at least 24 hours after administration of the second dose of the antibody molecule.

As discussed above, the invention provides a system, combination, method, or use, in which a corticosteroid is administered to the subject before the first dose of the antibody molecule. Corticosteroids are a well-known class of steroid hormones that have been used for a wide variety of clinical applications.

As shown in Example 1, corticosteroids have surprisingly been found to provide a protective effect against infusion related reactions associated with administration of the therapeutic antibody of the invention. As also shown in Example 2, other compounds that have been previously used to treat IRRs in the clinic did not provide a protective effect (or simply provided an additive effect). These other compounds that have been commonly used to treat IRRs include but are not limited to the following: anti-histamines (e.g. H1 and H2 blockers), anti-PAF, anti-IL-6R, and a leukotriene receptor antagonist (e.g. montelukast). This renders the protective effect of corticosterioids alone surprising in the context of the present invention, as none of these other commonly used therapies provided a similar protective effect.

In preferred embodiments of the system, combination, method or use of the present invention, the corticosteroid is administered to the subject at a time point from 10 minutes to 48 hours before the first dose of the antibody molecule that specifically binds to FcγRIIb. More preferably, the corticosteroid is administered to the subject at a time point from 10 minutes to 24 hours before the first dose of the antibody molecule that specifically binds to FcγRIIb.

Thus, in embodiments of the invention, the corticosteroid is administered at a time point of about 10 minutes, or about 20 minutes, or about 30 minutes, or about 40 minutes, or about 50 minutes, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, before the first dose of the antibody molecule that specifically binds to FcγRIIb.

In embodiments of the invention, the corticosteroid may be administered in more than one dose before the first dose of the antibody molecule that binds specifically to FcγRIIb. For instance, the corticosteroid may be administered in two doses, three doses, four doses, five doses, six doses, seven doses, eight doses, nine doses, ten doses, eleven doses, twelve doses, or more than twelve doses, before the first dose of the antibody molecule that binds specifically to FcγRIIb.

In some additional or alternative embodiments, when more than one dose of the corticosteroid is administered, the corticosterioid may be administered both before and after the first dose of the antibody molecule that binds specifically to FcγRIIb (but before the second dose of the antibody that binds specifically to FcγRIIb). At least one dose of the corticosterioid will be administered before the first dose of the antibody molecule, but the other, subsequent, corticosteroid doses described will be administered after the first dose of the antibody molecule, and may be distributed between the antibody doses in any order.

In these embodiments, the corticosteroid administration before the second dose of the antibody that binds specifically to FcγRIIb may be at a time point about 10 minutes, or about 20 minutes, or about 30 minutes, or about 40 minutes, or about 50 minutes, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, before the second dose of the antibody molecule that specifically binds to FcγRIIb.

Preferably, the corticosteroid is administered as a first dose and a second dose, before the first dose of the antibody that binds specifically to FcγRIIb. Preferably, when the corticosteroid is administered as a first dose and a second dose, the first dose of the corticosteroid is administered at a time point from 16 hours to 48 hours before the first dose of the antibody molecule that specifically binds to FcγRIIb, and the second dose of the corticosteroid is administered at a time point from 10 minutes to 2 hours before the first dose of the antibody molecule that binds specifically to FcγRIIb.

It will be appreciated that, in such embodiments of the invention, the first dose of the corticosteroid may be administered at any time point between 16 hours to 48 hours before the first dose of the antibody molecule—for example, at a time point of about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, before the first dose of the antibody molecule that specifically binds to FcγRIIb. It will also be appreciated that, in such embodiments of the invention, the second dose of the corticosteroid may be administered at any time point between 10 minutes to 2 hours before the first dose of the antibody molecule—for example, at a time point of about 10 minutes, or about 20 minutes, or about 30 minutes, or about 40 minutes, or about 50 minutes, or about 1 hour, or about 2 hours, before the first dose of the antibody molecule that specifically binds to FcγRIIb.

In further preferred embodiments of the invention, a further dose of corticosteroid is administered before the second dose of antibody molecule that specifically binds to FcγRIIb. Thus, in such embodiments, the further dose of corticosteroid is administered after the first dose of the antibody molecule but before the second dose of the antibody molecule. Preferably, one or more further dose of corticosteroid is administered—such as one further dose; or two further doses; or three further doses; or four further doses; or five further doses; or six further doses; or seven further doses; or eight further doses; or nine further doses; or ten further doses; or eleven further doses; or twelve further doses, or more.

Preferably, the further dose of corticosteroid is administered at a time point from 16 hours to 48 hours before the second dose of the antibody molecule that specifically binds to FcγRIIb. Accordingly, in such embodiments of the invention, the further dose of the corticosteroid may be administered at any time point between 16 hours to 48 hours before the second dose of the antibody molecule—for example, at a time point of about 16 hours, or about 17 hours, or about 18 hours, or about 19 hours, or about 20 hours, or about 21 hours, or about 22 hours, or about 23 hours, or about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, before the second dose of the antibody molecule that specifically binds to FcγRIIb.

In some embodiments, the dosage regimens described herein can be repeated as many times as necessary in a particular patient. For instance, this dosage regimen can be employed each and every time the antibody molecule that specifically binds to FcγRIIb is administered to the patient. In some embodiments, the exact format of the dosage regimen (in terms of timing and amounts of doses) may be varied between repeat administrations to the patient. The advantage of using the dosage regimens described herein repeatedly is that it ensures that the improved tolerability (for example a reduction in infusion related reactions) is achieved with each administration of the antibody that specifically binds FcγRIIb.

Corticosterioids of the invention can be administered at a dose of from 0.5 to 20 mg. In preferred embodiments of the invention, the corticosteroid is administered at a dose of from about 4 mg to about 20 mg, such as at a dose of from about 12 mg to about 20 mg, or at a dose of from about 4 mg to about 12 mg. For example, the corticosteroid is administered at a dose of about 4 mg or greater, such as at about 5 mg or greater, or about 6 mg or greater, or about 7 mg or greater, or about 8 mg or greater, or about 9 mg or greater, or about 10 mg or greater, or about 11 mg or greater, or about 12 mg or greater, or about 13 mg or greater, or about 14 mg or greater, or about 15 mg or greater, or about 16 mg or greater, or about 17 mg or greater, or about 18 mg or greater, or about 19 mg or greater, or about 20 mg or greater.

In some preferred embodiments, the corticosteroid is dexamethasone. In some additional or alternative embodiments, the corticosteroid is betamethasone. In some embodiments, a combination of dexamethasone and betamethasone is used. A skilled person will appreciate that other corticosteroids are contemplated by the present invention, for example, one or more of the following: cortisone; hydrocortisone; prednisone; prednisolone; triamcinolone; and methylprednisolone; or combinations thereof.

In some embodiments, when the corticosteroid is dexamethasone, the dose of dexamethasone is from 0.5 mg to 20 mg. In some embodiments, when dexamethasone is used, the dose of dexamethasone is about 4 mg or greater, such as about 4-20 mg in a preferred embodiment. In some embodiments, the dose of dexamethasone is about 12 mg or greater, such as about 12-20 mg. In some embodiments, the dose of dexamethasone is about 4-12 mg. In particularly preferred embodiments, the dose of dexamethasone is about about 12 mg or is about about 20 mg.

In particularly preferred embodiments of the invention, a first dose and a second dose of the corticosteroid dexamethasone is administered. More preferably in these embodiments of the invention when dexamethasone is used: the first dose is about 4-20 mg and/or the second dose is about 4-25 mg; or the first dose is about 4-20 mg and second dose is about 4-25 mg; or the first dose is about 10⁻¹² mg and/or the second dose is about 20 mg; or the first dose is about 10⁻¹² mg and the second dose is about 20 mg.

In some embodiments, when the corticosteroid is betamethasone, the dose of betamethasone is from 0.5 mg to 20 mg. In some embodiments, when betamethasone is used, the dose of betamethasone is about 3.2 mg or greater, such as about 4 mg or greater, such as about 3.2-16 mg, or about 4-20 mg. In some embodiments, the dose of betamethasone is about 12 mg or greater, such as about 12-20 mg. In some embodiments, the dose of betamethasone is about 4-12 mg. In particularly preferred embodiments, the dose of betamethasone is about about 12 mg or is about about 20 mg.

In particularly preferred embodiments of the invention, a first dose and a second dose of the corticosteroid betamethasone is administered. More preferably in these embodiments of the invention when betamethasone is used: the first dose is about 3.2-16 mg and/or the second dose is about 3.2-20 mg; or the first dose is about 3.2-16 mg and second dose is about 3.2-20 mg; or the first dose is about 8-9.6 mg and/or the second dose is about 16 mg; or the first dose is about 8-9.6 mg and the second dose is about 16 mg.

A skilled person will appreciate that other corticosteroids are known in the art, in addition to those described herein; as corticosteroids function in a similar manner, it will be appreciated that any corticosteroid could be used in the present invention.

As discussed above, the invention provides a system, combination, method, or use, in which an antibody molecule that specifically binds to FcγRIIb is administered to the subject as at least a first dose and a second dose.

In preferred embodiments of the invention, the first dose of the antibody molecule that specifically binds to FcγRIIb is administered at a time point from about one to about 24 hours before the second dose of the antibody molecule that specifically binds to FcγRIIb. Accordingly, in such embodiments of the invention, the first dose of the antibody molecule is administered at any time point between about one to about 24 hours before the second dose of the antibody molecule—for example, at a time point of about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, or about 13 hours, or about 14 hours, or about 15 hours, or about 16 hours, or about 17 hours, or about 18 hours, or about 19 hours, or about 20 hours, or about 21 hours, or about 22 hours, or about 23 hours, or about 24 hours, before the second dose of the antibody molecule that specifically binds to FcγRIIb.

Most preferably in that embodiment of the invention, the first dose of the antibody molecule that specifically binds to FcγRIIb is administered about one hour before the second dose of the antibody molecule that specifically binds to FcγRIIb, or is administered about 24 hours before the second dose of the antibody molecule that specifically binds to FcγRIIb.

In another embodiment, the first dose of the antibody molecule that specifically binds to FcγRIIb is administered at a time point from about 24 hours to about 48 hours before the second dose of the antibody molecule that specifically binds to FcγRIIb. Accordingly, in such embodiments of the invention, the first dose of the antibody molecule is administered at any time point between about 24 hours to about 48 hours before the second dose of the antibody molecule—for example, at a time point of about 24 hours, or about 25 hours, or about 26 hours, or about 27 hours, or about 28 hours, or about 29 hours, or about 30 hours, or about 31 hours, or about 32 hours, or about 33 hours, or about 34 hours, or about 35 hours, or about 36 hours, or about 37 hours, or about 38 hours, or about 39 hours, or about 40 hours, or about 41 hours, or about 42 hours, or about 43 hours, or about 44 hours, or about 45 hours, or about 46 hours, or about 47 hours, or about 48 hours, before the second dose of the antibody molecule that specifically binds to FcγRIIb.

As discussed above, the invention provides a system, combination, method, or use, in which the first dose of the antibody molecule is lower than the maximum therapeutically effective dose of the antibody molecule.

Those skilled is the art will be aware that for approved antibody therapies, certain doses (typically expressed in mg/kg) are recommended for use in certain patient groups or for subjects with a particular type of cancer. Often, recommended doses are described in the labelling or prescription information of an approved antibody therapeutic. The recommended dose may be calculated for a particular subject, i.e. based on the type of cancer, the stage of the cancer, their weight, Body Mass Index (BMI) and other factors.

The skilled person will appreciate that the recommended dose will differ depending on the identity of the antibody molecule. Where the antibody molecule is not described in labelling or prescription information, it would be apparent to the skilled person how to determine the recommended dose using techniques well known in the art.

The “recommended dose” is typically referred to as the “approved dose”, “maximal tolerated dose (MTD)” or the “therapeutically effective dose” of an antibody molecule. The MTD is a well-recognised term in drug development and refers to the highest dose of the drug that can be used with an acceptable level of tolerability.

By “therapeutically effective dose” we mean any dose that would be considered to be therapeutically active (i.e. which produces the desired therapeutic effect in a subject defined herein).

By the “maximum therapeutically effective dose”, we mean the (lowest) dose that achieves maximal therapeutic activity, without consideration of tolerability (which may be suboptimal or not tolerated in absence of appropriate measures of administration to mitigate adverse effects). This is the ideal dosage that will be attempted to be used by those skilled in the art when administering the antibody molecule to a subject in need thereof.

By “therapeutically active” we include where the dose produces the desired therapeutic effect in a subject. By “therapeutic effect” we include all effects that are attributable directly or indirectly to use of the therapy in question. This may be a measurable therapeutic effect, such as reduced tumour volume or reduced tumour size (which may be determined by a CT scan, for example), or effectiveness of a therapeutic antibody or treatment. In other cases, this may be a more subjective effect, such as a reduction in severity of symptoms reported by the subject. The measurement of therapeutic effects in subjects in response to the administration of therapeutic antibodies is well known in the art. Furthermore, the level of survival of a subject or group of subjects over a defined time period is an alternative read-out of therapeutic effect.

The present invention is based on the inventors' surprising discovery that tolerability of an antibody molecule that specifically binds to FcγRIIb in a subject is improved when the subject is administered a corticosteroid, and is subsequently administered the antibody molecule in at least a first dose and a second dose, in which the first dose is lower than the “maximum therapeutically effective dose” of that antibody. Put another way, the first dose of the antibody molecule is a sub-maximal therapeutic dose—that is, it is a lower dose than the maximum therapeutically effective dose of the antibody.

In preferred embodiments of the invention, the first dose of the antibody molecule that specifically binds to FcγRIIb is lower than the maximum tolerated therapeutic dose. As discussed above, the maximum tolerated therapeutic dose is the highest dose of the drug that can be used that is considered to be tolerated (i.e. does not produce unacceptable levels of toxicity or side effects in the patient, and this may be lower than the maximum therapeutically effective dose. This differs from the maximum therapeutically effective dose in that the dose must be tolerated in the patient. The level of side-effects/toxicity that can be tolerated by a particular patient depend on factors such as the stage or severity of disease.

Improving drug tolerability and the therapeutic window is not only important for treatment of severely ill patients e.g. those with cancer, but can be critical for use in patients with non-life-threatening disease e.g. autoimmune or infectious diseases where moderate or even mild side-effects may not be acceptable.

In some other cases, the dose that is lower than the maximum therapeutically effective dose or the maximum tolerated therapeutic dose is lower than the lowest dose thought to be therapeutically effective (i.e. the minimum effective dose). In other words, the first dose of the antibody molecule may be a dose that would not be therapeutically effective when administered alone as a single dose.

In some other cases, the first dose of the antibody is lower than the maximum feasible dose. In some cases practicalities, such as formulation considerations, may limit the maximum dose that can be administered. The maximum such dose taking into account such factors is termed the maximum feasible dose.

In some other cases, the dose that is lower than the tolerated therapeutic dose is lower than the recommended tolerated therapeutic dose. In some embodiments, this may include the recommended dose for the indication included in the drug label.

It will be apparent to the skilled person in the art how the particular tolerated therapeutic dose is defined for any particular antibody, generally using dose escalation studies during clinical trials. Tolerated therapeutic doses for antibodies that have not yet been approved may be based on the tolerated therapeutic doses of similar antibodies that have been approved or have undergone extensive clinical testing.

In preferred embodiments of the present invention, the first dose of the antibody molecule that specifically binds to FcγRIIb is at least 50% lower than the maximum therapeutically effective dose. For example, the first dose of the antibody molecule is at least 60% lower, or at least 70% lower, or at least 80% lower, or at least 90% lower than the maximum therapeutically effective dose.

In one embodiment of the invention, the antibody molecule that specifically binds to FcγRIIb is administered in a first dose that results in high receptor saturation of the FcγRIIb receptor, such as: at least 50% receptor saturation; or at least 60% receptor saturation; or at least 70% receptor saturation; or at least 80% receptor saturation; or at least 90% receptor saturation; or at least 95% receptor saturation; or at least 96% receptor saturation; or at least 97% receptor saturation; or at least 98% receptor saturation; or at least 99% receptor saturation; or close to 100% receptor saturation; or 100% receptor saturation as measured at a time-point between end of infusion and up to immediately before the second antibody infusion. Methods for measuring receptor saturation are well known to those skilled in the art.

Preferably, the high receptor saturation is at least transient, but may be maintained for longer periods. By transient receptor saturation we mean indicated saturation is maintained for at least 15 min, and preferably 1 to 6 hours, and most preferably lasting until the second antibody administration. As discussed herein, the time period between the first and second antibody molecule doses may vary from between about 1 hour to about 48 hours.

As discussed herein, the dose of an antibody molecule can be expressed based on the weight of subject to whom it is to be administered—typically in mg of the antibody molecule per kg weight of the subject.

In preferred embodiments of the present invention, the first dose of the antibody molecule that specifically binds to FcγRIIb is administered at a dose of from about 0.2 mg/kg to about 0.6 mg/kg; for example, at a dose of from about 0.3 mg/kg to about 0.5 mg/kg. It will therefore be appreciated that the first dose of the antibody molecule may be administered at a dose of: about 0.2 mg/kg, or at about 0.3 mg/kg, or at about 0.4 mg/kg, or at about 0.5 mg/kg, or at about 0.6 mg/kg.

It will be appreciated, that depending on the weight of the subject to whom the antibody molecule is to be administered, the first dose of the antibody molecule that specifically binds to FcγRIIb may be administered at a dose of from about 10 mg to about 20 mg. In other embodiments, the first dose of the antibody may be administered at a dose of from about 20 mg to about 40 mg, or more; for example, at a dose of from about 20 mg to 30 mg, or from about 30 mg to 40 mg, or from about 40 mg to 50 mg, or from about 50 mg to 60 mg, or from about 60 mg to 70 mg, or more. It will therefore be appreciated that the first dose of the antibody molecule may be administered at a dose of: about 10 mg, about 20 mg, or about 25 mg, or about 30 mg, or about 35 mg, or about 40 mg, or about 45 mg, or about 50 mg, or about 55 mg, or about 60 mg, or about 65 mg, or about 70 mg, or more.

In one embodiment of the invention, the antibody molecule that specifically binds to FcγRIIb is administered in a dose that results in high receptor saturation, at least transiently, of the FcγRIIb receptor, such as: at least 50% receptor saturation; or at least 60% receptor saturation; or at least 70% receptor saturation; or at least 80% receptor saturation; or at least 90% receptor saturation; or at least 95% receptor saturation; or at least 96% receptor saturation; or at least 97% receptor saturation; or at least 98% receptor saturation; or at least 99% receptor saturation; or close to 100% receptor saturation; or 100% receptor saturation. Methods for measuring receptor saturation are well known to those skilled in the art.

By transient receptor saturation we mean indicated saturation is maintained for at least 15 min, and preferably 1 to 6 hours, and most preferably lasting until the second antibody administration.

As discussed above, the invention provides a system, combination, method, or use, in which a second dose of the antibody molecule that specifically binds to FcγRIIb is administered to the subject.

Preferably, the second dose of the antibody molecule that specifically binds to FcγRIIb is a therapeutically effective dose. In some embodiments, the second dose of the antibody molecule may be the maximum therapeutically effective dose as defined herein.

More preferably, the second dose of the antibody molecule that specifically binds to FcγRIIb is the maximum tolerated therapeutic dose or the maximum feasible therapeutic dose.

More preferably, the second dose of the antibody molecule that specifically binds to FcγRIIb is lower than a therapeutically effective dose.

In some embodiments, the second dose of the antibody molecule is higher than the first dose of the antibody molecule. In alternative embodiments, the second dose of the antibody molecule is lower than the first dose of the antibody molecule.

In some embodiments, the total amount of antibody that specifically binds to FcγRIIb administered between the first dose and the second dose is from around 30 mg to around 3000 mg. In some embodiments, the total dose of antibody that specifically binds to FcγRIIb administered between the first dose and the second dose is from around 0.3 mg/kg to around 20 mg/kg. In some other embodiments, the total dose between the first and second antibody doses is a dose that results in high receptor saturation, at least transiently, of the FcγRIIb receptor, for example at least 90% receptor saturation. In some preferred embodiments, this high receptor saturation persists for a total duration ranging from about 1 hour to about 4 weeks.

The skilled person will appreciate that the second dose of the antibody molecule that binds specifically to FcγRIIb may be adjusted based on the amount of the first dose of the antibody molecule that is administered.

In some preferred embodiments, the second dose of the antibody molecule that specifically binds to the FcγRIIb receptor is administered at a dose of from about 0.1 mg/kg to about 19.8 mg/kg.

It will be appreciated that, depending on the weight of the subject to whom the antibody molecule is to be administered, the second dose of the antibody molecule that specifically binds to FcγRIIb may be administered at a dose of from about 20 mg to about 2900 mg.

In preferred embodiments of the invention, further additional doses of the antibody molecule that specifically binds to FcγRIIb are administered to the subject following the second dose of the antibody molecule that specifically binds to FcγRIIb.

In preferred embodiments, the further additional doses of the antibody molecule that specifically binds to FcγRIIb are also administered according to the dosage regimens disclosed herein. For instance, this dosage regimen can be employed each and every time the antibody molecule that specifically binds to FcγRIIb is administered to the patient. In some embodiments, the exact format of the dosage regimen (in terms of timing and amounts of doses) may be varied between repeat administrations to the patient. The advantage of using the dosage regimens described herein repeatedly is that it ensures that the improved tolerability (for example a reduction in infusion related reactions) is achieved with each administration of the antibody that specifically binds FcγRIIb. In some other embodiments, the further additional doses of the antibody molecule are administered at the maximum therapeutically effective dose as defined herein. Typically, repeat doses will be similar in magnitude to the previously administered dose—for example:
if the previous administered antibody dose was 1.3 mg/kg (e.g. 0.3 mg/kg (first dose)+1 mg/kg (second dose)), the subsequent additional dose would also be 1.3 mg/kg;
if the previous administered antibody dose was 2.5 mg/kg (e.g. 0.5 mg/kg (first dose)+2 mg/kg (second dose)), the subsequent additional dose would also be 2.5 mg/kg;
if the previous administered antibody dose was 3.3 mg/kg (0.3 mg/kg (first dose)+3 mg/kg (second dose)), the subsequent additional dose would also be 3.3 mg/kg;
if the previous administered antibody dose was 5.4 mg/kg (e.g. 0.4 mg/kg (first dose)+5 mg/kg (second dose)), the subsequent additional dose would also be 5.4 mg/kg;
if the previous administered antibody dose” was 10.5 mg/kg (e.g. 0.5 mg/kg (first dose)+10 mg/kg (second dose)), the subsequent additional dose would also be 10.5 mg/kg.

However, as a person skilled in the art will appreciate, repeat dosing could also utilise higher or lower total doses as guided by patient tolerability. Analogous flat dosing-based, or receptor-occupancy guided, dosing regimens regimens could be used.

It will be appreciated that an antibody molecule that specifically binds to FcγRIIb has particular utility when administered with certain therapeutic antibodies, and particularly therapeutic antibodies used in the treatment of cancer or an inflammatory disease.

It is well known that many therapeutic antibodies exert their therapeutic effects by stimulating the removal of cancer and other unwanted cells by recruiting natural effector systems such as cytotoxic cells (e.g. macrophages) and enzymes (e.g. complement) which then target the cell to which the therapeutic antibody is bound. For example, Type I anti-CD20 monoclonal antibodies (such as the current market leader rituximab) work by binding to CD20 molecules on the surface of B cells, and deleting these target B cells. They do this through recruiting and activating effector cells which interact with the Fc domains of the therapeutic antibody through Fcγ (i.e. Fc-gamma) receptors expressed on the surface of these effector cells.

It is known that a factor determining the effectiveness of such therapeutic antibodies (such as those to antigens such as CD20) is interaction with the inhibitory FcγRIIb (also known as and including, CD32, CD32B, CD32B1, CD32B2, FcRII, FcγRII or FcRIIB). FcgRIIB may reduce therapeutic effectiveness and promote resistance to cancer by several mechanisms acting in cis i.e. on a cell targeted by a therapeutic antibody, or trans i.e. on a neighbouring effector cell which engages through its Fc gamma receptors in binding to the constant domain of antibodies coated on the surface of the antibody targeted cell. For example, this interaction may lead to internalisation of the therapeutic antibody by the target cell, thus removing its ability to interact with effector cell Fc receptors. It is known that agents which bind to FcγRIIb on the target cell (such as an antibody molecule that specifically binds to FcγRIIb) block this internalisation, and improve the activity of the therapeutic antibody.

Accordingly, in particularly preferred embodiments, the invention provides a system, combination, use, or method which further comprises administration of one or more therapeutic antibodies for the treatment of cancer or an inflammatory disease in a subject.

Preferably, the one or more therapeutic antibody is selected from the group consisting of:
one or more anti-PD1 antibody (such as pembrolizumab, nivolumab, cemiplimab, camrelizumab, dostarlimab, and/or biosimilars thereof);
one or more anti-CD20 antibody (such as rituximab, obinutuzumab, ofatumumab, and/or biosimilars thereof; for example as discussed in Roghanian et al., Cancer Cell, 2015, 27:473-488);
one or more anti-CD19 antibody (such as Loncastuximab tesirine);
one or more anti-CD40 antibody (such as CP-870,893);
one or more anti-CD38 antibody (for example, as described in Vaughan et al., Blood, 2014, 123:669-677 or daratumumab or a daratumumab biosimilar);
one or more anti-Her2 antibody (such as trastuzumab or a trastuzumab biosimilar);
one or more anti-EGFR antibody (such as cetuximab or a cetuximab biosimilar).

Preferably, the therapeutic antibody is one or more selected from the group comprising: rituximab; pembrolizumab; nivolumab; cemiplimab; camrelizumab; dostarlimab; obinutuzumab; ofatumumab, and biosimilars or equivalents thereof.

It will be appreciated that the doses and dosage regimens of each of the therapeutic antibodies discussed and contemplated herein would be dependent on the approved doses/regimens for these therapeutic antibodies, and would also vary depending on the indication (for example type of cancer/stage) and subject (for example BMI or age).

For example, in some embodiments wherein the therapeutic antibody is rituximab, the dose and dosage regimen may be as defined in the FDA label (see https://www.accessdata.fda.gov/drugsatfda_docs/label/2010/103705s5311lbl.pdf). As described therein, the doses may be as follows:
Non-Hodgkin's lymphoma (NHL): 375 mg/m2 once weekly for 4-8 doses;
Chronic Lymphocytic Leukemia (CLL): 375 mg/m2 the day prior to initiation of FC chemotherapy, then 500 mg/m2 of day 1 of cycles 2-6 (every 28 days);
Rheumatoid Arthritis (RA): administer as two 1000 mg insfusions separated by two weeks.

In another example, wherein the therapeutic antibody is pembrolizumab, the dose and dosage regimen may be as defined in the FDA label (see https://www.accessdata.fda.gov/drugsatfda_docs/label/2019/125514s040lbl.pdf). As described therein, the doses may be as follows:
Melanoma: 200 mg every 3 weeks;
Non-small cell lung cancer (NSCLC): 200 mg every 3 weeks;
Head and neck squamos cell carcinoma (HNSCC): 200 mg every 3 weeks;
Classical Hodgkin lymphoma (cHL) or Primary mediastinal large B-cell lymphoma (PMBCL): 200 mg every 3 weeks for adults; 2 mg/kg (up to 200 mg) every 3 weeks for pediatrics;
Urothelial Carcinoma: 200 mg every 3 weeks;
Microsatellite instability-high (MSI-H) Cancer: 200 mg every 3 weeks for adults and 2 mg/kg (up to 200 mg) every 3 weeks for pediatrics;
Gastric Cancer: 200 mg every 3 weeks;
Cervical Cancer: 200 mg every 3 weeks;
Hepatocellular carcinoma (HCC): 200 mg every 3 weeks;
Merkel cell carcinoma (MCC): 200 mg every 3 weeks for adults; 2 mg/kg (up to 200 mg) every 3 weeks for pediatrics.

The term “subject” (which herein is used interchangeably with “patient”) includes any animal, including a human, that is in need of treatment with an antibody molecule that specifically binds to FcγRIIb. The subject or patient may be mammalian or non-mammalian. Preferably, the subject is mammalian, such as a horse, or a cow, or a sheep, or a pig, or a camel, or a dog, or a cat. Most preferably, the mammalian patient is a human.

Preferably, the subject is one that has been diagnosed as having cancer or an inflammatory disease, or that has been identified as likely to have cancer or an inflammatory disease and/or that exhibits symptoms of cancer or an inflammatory disease.

In other preferred embodiments, the subject is one that has been diagnosed as having an infectious disease, or that has been identified as likely to have an infectious disease and/or that exhibits symptoms of an infectious disease. By infectious disease we include any disease caused by bacteria, fungi, parasites or viruses that can be transmitted between persons (either directly or indirectly).

In some other embodiments, the subject has cancer and/or an inflammatory disease and/or an infectious disease and the dosage regimens described herein are used in conjunction with administration of a vaccine aimed at boosting humoral or cellular responses in order to treat and/or prevent said cancer or disease.

By “exhibits”, we include that the subject displays a cancer symptom and/or a cancer diagnostic marker, and/or the cancer symptom and/or a cancer diagnostic marker can be measured, and/or assessed, and/or quantified. It would be readily apparent to the person skilled in medicine what the cancer symptoms and cancer diagnostic markers would be and how to measure and/or assess and/or quantify whether there is a reduction or increase in the severity of the cancer symptoms, or a reduction or increase in the cancer diagnostic markers; as well as how those cancer symptoms and/or cancer diagnostic markers could be used to form a prognosis for the cancer.

In some embodiments, the cancer is a FcγRIIb-positive cancer. In other embodiments, the cancer is an FcγRIIb-negative cancer.

By “FcγRIIb-positive cancer”, we include any cancer that expresses FcγRIIb, albeit at different levels. FcγRIIb expression is most pronounced in chronic lymphocytic leukaemia and mantle cell lymphomas, moderately so in diffuse large B cell lymphoma and least pronounced in follicular lymphomas. However, in some cases subjects with cancers that generally express low levels of FcγRIIB (e.g. follicular lymphomas) may have very high levels of FcγRIIb expression.

By “FcγRIIb negative cancer” we include any cancer that does not present any FcγRIIb receptors. This can be tested using anti-FcγRIIB specific antibodies in a variety of methods including immunohistochemistry and flow cytometry such as indicated in Tutt et al, J Immunol, 2015, 195 (11) 5503-5516.

In some preferred embodiments, the cancer is selected from the group consisting of carcinomas, sarcomas, and lymphomas. In some embodiments, the cancer is a carcinoma selected from the group consisting of adenocarcinoma, squamous cell carcinoma, adenosquamous carcinoma, anaplastic or undifferentiated carcinoma, large cell carcinoma and small cell carcinoma. In some embodiments, the cancer is a sarcoma selected from the group consisting of osteosarcoma, chondrosarcoma, liposarcoma, and leiomyosarcoma.

In some preferred embodiments, the cancer is selected from the group of cancers indicated in the label of an approved therapeutic antibody to be co-administered with the anti-FcgRIIB antibody. By co-administered we mean an antibody used as part of the anti-FcgRIIB antibody comprising therapy, where the co-administered antibody may be administered before, concomitantly, or subsequent in time to the anti-FcgRIIB antibody.

In some preferred embodiments, the disease is selected from the group of diseases indicated in the label of an approved therapeutic antibody co-administered with the anti-FcgRIIB antibody. By co-administered we mean an antibody used as part of the anti-FcgRIIB antibody comprising therapy, where the co-administered antibody may be administered before, concomitantly, or subsequent in time to the anti-FcgRIIB antibody.

The cancer may be selected from the group comprising: melanoma, breast cancer, ovarian cancer, cervical cancer, prostate cancer, metastatic hormone-refractory prostate cancer, colorectal cancer, lung cancer, small cell lung carcinoma, small cell lung cancer (SCLC), non-small cell lung cancer, urothelial carcinoma, bladder cancer, kidney cancer, mesothelioma, Merkel cell carcinoma, head and neck cancer, and pancreatic cancer.

Preferably, the cancer is a B-cell cancer, such as a cancer selected from the group comprising: chronic lymphocytic leukaemia, mantle cell lymphoma, follicular lymphoma, diffuse large B cell lymphoma.

Each one of the above described cancers is well-known, and the symptoms and cancer diagnostic markers are well described, as are the therapeutic agents used to treat those cancers. Accordingly, the symptoms, cancer diagnostic markers, and therapeutic agents used to treat the above mentioned cancer types would be known to those skilled in medicine.

Clinical definitions of the diagnosis, prognosis and progression of a large number of cancers rely on certain classifications known as staging. Those staging systems act to collate a number of different cancer diagnostic markers and cancer symptoms to provide a summary of the diagnosis, and/or prognosis, and/or progression of the cancer. It would be known to the person skilled in oncology how to assess the diagnosis, and/or prognosis, and/or progression of the cancer using a staging system, and which cancer diagnostic markers and cancer symptoms should be used to do so.

By “cancer staging”, we include the Rai staging, which includes stage 0, stage I, stage II, stage III and stage IV, and/or the Binet staging, which includes stage A, stage B and stage C, and/or the Ann Arbour staging, which includes stage I, stage II, stage III and stage IV.

It is known that cancer can cause abnormalities in the morphology of cells. These abnormalities often reproducibly occur in certain cancers, which means that examining these changes in morphology (otherwise known as histological examination) can be used in the diagnosis or prognosis of cancer. Techniques for visualizing samples to examine the morphology of cells, and preparing samples for visualization, are well known in the art; for example, light microscopy or confocal microscopy.

By “histological examination”, we include the presence of small, mature lymphocyte, and/or the presence of small, mature lymphocytes with a narrow border of cytoplasm, the presence of small, mature lymphocytes with a dense nucleus lacking discernible nucleoli, and/or the presence of small, mature lymphocytes with a narrow border of cytoplasm, and with a dense nucleus lacking discernible nucleoli, and/or the presence of atypical cells, and/or cleaved cells, and/or prolymphocytes.

It is well known that cancer is a result of mutations in the DNA of the cell, which can lead to the cell avoiding cell death or uncontrollably proliferating. Therefore, examining these mutations (also known as cytogenetic examination) can be a useful tool for assessing the diagnosis and/or prognosis of a cancer. An example of this is the deletion of the chromosomal location 13q14.1 which is characteristic of chronic lymphocytic leukaemia. Techniques for examining mutations in cells are well known in the art; for example, fluorescence in situ hybridization (FISH).

By “cytogenetic examination”, we include the examination of the DNA in a cell, and, in particular the chromosomes. Cytogenetic examination can be used to identify changes in DNA which may be associated with the presence of a refractory cancer and/or relapsed cancer. Such may include: deletions in the long arm of chromosome 13, and/or the deletion of chromosomal location 13q14.1, and/or trisomy of chromosome 12, and/or deletions in the long arm of chromosome 12, and/or deletions in the long arm of chromosome 11, and/or the deletion of 11q, and/or deletions in the long arm of chromosome 6, and/or the deletion of 6q, and/or deletions in the short arm of chromosome 17, and/or the deletion of 17p, and/or the t(11:14) translocation, and/or the (q13:q32) translocation, and/or antigen gene receptor rearrangements, and/or BCL2 rearrangements, and/or BCL6 rearrangements, and/or t(14:18) translocations, and/or t(11:14) translocations, and/or (q13:q32) translocations, and/or (3:v) translocations, and/or (8:14) translocations, and/or (8:v) translocations, and/or t(11:14) and (q13:q32) translocations.

It is known that patients with cancer exhibit certain physical symptoms, which are often as a result of the burden of the cancer on the body. Those symptoms often reoccur in the same cancer, and so can be characteristic of the diagnosis, and/or prognosis, and/or progression of the disease. A person skilled in medicine would understand which physical symptoms are associated with which cancers, and how assessing those physical systems can correlate to the diagnosis, and/or prognosis, and/or progression of the disease. By “physical symptoms”, we include hepatomegaly, and/or splenomegaly.

In some embodiments, the cancer is one that is resistant to treatment with a therapeutic anti-cancer antibody. Such resistant cancer may be a relapsed and/or refractory cancer.

A relapsed cancer is a cancer that has previously been treated and, as a result of that treatment, the subject made a complete or partial recovery (i.e. the subject is said to be in remission), but that after the cessation of the treatment the cancer returned or worsened. Put another way, a relapsed cancer is one that has become resistant to a treatment, after a period in which it was effective and the subject made a complete or partial recovery.

A refractory cancer is a cancer that has been treated but which has not responded to that treatment, and/or has been treated but which has progressed during treatment. Put another way, a refractory cancer is one that is resistant to a treatment. It will be appreciated that a cancer may be a refractory cancer due to an intrinsic resistance. By “intrinsic resistance”, we include the meaning that the cancer and/or the subject and/or the target cell is resistant to a particular treatment from the first time at which it is administered, or before it is administered at all.

A relapsed cancer and/or refractory cancer would be readily diagnosed by one skilled in the art of medicine.

In embodiments of the invention, the antibody molecule that specifically binds to FcγRIIb is formulated and/or adapted for delivery by a route selected from the group comprising: intravenous; intramuscular; subcutaneous. In some embodiments, the antibody molecule that specifically binds to FcγRIIb is formulated and/or adapted for intravenous (i.e. i.v. or iv) delivery. In other embodiments, the antibody molecule that specifically binds to FcγRIIb is formulated and/or adapted for subcutaneous (i.e. s.c. or sc) delivery.

In embodiments of the invention, the antibody molecule that specifically binds to FcγRIIb is delivered to the subject by a route selected from the group comprising: intravenous;

intramuscular; subcutaneous. Preferably, the antibody molecule that specifically binds to FcγRIIb is delivered intravenously.

Thus, in preferred embodiments, the first and/or second and/or further doses of the antibody molecule that specifically binds to FcγRIIb are formulated for intravenous delivery to the subject and/or are delivered by intravenous delivery to the subject.

Methods and formulations for intravenous administration of antibody molecules are well known in the art. In the present invention, any type of intravenous administration may be used, such as injection or infusion.

In embodiments of the invention, the corticosteroid is formulated and/or adapted for delivery by a route selected from the group comprising: intravenous; oral.

In embodiments of the invention, the corticosteroid is delivered to the subject by a route selected from the group comprising: intravenous; oral.

Thus, in preferred embodiments, the first and/or second and/or further doses of the corticosteroid are formulated for intravenous or oral delivery to the subject and/or are delivered by intravenous or oral delivery to the subject.

Methods and formulations for intravenous or oral administration of corticosteroids are well known in the art.

The antibody molecule that specifically binds FcγRIIb and/or the corticosteroid as defined herein may be combined with an excipient and/or a pharmaceutically acceptable carrier and/or a pharmaceutically acceptable diluent and/or an adjuvant.

For example, the antibody that specifically binds FcγRIIb and/or the corticosteroid may be formulated as an aqueous and/or non-aqueous sterile solution which may contain anti-oxidants, and/or buffers, and/or bacteriostats, and/or solutes which render the formulation isotonic with the blood of the intended recipient; and/or aqueous and/or non-aqueous sterile suspensions which may include suspending agents and/or thickening agents. Such formulations may be presented in unit-dose or multi-dose containers, for example sealed ampoules and vials, and may be stored in a freeze-dried (i.e. lyophilised) condition requiring only the addition of the sterile liquid carrier, for example water, immediately prior to use.

Extemporaneous injection solutions and suspensions may be prepared from sterile powders, and/or granules, and/or tablets of the kind known in the art.

The antibody that specifically binds FcγRIIb and/or the corticosteroid may be formulated with pharmaceutically acceptable acid or base addition salts. The acids which are used to prepare the pharmaceutically acceptable acid addition salts are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others. Pharmaceutically acceptable base addition salts may also be used to produce pharmaceutically acceptable salt forms. The chemical bases that may be used as reagents to prepare pharmaceutically acceptable base salts are those that form non-toxic base salts. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The antibody molecule that specifically binds FcγRIIb and/or corticosteroid may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) antibody molecule loses no more than about 20%, or no more than about 25%, or no more than about 30%, or no more than about 35%, or no more than about 40%, or no more than about 45%, or no more than about 50% of its activity (prior to lyophilisation) when re-hydrated.

As discussed above and as demonstrated in the accompanying Examples, the system, combination, method or use of the present invention improves tolerability of an antibody molecule that specifically binds to FcγRIIb in a subject. Put another way, the system, combination, method or use of the present invention reduces or prevents adverse effects associated with administration of the antibody molecule (and, particularly, with the intravenous administration of the antibody molecule).

In a preferred embodiment of the system, combination, method or use of the present invention, the infusion related reactions associated with the administration of the antibody molecule that specifically binds to FcγRIIb are reduced or eliminated. Such infusion related reactions are described herein, and it is contemplated that the system, combination, method or use of the present invention reduces or eliminates any one or more of those infusion related reactions.

In a preferred embodiment, changes to the body temperature and/or platelet count and/or blood levels of liver enzymes (e.g. ALAT or ASAT) and/or blood levels of cytokines (e.g. IL-6) of the subject are reduced. Preferably IRRs are reduced to acceptable levels, i.e. below grade 3 as defined by Common Terminology for Adrerse Events (CTCAE) as defined herein for at least 24 hours following administration of the second dose of the antibody molecule that specifically binds to FcγRIIb. Most preferably IRRs are completely prevented with normalized body temperature and/or platelet count and/or blood levels of liver enzymes (e.g. ALAT or ASAT) and/or blood levels of cytokines (e.g. IL-6) of the subject.

As defined above, the normal levels of some of these parameters are as follows:
Body temperature: from 36.1° C. to 37.9° C.;
Platelet count: from 145×10⁹ to 400×10⁹ per litre;
Blood level of ALAT: from 0 to 1.09 μkat/L, 16-63 U/L;
Blood level of ASAT: from 0 to 0.759 μkat/L, 15-37 U/L;
Blood level IL-6: from 0.16 to 27.2 pg/ml, with a median value of 0.47 pg/ml.

By “acceptable levels” we include that the clinical grading of the IRR (as defined in the art and herein using the CTCAE scale) is reduced to at least grade 2. In some preferred embodiments, the grading of the IRR is reduced to grade 1. As discussed herein, the skilled person will be aware of how to grade an IRR according to the CTCAE scale.

A fifth aspect of the invention provides a kit comprising:
(i) an antibody molecule that specifically binds to FcγRIIb, preferably an antibody molecule as defined herein;
(ii) a corticosteroid, preferably a corticosteroid as defined herein; and
(iii) optionally, instructions for use,
   wherein the antibody molecule is provided as at least a first dose and a second dose, wherein the first dose of the antibody molecule is lower than the maximum therapeutically effective dose of the antibody molecule, further optionally wherein the first dose is as defined herein, further optionally wherein the second dose is as defined herein. The antibody molecule and doses of that aspect of the invention are as defined herein.

Preferably, the kit of the invention is for improving the tolerability of the antibody molecule in a subject, as is described herein.

Preferably, in the kit of the invention, the corticosteroid is provided in a dose as defined herein.

In a preferred embodiment, the kit of the invention further comprises one or more therapeutic antibodies, as defined herein. For example, the therapeutic antibody is one or more selected from the group comprising: rituximab; pembrolizumab; nivolumab;

cemiplimab; camrelizumab; dostarlimab; obinutuzumab; ofatumumab, and biosimilars or equivalents thereof.

It will be appreciated that, where one or more therapeutic antibody is present in the kit, the kit of the invention is for use in treating cancer in a subject, as is described herein.

### Further Aspects of the Invention

In a sixth aspect, disclosed herein is a method (or model) for predicting if a therapeutic antibody molecule binding specifically to a human target will be associated with a tolerability issue in connection with intravenous administration to a human, comprising the following step:
(i) intravenous or intraperitoneal administration of the therapeutic antibody molecule, if cross-reactive with murine target, or a surrogate antibody, to a mouse and observation of the mouse during a period following immediately after the administration of the therapeutic or surrogate antibody, wherein a display of the macroscopic symptoms isolation and decreased activity during the period followed by restoration of the state of the mouse to the normal state is an indication that the intravenous administration of the therapeutic antibody molecule to a human will be associated with a tolerability issue,
   and/or for predicting if a prophylactic or therapeutic treatment, an altered administration route and/or a modification of the therapeutic antibody molecule can prevent or mitigate a tolerability issue associated with intravenous administration to a human of a therapeutic antibody molecule binding specifically to a human target,
   comprising the following step(s) in addition to (i) as set out above:
(ii) administration of a prophylactic or therapeutic agent to a mouse in conjunction with intravenous or intraperitoneal administration of the therapeutic or surrogate antibody to a mouse, and observation of the mouse during a period following immediately after the administration of the therapeutic or surrogate antibody, wherein a decreased display of the macroscopic symptoms compared to the macroscopic symptoms displayed by the mouse in (i) or no display of the macroscopic symptoms during the period is an indication that pre-treatment with the prophylactic or therapeutic agent in combination with administration of the therapeutic antibody molecule to a human can prevent or mitigate the tolerability issue that otherwise would be associated with intravenous administration of the therapeutic antibody molecule to a human;
(iii) administration of the therapeutic or surrogate antibody to a mouse by a route of administration other than intravenous or intraperitoneal administration, and observation of the mouse during a period following immediately after the administration of the therapeutic or surrogate antibody, wherein a decreased display of the macroscopic symptoms compared to the macroscopic symptoms displayed by the mouse in (i) or no display of the macroscopic symptoms during the period is an indication that the other route of administration can be used for administration of the therapeutic antibody molecule to a human to prevent or mitigate the tolerability issue that would be associated with intravenous administration of the therapeutic antibody molecule to a human; and/or
(iv) intravenous or intraperitoneal administration of a modified format of the therapeutic or surrogate antibody to a mouse by a route of administration other than intravenous or intraperitoneal administration, and observation of the mouse during a period following immediately after the administration of the modified therapeutic or surrogate antibody, wherein a decreased display of the macroscopic symptoms compared to the macroscopic symptoms displayed by the mouse in (i) or no display of the macroscopic symptoms during the period is an indication that administration of the therapeutic antibody molecule in the modified format to a human can be used to prevent or mitigate the tolerability issue that would be associated with intravenous administration of the therapeutic antibody molecule to a human.

In a seventh aspect, disclosed herein is also a corticosteroid for use in a dosing regimen to prevent or mitigate a tolerability issue in connection with intravenous administration of a therapeutic antibody molecule to a subject,

wherein the therapeutic antibody molecule has been predicted to be associated with a tolerability issue in connection with intravenous administration to a human using the method described above, and/or wherein pre-treatment with the corticosteroid combination with administration of the therapeutic antibody molecule to a human has been predicted to prevent or mitigate the tolerability issue that otherwise would be associated with intravenous administration of the therapeutic antibody molecule to a human using the method described above,
and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).

A variant of this aspect relates to a corticosteroid for use in a dosing regimen to prevent or mitigate a tolerability issue in connection with intravenous administration of a therapeutic antibody molecule to a subject,

wherein the therapeutic antibody molecule is an anti-FcγRIIB antibody,
and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).

In an eighth aspect, disclosed herein is also a therapeutic antibody molecule for use in the treatment of cancer, wherein the therapeutic antibody molecule has been predicted to be associated with a tolerability issue in connection with intravenous administration to a human using the method described above and/or wherein the subcutaneous route of administration of therapeutic antibody molecule to a human has been predicted to prevent or mitigate the tolerability issue that otherwise would be associated with intravenous administration of the therapeutic antibody molecule to a human using the method described above, and wherein the antibody is formulated for subcutaneous administration.

In a ninth aspect, disclosed herein is also a modified format of a therapeutic antibody molecule for use in the treatment of cancer, wherein the therapeutic antibody molecule has been predicted to be associated with a tolerability issue in connection with intravenous administration to a human using the method described above and/or wherein administration of the therapeutic antibody molecule in the modified format to a human has been predicted to prevent or mitigate the tolerability issue that otherwise would be associated with intravenous administration of the therapeutic antibody molecule to a human using the method described above, and wherein the therapeutic antibody molecule is an Fc receptor binding antibody and the modified format is an antibody having the same Fv variable sequence but having reduced, impaired or abrogated FcγR binding compared with the therapeutic antibody molecule.

In a tenth aspect, disclosed herein is also a method for preventing or mitigating a tolerability issue in connection with intravenous administration of a therapeutic antibody molecule to a subject comprising a corticosteroid dosing regimen, wherein the therapeutic antibody molecule has been predicted to be associated with a tolerability issue in connection with intravenous administration to a human using the predictive method described above, and/or wherein pre-treatment with the corticosteroid combination with administration of the therapeutic antibody molecule to a human has been predicted to prevent or mitigate the tolerability issue that otherwise would be associated with intravenous administration of the therapeutic antibody molecule to a human using the predictive method described above, and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).

In an eleventh aspect, disclosed is also a method for treatment of cancer comprising subcutaneous administration of a therapeutically active amount of a therapeutic antibody molecule which has been predicted to be associated with a tolerability issue in connection with intravenous administration to a human using the predictive method described above and/or wherein the subcutaneous route of administration of therapeutic antibody molecule to a human has been predicted to prevent or mitigate the tolerability issue that otherwise would be associated with intravenous administration of the therapeutic antibody molecule to a human using the predictive method described above.

In a twelfth aspect, disclosed is also a method for treatment of cancer comprising administration of a therapeutically active amount of a modified format of a therapeutic antibody, wherein the therapeutic antibody molecule has been predicted to be associated with a tolerability issue in connection with intravenous administration to a human using the predictive method described above and/or wherein administration of the therapeutic antibody molecule in the modified format to a human has been predicted to prevent or mitigate the tolerability issue that otherwise would be associated with intravenous administration of the therapeutic antibody molecule to a human using the predictive method described above, and wherein the therapeutic antibody molecule is an Fc receptor binding antibody and the modified format is an antibody having the same Fv variable sequence but having impaired or abrogated FcγR binding compared with the therapeutic antibody molecule.

### Detailed Description of the Further Aspects of the Invention

In brief, in these further aspects of the invention, we describe a model for predicting if a therapeutic antibody binding to a human target will be associated with a tolerability issue in connection with intravenous administration and/or for predicting if pre-treatment, altered administration route or modification of the antibody can prevent a tolerability issue associated with intravenous administration to a human of the therapeutic antibody. The model comprises administering the antibody intravenously or intraperitoneally to mice and observing the mice immediately after the administration for any transient display of the macroscopic symptoms isolation and decreased activity. The model may also comprise administration of a pre-treatment in combination with administration of the antibody, administration of the therapeutic antibody by a route of administration other than intravenous or intraperitoneal administration or administration of a modified format of the antibody to mice and observing the mice immediately after such administration for any transient display of the macroscopic symptoms isolation and decreased activity and comparing this with the transient display of the macroscopic symptoms isolation and decreased activity after the intravenous or intraperitoneal administration of the unmodified antibody without pre-treatment. Analyses of associated microscopic symptoms, changes in biochemical, or cellular parameters may help gathering information of the nature of the IRR, and guide candidate preventative pre-medication or IRR reducing interventions to test in the model, as described below.

The predictive method described herein in the further aspects of the invention makes it possible to predict if a therapeutic antibody molecule to a given target will be associated with—or is likely to be associated with—a tolerability issue in connection with its intravenous administration to a human subject. Additionally or alternatively, it makes it possible to predict if a prophylactic or therapeutic treatment, an altered administration route and/or a modification of the therapeutic antibody molecule can be used to prevent or mitigate a tolerability issue associated with intravenous administration to a human of a therapeutic antibody molecule binding specifically to a human target.

In these further aspects of the invention, the therapeutic antibody molecule binds specifically to a human target. That the antibody binds specifically to the target means that it specifically binds to or interacts with a defined target molecule or antigen, and that this means that the antibody preferentially and selectively binds its target and not a molecule which is not a target.

The target to which the therapeutic antibody molecule binds may be a receptor or antigen found on any human cell. Examples of such cells are leukocytes, myeloid cells and B cells.

In some embodiments the target is FcγRII (CD32).

In some embodiments the target is FcγRIIB (CD32b).

In some embodiments the target is FcγRIIA (CD32a).

In some embodiments the target is CD40.

In these further aspects of the invention, the therapeutic antibody molecule may be any therapeutic antibody molecule that has received regulatory approval for use in humans or a therapeutic antibody molecule in clinical development or may be any antibody binding to a human target antigen intended or hypothesized to be of use for therapy of human disease. The term therapeutic antibody molecule as used herein also encompasses antibodies that are envisaged to be considered for, or are being developed for, therapeutic use, including antibodies in preclinical development. The therapeutic antibody molecule is thus an antibody that has therapeutic effects on humans. In the predictive method described herein, the therapeutic antibody molecule (if cross-reactive), or a surrogate antibody to the analogous mouse target, may be administered to a mouse. The mouse used is an immune competent laboratory mouse. Mice of different genetic background, inbred or outbred, may be used. Further, mice transgenic for the human target of the therapeutic antibody molecule may be used.

Often, such a therapeutic antibody molecule is a monoclonal antibody. In many cases it is a human or humanized antibody.

In these further aspects of the invention, the therapeutic antibody molecule may be any type of antibody, such as immunoglobulin G (IgG), immunoglobulin A (IgA) or immunoglobulin M (IgM). In some embodiments, it is IgG. It may also be of any subclass, such as IgG1, IgG2, IgG3 or IgG4. It may also be an antibody molecule engineered for enhanced, reduced, or diminished FcγR-dependent engagement and function. Further, the therapeutic antibody molecule may be a mono-, bi- or tri-specific for the same or different targets and comprising or being fused to an antibody Fc domain. Moreover, the therapeutic antibody molecule may be a mono-, bi- or tri-specific for the same or different targets and not comprising an antibody Fc domain. Further, the therapeutic antibody molecule may be a functional fragment of an antibody, such as an Fv, consisting of the variable domain of an antibody, a Fab, also denoted F(ab), which is a monovalent antigen-binding fragment that does not contain a Fc part, or a F(ab′)₂, which is an divalent antigen-binding fragment that contains two antigen-binding Fab parts linked together by disulfide bonds, or a F(ab′), i.e. a monovalent-variant of a F(ab′)₂. Such a fragment may also be single chain variable fragment (scFv).

In some embodiments, the therapeutic antibody molecule is an antibody used in or intended for cancer therapy. Monoclonal antibodies have been and are being developed for a number of cancers, and more will likely follow. Some examples are brain cancer, breast cancer, chronic lymphocytic leukemia, colorectal cancer, head and neck cancers, Hodgkin's lymphoma, lung cancer, melanoma, non-Hodgkin's lymphoma, prostate cancer and stomach cancer. Some antibodies are approved for use in different indications. For example the anti-CD20 antibody rituximab is approved for use in both cancer (NHL and CLL) and autoimmune disease (rheumatoid arthritis). The method described herein is however not limited to antibodies used in cancer therapy or therapy of autoimmune/inflammatory disease.

As mentioned above, in some embodiments the target is FcγRIIB. In some preferred embodiments, the therapeutic antibody molecule has a light chain with SEQ ID No:1 and a heavy chain with SEQ ID No:2.

In these further aspects of the invention, the intravenous (iv) administration method used to administer the therapeutic antibody molecule to a human may be any type of intravenous administration, such as by injection or by infusion.

The predictive method described herein in these further aspects of the invention utilizes mice as a model to predict what will happen in humans. When the therapeutic antibody molecule to be tested is cross reactive with a known murine homologue of the human target, the therapeutic antibody molecule may be used in the predictive method. When the therapeutic antibody molecule to be tested is not cross reactive with a known murine homologue of the human target, it is necessary to use a surrogate antibody. The surrogate antibody is an antibody that is specific for a murine homologue of the human target to which the therapeutic antibody molecule binds. For example, in this context the murine homologue of the human target FcγRIIB is murine FcγRII, the murine homologue of the human target FcγRIIA is murine FcγRIII, and the murine homologue of the human target CD40 is murine CD40. The surrogate antibody may be a murine antibody or an antibody from another species, such as rat, rabbit, monkey or chicken. Sometimes, it may be preferable to use a surrogate antibody even if the therapeutic antibody molecule to be tested is cross reactive with a known murine homologue of the human target. This may be the case if the surrogate antibody's binding and interaction with mouse target antigen and mouse immune proteins regulating antibody activity e.g. FcγRs better reflect the interactions of the human candidate antibodies interaction with human target and human immune proteins regulating antibody activity e.g. FcγRs compared with the therapeutic antibody molecule itself. Preferably, and when available, both cross-reactive therapeutic antibody molecule and murine surrogate antibodies, may be used in parallel to test for tolerability issues as described herein.

In these further aspects of the invention, the therapeutic antibody molecule may be an antibody that binds or does not bind Fc receptors. A modified format can then be used, i.e. antibody variants with lower Fc-FcgR-engagement owing to isotype switching or Fc-engineering. If a surrogate antibody is used to predict or model tolerability issues of a therapeutic antibody molecule to the same or homologous target, then the Fc of the surrogate antibody should be chosen to match the therapeutic antibody molecule's Fc with respect to binding/non-binding (or engagement/non-engagement) of FcγR-binding and function. It is, for example, well known that both human IgG1, IgG3 and IgG4 productively bind and engage human FcγRs albeit with different absolute and relative affinities. Similarly, in the mouse, mIgG2a binds strongly and broadly to different mouse FcγRs, while mIgG1 binds only to mouse FcγRII and FcγRIII. It is further well known aglycosylation of antibodies, specifically in the 297 position (such as one of the following mutations: N297A, N297Q or N297G), render both human and mouse IgG impaired and/or reduced, or severely reduced, for binding to FcγR. In this context, Fc binding means that the Fc part of the therapeutic antibody molecule binds to an FcγR which leads to engagement of Fc:FcγR dependent activities or functions. Furthermore, impaired or abrogated FcγR binding means that the modified format does not bind at all to FcγR or that it binds less strongly to FcγR than the therapeutic, unmodified antibody.

In these further aspects of the invention, the therapeutic antibody molecule or the surrogate antibody is administered intravenously or intraperitoneally to a mouse. In some cases, the dose of the therapeutic antibody molecule or the surrogate antibody administered to the mouse is the dose that results in high receptor saturation. In some cases, the dose of the therapeutic antibody molecule or the surrogate antibody administered to the mouse is the dose that results in at least 90% receptor saturation. In some cases, the dose of the therapeutic antibody molecule or the surrogate antibody administered to the mouse is the dose that results in close to 100% or 100% receptor saturation.

Once the antibody has been administered to the mouse the animal is observed for visual physical reactions, in particular behavior changes or macroscopic symptoms, are observed. If the therapeutic antibody molecule is an antibody that will be associated with a tolerability issue in connection with intravenous administration to a human, the mouse will starting to show the macroscopic symptoms isolation and decreased activity very shortly, i.e. within a few minutes, such as 5-10 minutes, after administration of the therapeutic or surrogate antibody. In some cases, the mouse will also display signs of impaired balance, piloerection, and/or hunching followed by un-natural body posture. These three additional macroscopic symptoms will be observed within the same time frame as the isolation and decreased activity, i.e. within a few minutes, such as 5-10 minutes, after administration of the therapeutic or surrogate antibody. The display of one, two or three of these additional macroscopic symptoms is a stronger predictive marker that the therapeutic antibody molecule binding specifically to a human target is likely to be associated with a tolerability issue in connection with intravenous administration to a human compared to if the mouse would only show signs of isolation and decreased activity.

A person experienced in working with laboratory mice will immediately notice if the above changes occurs in the mouse's behavior, since the signs are easy to observe and is a notable change of the mouse behavior prior to administration of the therapeutic or surrogate antibody. The symptoms are clearly manifested, and it is obvious that the mouse is not feeling well. After a period ending in about one hour, such as 45 minutes to 1.5 hours, after injection of the antibody (therapeutic or surrogate), the mouse no longer shows any macroscopic symptoms. Instead, its behavior is restored to the normal state, i.e. to the behavior prior to administration of the antibody (therapeutic or surrogate).

In addition to the above macroscopic symptoms, the mouse may demonstrate other symptoms. One such symptom is decreased blood pressure. Another such symptom is decreased platelet count. Another such symptom is elevated levels of the two liver enzymes aspartate aminotransferase (AST) and alanine aminotransferase (ALT). Contrary to the macroscopic symptoms, these cannot be determined by simply observing the mouse. Instead they may be determined through blood analysis. To check these “non-macroscopic” symptoms, blood is drawn from the mouse approximately five minutes after injection of the antibody (therapeutic or surrogate). The blood is then analyzed for platelet count and/or levels of AST and/or ALT. Decreased blood pressure may also be determined this way; if the blood pressure is decreased, it will not be possible to draw blood from the mouse during the period during which the macroscopic symptoms are displayed. To determine if the platelet count is decreased and/or if the level of AST and/or ALT is elevated, it is possible to compare either with a blood sample drawn from the mouse prior to administration of the therapeutic antibody molecule or surrogate antibody or with a sample drawn from a control mouse. The decreased blood pressure will be restored within the same period as the macroscopic symptoms. The platelet count, AST level and ALT level will take a bit longer to be restored; it is normalized within 6-10 hours, such as within 8 hours.

The predictive method described herein in these further aspects of the invention can be used to predict if a therapeutic antibody molecule binding specifically to a human target will be associated with a tolerability issue in connection with intravenous administration to a human.

Moreover, the predictive method described herein in these further aspects of the invention can be used to test strategies for overcoming such a tolerability issue. More precisely, it can be used to predict if a specific strategy can prevent or mitigate a tolerability issue associated with intravenous administration to a human of a therapeutic antibody molecule binding specifically to a human target. This is useful for example for a therapeutic antibody molecule that is being clinically developed or is already used in the clinic for which tolerability issues have been observed.

Furthermore, the predictive method described herein In these further aspects of the invention can be used both to predict if a therapeutic antibody molecule binding specifically to a human target will be associated with a tolerability issue in connection with intravenous administration to a human and to predict if a specific strategy can prevent or mitigate the tolerability issue. This may be of interest for example when developing a drug since it enables both identification of a potential problems and means of finding a solution to the problem.

If the predictive method described herein in these further aspects of the invention is to be used only to predict if a therapeutic antibody molecule binding specifically to a human target will be associated with a tolerability issue in connection with intravenous administration to a human, the method is performed as described above, with administration of the therapeutic or surrogate antibody followed by observance of the mouse. Normally, not only one mouse would be used, but instead a test group of several mice, such as 5-10, would be used and the experiment would be repeated to ascertain that any change observed is representative, reproducible and statistically significant.

If the predictive method described herein in these further aspects of the invention is to be used only to, or in addition to, predict if a specific strategy can prevent or mitigate a tolerability issue associated with intravenous administration to a human of a therapeutic antibody molecule binding specifically to a human target, the method described above is performed on a control mouse, or preferably on a control group of mice, such as 5-10 mice. In addition, a second mouse, or preferably a second group of mice, such as 5-10 mice, is treated in accordance with the specific strategy that is to be tested. The results for the second mouse, or second group of mice, are compared to the results for the control mouse, or control group of mice.

Examples of strategies for overcoming tolerability issue arising in connection with intravenous administration of a therapeutic antibody molecule to a human are different prophylactic treatments, different therapeutic treatments, altered administration routes and/or modifications of the therapeutic antibody molecule. By testing such strategies as described herein, data will be obtained that can be used to predict if that particular strategy can be used to prevent or mitigate a tolerability issue that would be associated with intravenous administration of a specific therapeutic antibody molecule to a human. Thus, it is possible to obtain reliable data without having to test the effect of different strategies on humans that first have to experience the tolerability issue(s).

When the strategy for overcoming a tolerability issue is a prophylactic treatment, a prophylactic agent is administered to the second mouse, or second group of mice, prior to the intravenous or intraperitoneal administration of the therapeutic or surrogate antibody to the mouse, or group of mice. This strategy is thus pre-treatment with the prophylactic agent. The second mouse, or second group of mice, is observed during a period following immediately after the administration of the therapeutic or surrogate antibody The results for the second mouse, or second group of mice, are compared to the results for the control mouse, or control group of mice, which has not received the prophylactic agent. A decreased display of the macroscopic symptoms for the second mouse, or the second group of mice, compared to the control mouse, or control group of mice, or no display of the macroscopic symptoms at all for the second mouse, or second group of mice, during the period is an indication that administration of the prophylactic agent can be used to prevent or mitigate the tolerability issue that would be associated with intravenous administration of the therapeutic antibody molecule to a human.

The prophylactic agent tested this way may be any agent that is known to prevent or mitigate tolerability issues, or is hypothesized, or screened, for its ability to help mitigate tolerability issues.

In some embodiments of these further aspects of the invention, the prophylactic treatment is pre-treatment with a corticosteroid. In some such embodiments, the pre-treatment comprises two administrations of a corticosteroid. The corticosteroid is preferably a potent corticosteroid, and more preferably a corticosteroid with as high potency as possible or as available. Examples of such corticosteroids are dexamethasone and betamethasone.

When a pre-treatment with a corticosteroid, such as dexamethasone or betamethasone, is used it may comprise two administrations of the corticosteroid prior to administration of the therapeutic or surrogate antibody. In some such embodiments, one dose of corticosteroid is administered 10-48 hours prior to administration of the therapeutic or surrogate antibody, and the other is administered 5 minutes-5 hours prior to administration of the therapeutic or surrogate antibody. In some such embodiments, one dose of corticosteroid is administered 6-36 hours prior to administration of the therapeutic or surrogate antibody, and the other is administered 15-120 minutes prior to administration of the therapeutic or surrogate antibody. In some such embodiments, the first dose of corticosteroid is administered 16-24 hours prior to administration of the therapeutic or surrogate antibody. In some such embodiments, the second dose of corticosteroid is administered 30-60 minutes prior to administration of the therapeutic or surrogate antibody.

When the strategy for overcoming a tolerability issue is a therapeutic treatment, this may be done by administering a therapeutic agent to the second mouse, or second group of mice, in conjunction with the intravenous or intraperitoneal administration of the therapeutic or surrogate antibody to the mouse, or group of mice. In this context, in conjunction means essentially at the same time or shortly after. The second mouse, or second group of mice, is observed during a period following immediately after the administration of the therapeutic or surrogate antibody The results for the second mouse, or second group of mice, are compared to the results for the control mouse, or control group of mice, which has not received the therapeutic agent. A decreased display of the macroscopic symptoms for the second mouse, or the second group of mice, compared to the control mouse, or control group of mice, or no display of the macroscopic symptoms at all for the second mouse, or second group of mice, during the period is an indication that administration of the therapeutic agent can be used to prevent or mitigate the tolerability issue that would be associated with intravenous administration of the therapeutic antibody molecule to a human.

The therapeutic agent tested this way may be any agent or drug that is known to reverse or manage adverse events. Immune modulatory agents, such as antibodies, for example an anti-IL-6 antibody, known for use against cytokine release syndrome (Frey N V, Porter D L. Cytokine release syndrome with novel therapeutics for acute lymphoblastic leukemia. Hematology Am Soc Hematol Educ Program. 2016; 2016:567-572), or immune suppressive and/or anti-inflammatory agents, such as corticosteroids or anti-histamine.

When the strategy for overcoming a tolerability issue is a different route of administration, the therapeutic or surrogate antibody is administered to the second mouse, or second group of mice, by a route of administration other than intravenous or intraperitoneal administration. The second mouse, or second group of mice, is observed during a period following immediately after the administration of the therapeutic or surrogate antibody The results for the second mouse, or second group of mice, are compared to the results for the control mouse, or control group of mice, which has received therapeutic or surrogate antibody by intravenous or intraperitoneal administration. A decreased display of the macroscopic symptoms for the second mouse, or the second group of mice, compared to the control mouse, or control group of mice, or no display of the macroscopic symptoms at all for the second mouse, or second group of mice, during the period is an indication that administration of the therapeutic antibody molecule to a human by a route of administration other than intravenous or intraperitoneal can be used to prevent or mitigate the tolerability issue that would be associated with intravenous administration of the therapeutic antibody molecule to a human.

The administration route tested this way may be any route that is known to the skilled person and that is suitable for administration of the therapeutic antibody molecule to humans and also workable for administration of the therapeutic or surrogate antibody to mice.

In some embodiments of these further aspects of the invention, the different route of administration, i.e. the route of administration other than intravenous or intraperitoneal administration, is subcutaneous administration. The therapeutic or surrogate antibody should then be prepared or formulated for subcutaneous administration to the second mouse, or second group of mice.

When the strategy for overcoming a tolerability issue is using a modified format of the therapeutic or surrogate antibody, this modified format of the therapeutic or surrogate antibody is administered intravenous or intraperitoneal to the second mouse. The second mouse, or second group of mice, is observed during a period following immediately after the administration of the modified therapeutic or surrogate antibody The results for the second mouse, or second group of mice, are compared to the results for the control mouse, or control group of mice, which has received the unmodified therapeutic or surrogate antibody by intravenous or intraperitoneal administration. A decreased display of the macroscopic symptoms for the second mouse, or the second group of mice, compared to the control mouse, or control group of mice, or no display of the macroscopic symptoms at all for the second mouse, or second group of mice, during the period is an indication that administration of the modified therapeutic antibody molecule to a human can be used to prevent or mitigate the tolerability issue that would be associated with intravenous administration of the therapeutic antibody molecule to a human.

The modification of the therapeutic antibody molecule tested this way may be any modification known or unknown to give rise to less or less severe toxic events in humans. For example, if a therapeutic antibody molecule that is found to be associated with a tolerability issue in connection with intravenous administration to a human is an antibody that engages Fc receptors, such a modification may be to alter the antibody so that it does not engage Fc receptors or so that it has impaired or abrogated FcγR binding compared to the therapeutic non-modified antibody, while the Fv variable sequence of the modified antibody remains the same as the therapeutic antibody molecule. As mentioned above, the Fc of the surrogate antibody should be chosen to match the therapeutic antibody molecule's Fc with respect to binding/non-binding (or engagement/non-engagement) of FcγR-binding and function.

In some embodiments of these further aspects of the invention, the modification is one that leads to increased engagement of Fc receptors.

It is possible to test more than one of the above strategies, or several variants of one or more than one of the above strategies, at the same time by including further mice, or groups of mice, which one mouse, or group of mice for each strategy or each variant of a strategy.

Described herein in these further aspects of the invention is also a corticosteroid for use in a dosing regimen to prevent or mitigate a tolerability issue in connection with intravenous administration of a therapeutic antibody molecule to a subject, as well as a method for preventing or mitigating a tolerability issue in connection with intravenous administration of a therapeutic antibody molecule to a subject comprising a dosing regimen for administration of a corticosteroid to the subject.

The therapeutic antibody molecule in these further aspects of the invention may be one that has been predicted to be associated with a tolerability issue in connection with intravenous administration to a human using the predictive method described above. In addition, or alternatively, the predictive method described above may have been used to predict that pre-treatment with a corticosteroid in combination with administration of the therapeutic antibody molecule to a human is likely to prevent or mitigate the tolerability issue that otherwise would be associated with intravenous administration of the therapeutic antibody molecule to a human.

The dosing regimen comprises administration of the corticosteroid to a subject in at least two doses prior to intravenous administration of the therapeutic antibody molecule. One dose (“the first dose”) of the corticosteroid is administered 10-48 hours prior to start of the administration of therapeutic antibody molecule and one dose (“the second dose”) of the corticosteroid is administered 5 minutes-5 hours prior to the start of administration of the therapeutic antibody molecule. In addition to these two doses, it is possible to use further doses, such as one dose prior to “the first dose”, and/or one dose between “the first dose” and “the second dose”. Often, a patient is given several administrations of a therapeutic antibody molecule during a whole therapy. The two doses of corticosteroid may then be administered to the patient in connection with one or several administrations of the therapeutic antibody molecule. Preferably, the two doses are given to the patient in connection with each administration of the therapeutic antibody molecule.

In some cases, the first dose of corticosteroid is given 6-36 hours prior to start of administration of the therapeutic antibody molecule and the second dose of corticosteroid is given immediately prior to start of administration of the therapeutic antibody molecule. In this context, immediately prior means approximately 15-120 minutes prior to start of administration of the therapeutic antibody molecule.

In some cases, the first dose of corticosteroid is given 8-30 hours prior to start of administration of the therapeutic antibody molecule.

In some cases, the first dose of corticosteroid is given 16-24 hours prior to start of administration of the therapeutic antibody molecule.

In some cases, the second dose of corticosteroid is given 30-60 minutes prior to start of administration of the therapeutic antibody molecule.

In some cases, the first dose of corticosteroid is given 16-24 hours prior to start of administration of the therapeutic antibody molecule and the second dose of corticosteroid is given 30-60 minutes prior to start of administration of the therapeutic antibody molecule.

In some case, the dosing regimen comprises administration of at least two doses of the corticosteroid prior to each infusion of the antibody during the course of antibody therapy.

The corticosteroid used is preferably a potent corticosteroid, and more preferably a corticosteroid with as high potency as possible or as available. Examples of such corticosteroids are dexamethasone and betamethasone. It is possible to use either dexamethasone or betamethasone, or a a combination of dexamethasone and betamethasone.

In some cases when dexamethasone is used, the first dose is 4-20 mg. In some cases when dexamethasone is used, the second dose is 4-25 mg. In some cases when dexamethasone is used, the first dose is 4-20 mg and second dose is 4-25 mg. In some cases when dexamethasone is used, the first dose is 10-12 mg.

In some cases when dexamethasone is used, the second dose is 20 mg. In some cases when dexamethasone is used, the first dose is 10-12 mg and the second dose is 20 mg. In some cases when betamethasone is used, the first dose is 3.2-16 mg. In some cases when betamethasone is used, the second dose is 3.2-20 mg.

In some cases when betamethasone is used, the first dose is 3.2-16 mg and the second dose is 3.2-20 mg. In some cases when betamethasone is used, the first dose is 8-9.6 mg. In some cases when betamethasone is used, the second dose is 16 mg. In some cases when betamethasone is used, the first dose is 8-9.6 mg and the second dose is 16 mg.

In some cases, the dosing regimen comprises administration of an antihistamine in addition to the at least two administrations of a corticosteroid. In some cases, the antihistamine is administered 10 minutes-24 hours prior to start of administration of the therapeutic antibody molecule. In some cases, the antihistamine is administered 30-60 minutes prior to start of administration of the therapeutic antibody molecule.

The therapeutic antibody molecule, for with the corticosteroid is used to prevent or mitigate a tolerability issue in connection with intravenous administration is in some cases an Fc receptor binding antibody. In some cases, it is an anti-FcγRIIB antibody. In some cases, it is the anti-FcγRIIB antibody is the antibody having a light chain with SEQ ID No: 1 and a heavy chain with SEQ ID No: 2.

In a thirteenth aspect of the invention, described herein is also a therapeutic antibody molecule for use in the treatment of cancer, wherein the therapeutic antibody molecule is formulated for subcutaneous administration in order to prevent or mitigate a tolerability issue that would occur in connection with intravenous administration of the therapeutic antibody molecule to a subject, as well as a method for treatment of cancer comprising subcutaneous administration of a therapeutic antibody instead of intravenous administration in order to prevent or mitigate a tolerability issue.

In some cases, the therapeutic antibody molecule formulated for subcutaneous administration is an anti-FcγRIIB antibody. In some such cases, the therapeutic antibody molecule is the antibody having a light chain with SEQ ID No: 1 and a heavy chain with SEQ ID No: 2.

In a fourteenth aspect of the invention, described herein is also a modified format of a therapeutic antibody molecule for use in the treatment of cancer, wherein the modification of the therapeutic antibody molecule is made in order to prevent or mitigate a tolerability issue that would occur in connection with intravenous administration of the therapeutic antibody molecule to a subject, as well as a method for treatment of cancer comprising administration of such a modified format of a therapeutic antibody.

The modification of the therapeutic antibody molecule used may be any modification known to give rise to less or less severe toxic events in humans.

As mentioned above, the modification of the therapeutic antibody may also be a modification previously unknown to give rise to less or less severe toxic events in humans that has been tested with the predictive model described herein and found to be useful in order to prevent or mitigate a tolerability issue that would occur in connection with intravenous administration of the therapeutic antibody molecule to a subject.

As mentioned above, one example is that if a therapeutic antibody molecule that is found to be associated with a tolerability issue in connection with intravenous administration to a human is an antibody that engages Fc receptors, a modification used in the present context may be to alter the antibody so that it does not engage Fc receptors or so that it has impaired or abrogated FcγR binding compared to the therapeutic non-modified antibody, while the Fv variable sequence of the modified antibody remains the same as the therapeutic antibody molecule.

Thus, in some such cases, the therapeutic antibody molecule is an Fc receptor binding antibody and the modified format is an antibody having the same Fv variable sequence but having impaired or abrogated FcγR binding compared with the therapeutic antibody molecule. In some case, the therapeutic antibody is an Fc receptor binding antibody anti-FcγRIIB antibody, and in some such cases, the modified format is anti-FcγRIIB antibody is the antibody having a light chain with SEQ ID No: 1 and a heavy chain with SEQ ID No: 195.

In some cases the anti-FcgRIIB antibody is used as single agent. In other cases it is used to enhance activity, or overcome resistance, to other therapeutic antibodies whose activity is modulated by FcgRs e.g. anti-CD20 or anti-PD-1.

With reference to combined treatment anti-CD20 antibodies, anti-FcgRIIB may be used to in treatment of both cancer and inflammatory/autoimmune disease where anti-CD20 antibodies have been approved for therapy. The term subject used herein refers to a human who has been diagnosed as having a specific disease. Herein, the terms subject and patient are used interchangeably.

In some cases, the subject has been diagnosed with a cancer. In some such cases, the cancer is a B-cell malignancy. In some such cases, the cancer is selected from the group consisting of non-Hodgkin lymphoma, such as follicular lymphoma, diffuse large B cell lymphoma, mantle cell lymphoma, or chronic lymphocytic leukemia.

In some cases, the tolerability issue that is prevented or mitigated is thrombocytopenia (decrease of platelets). In some such cases, it is transient thrombocytopenia.

In some cases, the tolerability issue that is prevented or mitigated is cytokine release syndrome. In some such cases, it is transient cytokine release.

In some cases, the tolerability issue that is prevented or mitigated is elevated liver enzymes. In some such cases, it is elevated levels of aspartate aminotransferase (AST) and/or elevated levels of alanine aminotransferase (ALT).

In a fifteenth aspect of the invention, there is provided a therapeutic antibody molecule for use in the treatment of cancer, an autoimmune disease, an inflammatory disease, an immunological disease, and/or an infectious disease, wherein the therapeutic antibody molecule is an anti-FcγRIIB antibody, and wherein the therapeutic antibody molecule is formulated for subcutaneous administration.

In a sixteenth aspect of the invention, there is provided a therapeutic antibody molecule in the manufacture of a medicament for use in the treatment of cancer, an autoimmune disease, an inflammatory disease, an immunological disease, and/or an infectious disease, wherein the therapeutic antibody molecule is an anti-FcγRIIB antibody having a light chain with SEQ ID No: 1 and a heavy chain with SEQ ID No: 2, and wherein the medicament is formulated for subcutaneous administration.

In a seventeenth aspect of the invention, there is provided a pharmaceutical formulation comprising a therapeutic antibody molecule, wherein the therapeutic antibody molecule is an anti-FcγRIIB antibody having a light chain with SEQ ID No: 1 and a heavy chain with SEQ ID No: 2, and wherein the pharmaceutical formulation comprises a pharmaceutically acceptable diluent or excipient, and is formulated for subcutaneous administration.

Preferably, the therapeutic antibody in these aspects of the invention is an Fc receptor binding antibody. More preferably, the therapeutic antibody is an anti-FcγRIIB antibody.

In alternative embodiments of these aspects of the invention, the therapeutic antibody molecule is as described herein in any of the previous aspects of the invention herein.

However, in a preferred embodiment, the pharmaceutical composition comprises a therapeutic antibody molecule having a light chain with SEQ ID No:1 and a heavy chain with SEQ ID No:2 (which antibody is denoted as BI-1206, as described herein). Preferably, the therapeutic antibody molecule comprises a light chain with SEQ ID No:1, and a heavy chain with SEQ ID No:2, and constant regions with SEQ ID No:202 and 203.

Thus, the invention also provides the following:
a therapeutic antibody molecule for use in the treatment of cancer, wherein the therapeutic antibody molecule is an anti-FcγRIIB antibody having a light chain with SEQ ID No: 1 and a heavy chain with SEQ ID No: 2, and wherein the therapeutic antibody molecule is formulated for subcutaneous administration;
use of a therapeutic antibody molecule in the manufacture of a medicament for use in the treatment of cancer, wherein the therapeutic antibody molecule is an anti-FcγRIIB antibody having a light chain with SEQ ID No: 1 and a heavy chain with SEQ ID No: 2, and wherein the therapeutic antibody molecule and/or medicament is formulated for subcutaneous administration;
a pharmaceutical formulation comprising a therapeutic antibody molecule, wherein the therapeutic antibody molecule is an anti-FcγRIIB antibody as defined herein (and is preferably an anti-FcγRIIB antibody having a light chain with SEQ ID No: 1 and a heavy chain with SEQ ID No: 2), and wherein the pharmaceutical formulation comprises a pharmaceutically acceptable diluent or excipient, and is formulated for subcutaneous administration.

Preferably, the therapeutic antibody molecule for use, the use of a therapeutic antibody molecule, or the pharmaceutical formulation according to the above aspects of the invention (including the fifteenth, sixteenth and seventeenth aspects of the invention) are for treatment of cancer.

It will be appreciated that the pharmaceutical formulation of these aspects of the invention comprises a therapeutically effective amount of the therapeutic antibody. Preferably, the therapeutic antibody is present at a concentration of between about 90 mg/mL and about 220 mg/mL. For example, the therapeutic antibody may be present at a concentration of about 90 mg/mL, or about 100 mg/mL, or about 110 mg/mL, or about 120 mg/mL, or about 130 mg/mL, or about 140 mg/mL, about 150 mg/mL, or about 160 mg/mL, or about 170 mg/mL, about 180 mg/mL, or about 190 mg/mL, or about 200 mg/mL, about 210 mg/mL, or about 220 mg/mL. Particularly preferred is a concentration of about 150 mg/mL.

It is preferred that the pharmaceutical formulation of these aspects of the invention is sterile.

In a preferred embodiment, the pharmaceutical formulation of these aspects of the invention further comprises between about 5 mM and about 20 mM acetate, for example, about 10 mM acetate, or about 15 mM acetate. Particularly preferred is about 5 mM acetate.

In a preferred embodiment, the pharmaceutical formulation of these aspects of the invention further comprises between about 50 mM and about 250 mM NaCl, for example, about 60 mM NaCl, or about 70 mM NaCl, or about 80 mM NaCl, or about 90 mM NaCl, or about 100 mM NaCl, or about 110 mM NaCl, or about 120 mM NaCl, or about 130 mM NaCl, or about 140 mM NaCl, or about 150 mM NaCl, or about 160 mM NaCl, or about 170 mM NaCl, or about 180 mM NaCl, or about 190 mM NaCl, or about 200 mM NaCl, or about 210 mM NaCl, or about 220 mM NaCl. Particularly preferred is about 110 mM NaCl.

In a preferred embodiment, the pharmaceutical formulation of these aspects of the invention further comprises about 0.05% (w/v) Polysorbate 20, such as Tween 20 (Polysorbate) EMPROVE® ESSENTIAL Ph Eur,JPE,NF from Merck/Sigma Aldrich with catalogue No. 8.17072.1000.

In a preferred embodiment, the pharmaceutical formulation of these aspects of the invention is at a pH of between about pH 5.0 and about pH 5.8, for example about pH 5.1, or about pH 5.2, or about pH 5.3, or about pH 5.4, or about pH 5.5, or about pH 5.6, or about pH 5.7. Particularly preferred is a pH of about pH 5.8.

In a particularly preferred embodiment, the pharmaceutical formulation of these aspects of the invention comprises or consists of:
the therapeutic antibody at a concentration of 150 mg/mL;
5-5 mM acetate;
110 mM NaCl;
0.05% (w/v) Polysorbate 20; and
pH 5.8.

In an eighteenth aspect of the invention, there is provided a method for the treatment of cancer, an autoimmune disease, an inflammatory disease, an immunological disease, and/or an infectious disease in a subject, the method comprising the step of administering to the subject a therapeutic antibody molecule, wherein the therapeutic antibody molecule is an Fc receptor binding antibody, and wherein the therapeutic antibody molecule is formulated for subcutaneous administration.

Preferably, in the eighteenth aspect of the invention, the Fc receptor binding antibody is an anti-FcγRIIB antibody. More preferably, the Fc receptor binding antibody is an anti-FcγRIIB antibody having a light chain with SEQ ID No: 1 and a heavy chain with SEQ ID No: 2.

Thus, in a preferred embodiment, the invention provides:
a method for the treatment of cancer, an autoimmune disease, an inflammatory disease, an immunological disease, and/or an infectious disease in a subject, the method comprising the step of administering to the subject a therapeutic antibody molecule, wherein the therapeutic antibody molecule is an anti-FcγRIIB antibody having a light chain with SEQ ID No: 1 and a heavy chain with SEQ ID No: 2, and wherein the therapeutic antibody molecule is formulated for subcutaneous administration. It will be appreciated that, in the eighteenth aspect of the invention, the therapeutic antibody is preferably administered to the subject by a subcutaneous route of administration.

In a nineteenth aspect of the invention, there is provided a method for the treatment of of cancer, an autoimmune disease, an inflammatory disease, an immunological disease, and/or an infectious disease in a subject, the method comprising the step of administering to the subject a pharmaceutical formulation of the seventeenth aspect of the invention. It will be appreciated that, in the nineteenth aspect of the invention, the pharmaceutical formulation is preferably administered to the subject by a subcutaneous route of administration. It is preferred that the method is for the treatment of cancer.

Preferred, non-limiting examples which embody certain aspects of the invention will now be described, with reference to the following figures and examples:

### DESCRIPTION OF THE FIGURES

FIG. 1: A mouse model that recapitulates BI-1206 tolerability profile. When the murine surrogate anti-CD32b antibody (AT-130-2 IgG2a) is injected into wildtype C57/BL6 mice intravenously (i.v.) or intraperitoneally (i.p.) the mice display reactions that recapitulates the BI-1206 tolerability profile in the clinic. A. shows the % of mice displaying macroscopic IRRs such as, isolation, decreased activity, impaired balance, piloerection, hunching followed by un-natural body posture after injection. When titrating the i.v. dose the same timing and severity of macroscopic symptoms is seen down to 10 mg (0.5 mg/kg). However, at 4 mg (0.2 mg/kg) no IRRs were seen. When administrating 200 mg (10 mg/kg) i.p. a delay in IRRs onset was seen in comparison with i.v. injection with the IRRs appearing 20-30 minutes post injection. When increasing the i.p. dose to 400 mg (20 mg/kg) the onset of IRRs was still delayed compared to the i.v. injection route however, all mice displayed IRRs to the same extent and grade as 200 mg i.v. All mice had fully recovered 1 h post injection. B. PLT (platelet counts in blood) were analysed in fresh blood using a Vetscan shows that mice displaying IRRs (gray bars) also display a decrease in platelet counts. C. Blood samples were also analyzed for AST showing an increase in the group receiving 200 ug anti-CD32b antibody i.v. D. shows IL6 levels in blood of mice injected i.p. with 200 ug anti-CD32b antibody over time post injection. A peak is seen 1 h post injection and levels are back to normal 8 h post injection (gray area). A similar pattern was seen for IL-5, IL-10, KC/GRO, TNF-α.

FIG. 2: Pre-medication with two doses of corticosteroids dose-dependently block or reduce IRRs in vivo. Mice were either pretreated with: A 40 mg/kg or B 10 mg/kg Betamethasone 24 h and 1 h (premed) pre i.v. injection of 10 mg/kg AT-130-2 IgG2a. Mice were bled 20 minutes post injection. The blood was analyzed for platelet counts. Premedication with 10 mg/kg Betamethasone did not completely inhibited IRRs or decrease in platelet count B (striped bars) indicating that lowering the dose of premedication might reduce its potential to inhibit IRRs and platelet decreases.

FIG. 3: Split dose—a small pre-dose of Ab lowers the severity of IRRs and platelet decrease. A. Split-dosing was initiated with 8 ug/mouse of mouse anti-CD32B AT-130-2 IgG2a i.v. followed by a bulk-dose of 200 ug/mouse 1 h later. In parallel mice were injected with only the bulk-dose. IRRs were studied (and visualized in the figure according to the grading system in B) and platelet counts, and body temperature was measured and compared. A small pre-dose of AT-130 (8 ug) lowers the severity/protects against IRR's, platelet decrease, and body temperature decrease when giving a large/main dose (200 ug). Gray area indicates normal range of PLT (platelet counts in blood) and body temperature. The pre-dose needs to be 8 ug (a dose where IRRs are seen in 50% of the mice C), lower doses are not protective.

FIG. 4: Combined pre-medication with steroids and antibody split-dosing is needed for full (antibody) tolerance and protection against IRRs. Split-dosing was initiated either: A 24 h post suboptimal corticosteroid treatment (10 mg/kg) or B without corticosteroid pretreatment with 8 ug/mouse of mouse anti-CD32B AT-130-2 IgG2a i.v. followed by a bulk-dose of 200 ug/mouse 1 h later. In parallel, mice were injected with only the bulk-dose. IRRs were studied and platelet counts, and body temperature was measured and compared. The small pre-dose of AT-130 (8 ug) is well tolerated if a “low” dose of corticosteroids (10 mg/kg) is given 24 h before. The suboptimal pre-med together with the small pre-dose of AT-130 (8 ug) fully protects against IRR's and platelet decrease associated with a large Ab dose (200 ug). Suboptimal dose corticosteroids (10 mg/kg) is not protective itself. Gray area indicates normal range of PLT and body temperature.

FIG. 5: Pre-medication with several different clinically relevant substances does not inhibit IRRs associated with AT-130-2 IgG2a administration. In order to evaluate if pre-treatment with substances generally used in the clinic to treat IRRs could inhibit IRRs in this model, mice were pre-treated with anti-PAF, anti-IL6, anti-histamine or with a leukotriene-antagonist. These pre-medications were given i.p 1 h prior to injection i.v of mouse anti-CD32B AT-130-2 IgG2a as a bulk dose of 200 ug/mouse. Mice were observed for macroscopic IRRs such as isolation, mobility, and fur condition. None of these pre-medications could inhibit IRRs.

FIG. 6: Tolerability profile seen in human subjects with non-hodgkins B-cell lymphoma after administration of BI-1206 at doses of 70-100 mg. (A) Platelet decrease following treatment with BI-1206. The decrease is transient and most episodes have been resolved within a week and never serious or associated with bleeding. Each dot denotes a measurement and the line median. Vertical striped line indicates BI-1206 administration. (B) Platelet decrease is associated with ALT elevations. Though ALT/AST elevations have only been significant in 3/14 patients. (C) Frequency of detected cytokine elevations following treatment with BI-1206. A transient cytokine release has been detected in 7 of 8 subjects where serum/plasma has been available for analysis. The peak cytokine release is seen immediately after infusion and always gone within 24 h. (D) Kinetics of platelet decrease, ALT and cytokine elevations is exemplified by individual 504-001. Cytokines are here exemplified by IL-6.

FIG. 7: FcgRIIb receptor occupancy translates into B cell depletion. Clinical data is in line with pre-clinical data from in vivo models using hFcgRIIB transgenic mice where it has been demonstrated that a sustained receptor saturation is necessary to achieve sustained B lymphocyte depletion. Two doses of corticosteroids prior to administration anti-FcgRIIb mAb and a small pre-dose of mAb (split dose) improves tolerability (IRRs). FcgRIIb receptor occupancy on peripheral B cells (A) and peripheral B cell levels (B) in human subjects with non-hodgkins B-cell lymphoma treated with 100 mg BI-1206 monotherapy. FcgRIIb receptor occupancy (E) and peripheral B cell depletion (F) in hFcgRIIB transgenic mice after i.v. administration of increasing doses of BI-1206. (C) Grade of infusion related reactions (IRRs) in human subjects with non-hodgkins B-cell lymphoma treated with 70-100 mg BI-1206. Dark grey bars with black symbols denotes administrations without pre-medication with two doses of corticosteroids; light grey bars and open white symbols denotes administrations with pre-medication with two doses of corticosteroids. On the X-axis subject id and antibody dose is indicated. (D) Significantly lower frequency and severity of IRRs are seen after implementation of premedication with two doses of corticosteroids in the clinical protocol; black symbols dosing without two doses of corticosteroids, grey symbols dosing with two doses of corticosteroids; P<0.0001 using Mann Whitney U-test. (G) Infusion related reactions after i.v. administration of anti-FcgRIIb mAb (AT-130-2 mIgG2a) in wild type mice. Dose titration shows that at 0.4 mg/kg Ab IRRs appear in approximately 50% of the animals and at i.v. administrated doses above IRRs are seen in 100% of the animals. Pre-medication with two 40 mg/kg doses of betapred completely block IRRs whereas two 40 mg/kg doses of betapred partly block IRRs. Adding split dos to the suboptimal 10 mg/kg betapred blocks IRRs. The split dose is a small pre-dose of 0.4 mg/kg Ab followed by a large dose of 10 mg/kg Ab. (H) Improved tolerability and sustained efficacy is seen after implementation of premedication with two doses of corticosteroids in the clinical protocol. CR: complete response; PR: partial response; SD: stable disease; DLT: dose limiting toxicity.

FIG. 8. Transient FcγRIIB receptor occupancy and peripheral lymphocyte depletion after BI-1206 infusion. FIG. 8A) FcγRIIB receptor occupancy on peripheral B cells after BI-1206 infusion followed over time in subjects having received 100 mg BI-1206 (n=8). Vertical dashed lines denote BI-1206 infusions. At second and third BI-1206 infusion receptor occupancy data is only available pre-infusion. Line denotes the median. Based on preliminary data. FIG. 8 B) Peripheral lymphocytes after BI-1206 infusion followed over time in subjects having received 100 mg BI-1206 (n=9). Vertical dashed lines denote BI-1206 infusions. Line denotes the median. Based on preliminary data.

FIG. 9. Transient decrease of platelets associates with increase in ALT after BI-1206 infusion. FIG. 9A) Platelet count after BI-1206 infusion followed over time in subjects having received 70-100 mg BI-1206 (n=16). Vertical dashed lines denote BI-1206 infusions. Line denotes the median. Based on preliminary data. FIG. 9 B) Fold increase of ALT versus percent platelet depletion (n=16). Changes are calculated in relation to day 1, pre-BI-1206-infusion. Based on preliminary data.

FIG. 10. Cytokine release after BI-1206 infusion. Number of patients with plasma/serum increase of different cytokines detected at end of BI-1206 infusion. To be considered as positive the value should be >10-fold increased as compared to pre-infusion and >10-fold above the upper limit of normal range (ULN). Samples for analysis have been available from 5 subjects receiving 70 mg BI-1206. Based on preliminary data.

FIG. 11. Two doses of dexamethasone relieved IRRs, platelet decrease, and transaminase increase in two subjects receiving BI-1206. Platelet count and ALT in subjects 501-001 (FIG. 11A) and 503-002 (FIG. 11 B). Vertical dashed lines denote antibody infusions. The first infusion in each subject was rituximab alone and the following BI-1206 and rituximab. Grade of IRR at each antibody infusion is indicated. 501-001 and 503-002 received 12 mg and 4 mg dexamethasone, respectively, the evening before and again 20 mg dexamethasone 30 minutes prior to the third administration of BI-1206 (70 mg) during induction therapy. None of the subjects suffered any IRRs after this premedication regimen with two doses of dexamethasone. During the previous two infusions with BI-1206, where dexamethasone (20 mg) was given only 30 minutes prior to infusion, IRRs (grade 2-3) had been experienced. In addition, in subject 501-001 and 503-002 no/low platelet decrease, and no ALT/AST increase was seen after BI-1206 administration when premedicating with two doses of dexamethasone.

FIG. 12. Macroscopic symptoms after injection of murine surrogate anti-CD32b (AT-130-2 IgG2a). The murine surrogate anti-CD32b (AT-130-2 IgG2a) was injected into wildtype C57/BL6 mice through 3 different injection routes, intravenously i.v., intraperitoneally i.p. or subcutaneously s.c. Macroscopic symptoms were seen after i.v. and i.p. injection. These macroscopic symptoms included isolation, decreased activity, impaired balance, piloerection, hunching followed by un-natural body posture and the macroscopic symptoms were scored from 0-2 based on the observations. When titrating the i.v. dose a rapid onset of IRRs was seen 5-7 minutes post injection. The same timing and severity of macroscopic symptoms was seen down to 10 μg (0.5 mg/kg). However, at 1 μg (0.05 mg/kg) no macroscopic symptoms were seen. When administrating 200 μg (10 mg/kg) i.p. a delay in onset of macroscopic symptoms was seen in comparison with i.v. injection with the macroscopic symptoms appearing 20-30 minutes post injection. In contrast to the i.v. injection route all mice in this group did not display macroscopic symptoms and the macroscopic symptoms were less severe in several mice. When increasing the i.p. dose to 400 μg (20 mg/kg) the onset of macroscopic symptoms was still delayed compared to the i.v. injection route however, all mice displayed macroscopic symptoms to the same extent and grade as 200 μg i.v. All mice had fully recovered 1 hour post injection. Finally, when administrating 200 μg to mice s.c., no macroscopic symptoms were seen (up to 24 hours post injection). When increasing the s.c. dose to 400 μg the mice remained unaffected.

FIG. 13. Pharmacokinetic profiles of AT-130-2 IgG2a in C57BL/6 mice. Observed AT-130-2 serum concentrations in mice treated with AT-130 via three different administrations routes; 200 μg AT-130-2 via i.v. injection, 200 μg AT-130-2 via i.p. injection and 400 μg AT-130-2 via s.c. injection. Presented data are the mean of 1-4 mice/dose groups. Abbreviations: h=hour(s); i.p. intraperitoneal, i.v. intravenous, s.c. subcutaneous. Note that complete and sustained FcgRIIB receptor is achieved with following s.c. dosing of AT130, although s.c. C_{Max} is lower and kinetics to obtain full saturation is slower compared with i.v. or i.p. dosing.

FIG. 14. Correlation between macroscopic symptoms (denoted IRRs in this figure) and high, rapid exposure of AT-130-2 rather than time of FcγRIIB saturation. The serum concentration of AT-130-2 IgG2a (line with dots) is plotted against the grade of macroscopic symptoms (line with squares) for the different administration routes (A: i.v., B: i.p. and C: s.c.). When comparing the serum concentration of AT-130-2 and the presumed receptor occupancy (RO) (dotted line, 10 mg/ml gives 100% receptor saturation) with the onset, severity and duration of IRRs it is clear that there is a correlation between high and rapid exposure of AT-130-2, rather than time of FcγRIIB saturation. Tolerability showing a clear pattern of s.c. >i.p. >i.v. with RO being sustained for a long period of time post recovery from macroscopic symptoms.

FIG. 15A. Timing of platelet (PLT) nadir is correlated to the route of administration and time to FcγRIIB saturation. Mice were bled at different time points post injection of AT-130-2 IgG2a and the blood was analyzed for blood platelet count (PLT) in an automated Vetcount. A nadir in platelet count was seen at the same time as onset of macroscopic symptoms after injection of AT-130-2 through both the i.v. and i.p. administration route. For the s.c. administration route where no macroscopic symptoms are seen, a moderate drop was seen 10 hours post injection correlating with the time to FcγRIIB saturation according to PK. In all cases the PLT decrease was transient and was restored to values in the normal range within 8 hours post injection. Mice presenting macroscopic symptoms are shown with filled bars.

FIG. 15B shows that transaminase increase following injection of AT-130-2 IgG2a is circumvented when using the s.c. administration route. Mice were bled post injection of AT-130-2 IgG2a and the blood was analyzed for transaminases. For the i.v. administration route an increase is seen in transaminases (AST) 1 h post onset of macroscopic symptoms (previously established as time point for a peak value in transaminases). For the s.c. administration route where no macroscopic symptoms are seen, no increase in transaminases was seen 11 hours post injection (1 h post the time of FcγRIIB saturation according to PK (10 h)).

FIG. 16: Transient platelet decrease, transaminase increase and cytokine release, after administration of AT-130-2 IgG2a. Mice were bled at different time points after i.p. injection of 200 μg AT-130-2 and the blood was analyzed for platelet counts (FIG. 16A), transaminases (FIG. 16 B) and cytokines (FIG. 16 C). A transient PLT decrease was seen with the PLT counts fully restored 8 hours post injection (FIG. 16A). An increase in transaminases (AST and ALT) with a peak 1 h post injection was the only clinical chemistry parameter affected by AT-130-2 injection (FIG. 16 B). These increases were just like the PLT decrease transient. The same transient increase was seen when AT-130-2 was injected i.v. and no increase in transaminases was detected when AT-130-2 was injected s.c. (data not shown). A panel of cytokines including the analytes IFN-γ, IL-1β, IL-2, IL-4, IL-5, IL-6, IL-10, IL-12p70, KC/GRO, TNF-α were analyzed at different time points post injection of 200 mg i.p. Of the analyzed cytokines IL-5, IL-6, IL-10, KC/GRO, TNF-α showed a transient increase with them all except for IL-5 peaking 1-3 hours post injection (FIG. 16 C). IL-5 showed a delayed peak 3-8 hours post injection (FIG. 16 C). These are the same cytokines that have been indicated to increase in some patients in the clinical studies with BI-1206.

FIG. 17. Premedication with 2 doses of corticosteroids inhibits macroscopic symptoms associated with AT-130-2 IgG2a administration. Mice were either pretreated with 40 mg/kg betamethasone 24 hours and 1 hour or left untreated pre i.v. injection of 10 mg/kg AT-130-2 IgG2a. Mice were observed for macroscopic symptoms (FIG. 17A) and bleed 20 minutes post injection. The blood was analyzed for (FIG. 17 B) platelet counts, (FIG. 17 C) transaminases and cytokines. Premedication with betamethasone completely inhibited the macroscopic symptoms (FIG. 17A), decrease in platelet count (FIG. 17 B) and reduced the transaminases increase (FIG. 17 C). The transient cytokine release seen after i.v. injection of AT-130-2 was also inhibited by the premedication (data not shown). The same results were seen when using dexamethasone (data not shown).

FIG. 18. The dose of premedication is of importance. Mice were either pretreated with 40 mg/kg or 10 mg/kg Betamethasone 24 hours and 1 hour (premed) pre i.v. injection of 10 mg/kg AT-130-2 IgG2a. Mice were observed for macroscopic symptoms (FIG. 18A) and bleed 20 minutes post injection. The blood was analyzed for (FIG. 18 B) platelet counts. Premedication with 10 mg/kg Betamethasone did not completely inhibited macroscopic symptoms (FIG. 18A) or decrease in platelet count (FIG. 18 B) indicating that lowering the dose of premedication might reduce its potential to inhibit macroscopic symptoms and platelet decreases.

FIG. 19. Steroid premedication 1 hour prior to infusion is not sufficient to inhibit IRRs. Mice were either pretreated with 40 mg/kg Betamethasone 24 hours, 1 hour or 24 hours+1 hour pre i.v. injection of 10 mg/kg AT-130-2 IgG2a. Mice were observed for macroscopic symptoms (FIG. 19A) and bleed 10⁻²⁰ minutes post injection. The blood was analyzed for platelet counts (FIG. 19 B). Single premedication with betamethasone 1 hour post injection of AT-130-2 did not inhibited macroscopic symptoms (FIG. 19A) or platelet decrease (FIG. 19 B) and single premedication with betamethasone 24 hours post injection of AT-130-2 did only reduce the macroscopic symptoms to a grade 1. This indicates that two doses of steroid treatment are needed to completely inhibit tolerability issues.

FIG. 20. Premedication with anti-histamine is not enough to inhibit tolerability issues associated with AT-130-2 IgG2a administration. Mice were either pretreated with anti-histamine alone or with 40 mg/kg Betamethasone (24 hours and 1 hour)+/−anti-histamine pre i.v. injection of 10 mg/kg AT-130-2 IgG2a. Mice were observed for macroscopic symptoms (FIG. 20A) and bleed approximately 20 minutes post injection. The blood was analyzed for platelet counts (FIG. 20 B). Premedication with anti-histamine alone did not inhibit macroscopic symptoms (FIG. 20A) but did seem to improve decrease in platelet counts (FIG. 20 B). The addition of anti-histamine to 40 mg/kg Betamethasone (24 hours and 1 hour) pre i.v. injection of 10 mg/kg AT-130-2 IgG2a did not affect macroscopic symptoms or platelet counts. The same results were seen when using three different types of anti-histamines (Zyrlex, Zantac or Au, data not shown).

FIG. 21 shows that several but not all murine surrogate antibodies induce IRRs. The murine surrogates anti-CD32b antibody (AT-130-2 mIgG2a), anti-CSFR1 (AFS98 rIgG2a), anti-EGFR (7A7 mIgG2a), anti-CD40 (FGK4.5 rIgG2a) and anti-FcγRIII (AT154-2 mIgG2a) were injected into wildtype C57/BL6 mice intravenously i.v. IRRs was seen after injection of anti-CD32b, anti-CD40 and anti-FcγRIII. The IRRs included isolation, decreased activity, impaired balance, piloerection, hunching followed by un-natural body posture and the IRRs were scored from 0-2 based on the observations. No IRRs were seen for anti-EGFR or anti-CFSR1. When mice were pretreated with 40 mg/kg betamethasone 24 h and 1 h pre-injection of anti-CD32b or anti-CD40 no IRRs were seen (anti-FcγRIII was not evaluated with premedication). Indicating that premedication can inhibit IRRs related to different antibodies and targets.

FIG. 22 shows that antibodies inducing IRRs also induce platelet decrease. The murine surrogates anti-CD32b antibody (AT-130-2 mIgG2a), anti-CD40 (FGK4.5 rIgG2a) and anti-FcγRIII (AT154-2 mIgG2a) were injected into wildtype C57/BL6 mice intravenously i.v. Platelet counts were analyzed in fresh blood 20 min post injection using a Vetscan (Vetscan HM5 Abaxis, Triolab). A decrease in platelet counts was seen after injection of anti-CD32b, anti-CD40 and anti-FcγRIII. When mice were pretreated with 40 mg/kg betamethasone 24 h and 1 h pre-injection of anti-CD32b or anti-CD40 no platelet decrease were seen (anti-FcγRIII was not evaluated with premedication). Indicating that premedication can inhibit platelet decrease related to different antibodies and targets.

Specific, non-limiting examples which embody certain aspects of the invention will now be described. These examples should be read together with the brief description of the drawings provided above.

### EXAMPLES

### Example 1

### Summary

A split dosing regimen in combination with corticosteroid pre-treatment was evaluated in an in vivo model recapitulating the tolerability profile seen with BI-1206 using the BI-1206 murine surrogate AT-130-2 IgG2a. The split dosing regimen in combination with corticosteroid pre-treatment improves the tolerability profile of anti-FCyRIIb treatment. Macroscopic IRRs, and platelet counts are improved with split dosing. The time span between the first dose and the second dose does not seem to be of importance while the correct timing of pre-treatment with corticosteroids appears to be important for complete tolerance of the first dose.

### Materials & Methods

### Test Substance

The anti-mouse CD32B IgG2a clone AT130-2 was transiently expressed in HEK293 cells. The specificity of the purified research batch was demonstrated in a luminescence-based ELISA or in FACS analyses. Endotoxin-levels of antibodies were found to be <0.1 IU/mL as determined by the LAL-Amoebocyte test.

| Antibody clone | Description | Reference |
|---|---|---|
| AT-130-2 IgG2a | Mouse surrogate of | mIgG2aK-AT130 ref: uct, |
| | BI-1206 | 2019 Jun. 7, 1443: 65 |

### Mice

Six to eight weeks-old (17-20 g) female C57/BL6 and Balb C mice were obtained from Taconic or Janvier. Mice were injected i.v with mouse anti-CD32B AT-130-2 IgG2a either as a bulk dose of 200 ug/mouse or as a split-dose with 8 ug/mouse followed by 200 ug/mouse.

### Premedication

For the corticosteroid treatment, Betapred (betamethasone, VNR: 008938, Alfasigma S.P.A.) was used at 10 mg/kg which is a suboptimal dose in these mouse models.

### Split-Dosing

Split-dosing was initiated 24 h post corticosteroid treatment with 8 ug/mouse of mouse anti-CD32B AT-130-2 IgG2a i.v. followed by a bulk-dose of 200 ug/mouse 20-40 minutes later. In parallel mice were injected with only the bulk-dose.

### Animal Monitoring

Mice were monitored post injection with regards to changes in behavior and macroscopic symptoms such as isolation, mobility, and fur condition. Macroscopic IRRs scoring system of 0-2 was set up based on the observations.

| Scoring | Macroscopic symptoms |
|---|---|
| 0 | No visible symptoms |
| 1 | Isolation, decreased activity |
| 2 | Isolation, decreased activity, impaired balance, piloerection, |
| | hunching followed by un-natural body posture |

### Body Temperature

Body temperature was measured 20 min post injection of bulk dose with a mouse thermometer.

### Blood Sampling

Blood samples were collected from vena saphena 20 min post injection of bulk dose of anti-CD32B for instant blood count analysis. For liver enzyme and cytokine analysis, the mice were bled from the aorta under isoflurane anesthesia just prior to sacrifice. Samples for liver enzymes and cytokines were collected 1 h and respectively 3 h after bulk dose.

### Platelet Count

Platelet counts were analyzed in fresh blood using a Vetscan (Vetscan HM5 Abaxis, Triolab).

### Transaminases

Transaminases were analyzed shipping of frozen serum samples to (IDEXX BioResearch Vet Med Labor GmbH).

### Results and Discussion

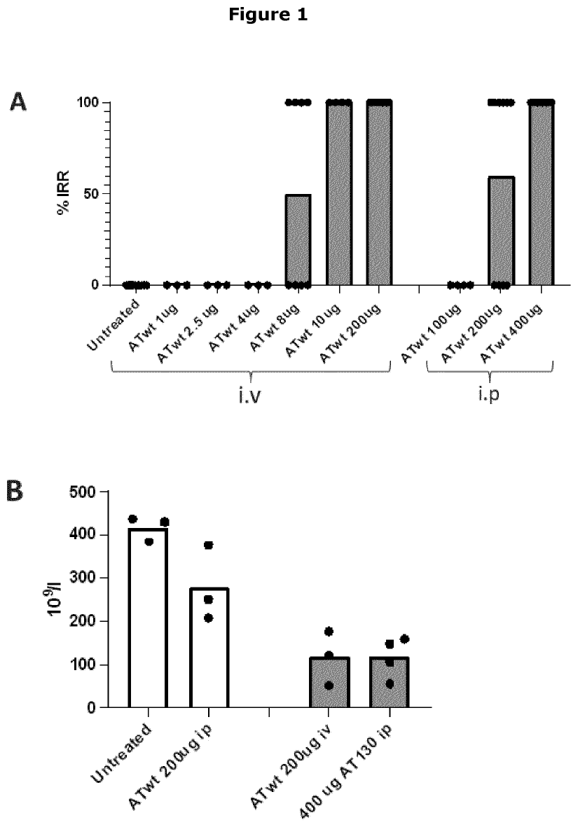

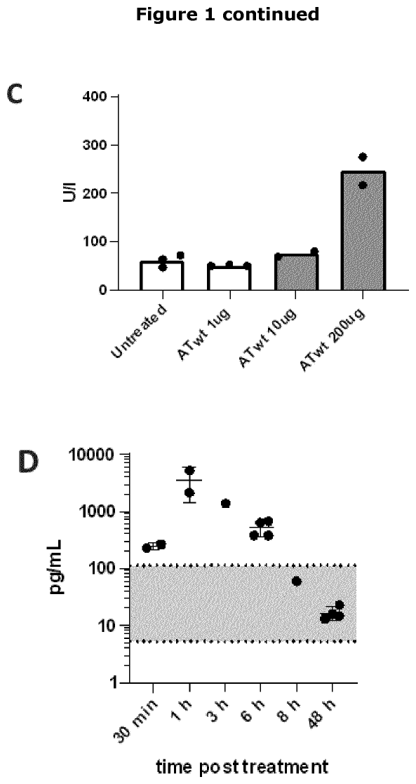

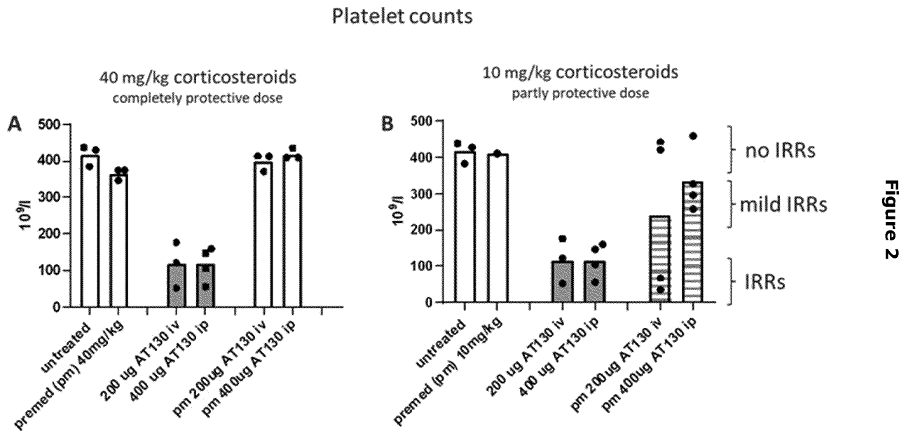

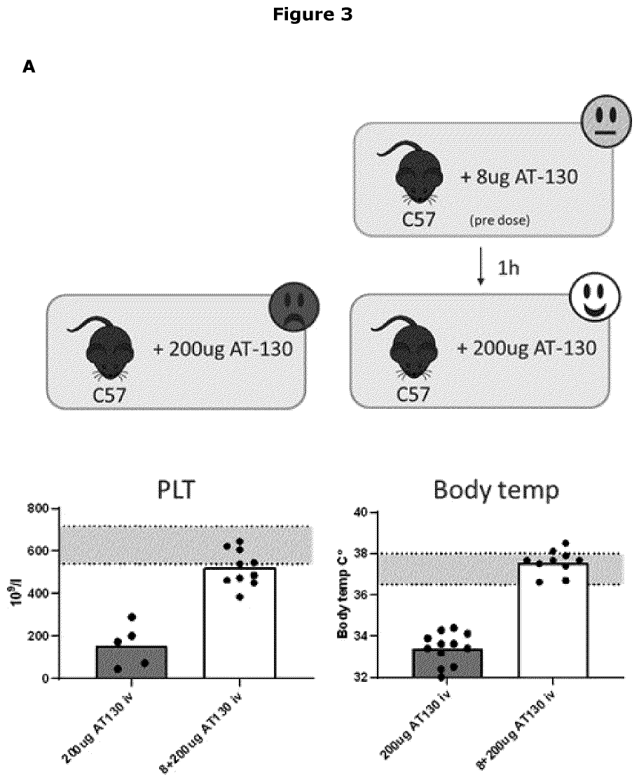

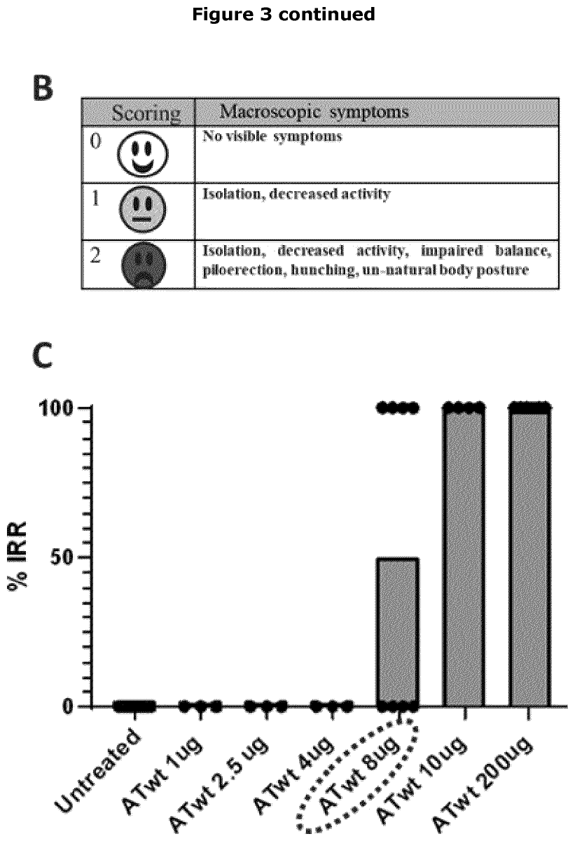

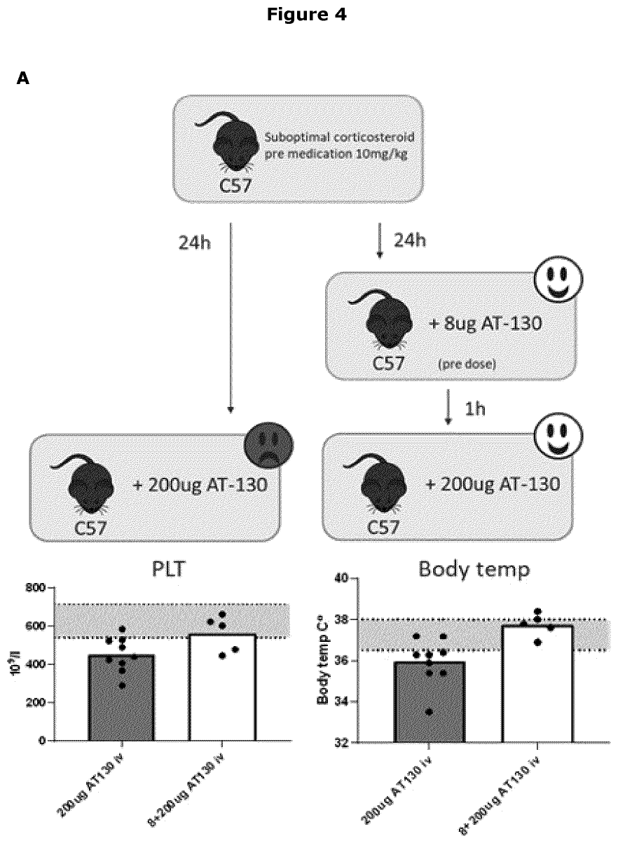

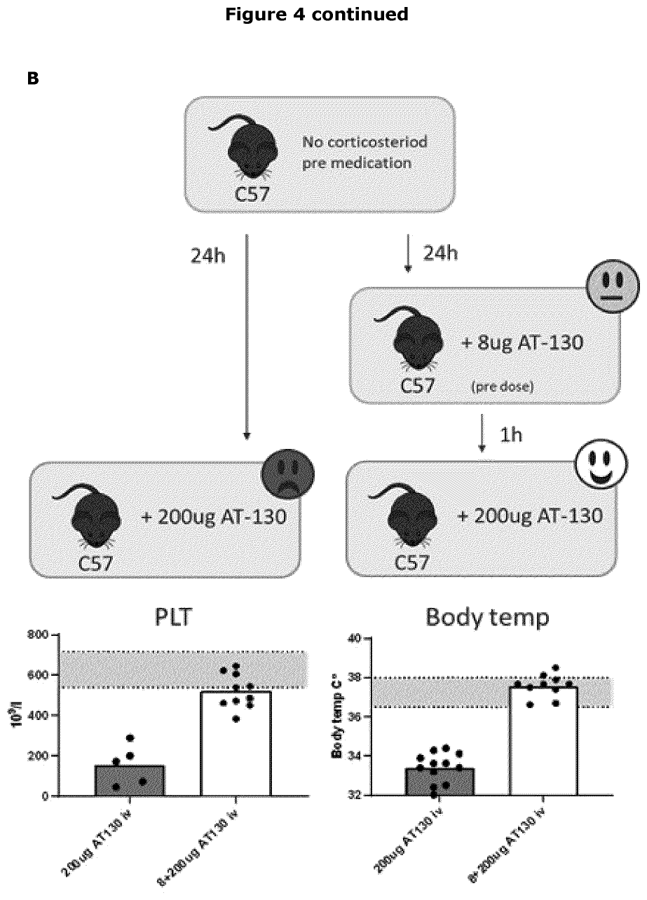

A split dosing regimen in combination with corticosteroid pre-treatment improves the tolerability profile of anti-CD32b treatment. The tolerability profile of anti-CD32b treatment alone can be seen in FIG. 1. Macroscopic IRRs and platelet counts are improved with split dosing in combination with an initial dose of corticosteroid (FIGS. 2, 3 and 4). This was evaluated in an in vivo model recapitulating the tolerability profile seen with BI-1206 using the BI-1206 murine surrogate AT-130-2 IgG2a. The time span between the first dose and the second dose does not seem to be of importance however, the correct timing of pre-treatment with corticosteroids appears important for complete tolerance of the first dose.

### Example 2

### Summary

In order to evaluate if pre-treatment with other substances (aside from corticosteroids) generally used in the clinic to treat IRRs could inhibit IRRs in this model, the inventors pre-treated mice with several other clinically relevant substances. None of the tested pre-medications could inhibit IRRs in this model, suggesting they are not useful in preventing adverse effects associated with BI-1206 administration.

### Materials and Methods

### Test Substance

The anti-mouse CD32B IgG2a clone AT130-2 was transiently expressed in HEK293 cells. The specificity of the purified research batch was demonstrated in a luminescence-based ELISA or in FACS analyses. Endotoxin-levels of antibodies were found to be <0.1 IU/mL as determined by the LAL-Amoebocyte test.

| Antibody clone | Description | Reference |
|---|---|---|
| AT-130-2 IgG2a | Mouse surrogate of | mIgG2aK-AT130 ref: uct, |
| | BI-1206 | 2019 Jun. 7, 1443: 65 |

### Mice

Six to eight week-old (17-20 g) female C57/BL6 and Balb C mice were obtained from Taconic or Janvier. Mice were injected i.v with mouse anti-CD32B AT-130-2 IgG2a either as a bulk dose of 200 ug/mouse or as a split-dose with 8 ug/mouse followed by 200 ug/mouse.

### Premedication

For the corticosteroid treatment, Betapred (betamethasone, VNR: 008938, Alfasigma S.P.A.) was used at 10 mg/kg which is a suboptimal dose in these mouse models.

Other premedications evaluated were anti-PAF (CV3988, sc-201015, Santa Cruz 20 mg/kg), anti-IL6 (clone 15A7, BE0047, Bioxcell, 10 mg/kg), anti-histamine (Zantac, VNR: 077875, GlaxoSmithKline AB, 5 mg/kg) or a leukotriene-antagonist (131064, Apoex, 4 mg/kg). These premedications were given i.p 1 h prior to injection i.v of mouse anti-CD32B AT-130-2 IgG2a as a bulk dose of 200 ug/mouse.

### Split-Dosing

Split-dosing was initiated 24 h post corticosteroid treatment with 8 ug/mouse of mouse anti-CD32B AT-130-2 IgG2a i.v followed by a bulk-dose of 200 ug/mouse 20-40 minutes later. In parallel mice were injected with only the bulk-dose.

### Animal Monitoring

Mice were monitored post injection with regards to changes in behavior and macroscopic symptoms such as isolation, mobility, and fur condition. A macroscopic IRR scoring system of 0-2 was set up based on the observations.

| Scoring | Macroscopic symptoms |
|---|---|
| 0 | No visible symptoms |
| 1 | Isolation, decreased activity |
| 2 | Isolation, decreased activity, impaired balance, piloerection, |
| | hunching followed by un-natural body posture |

### Body Temperature

Body temperature was measured 20 minutes post injection of bulk dose with a mouse thermometer.

### Blood Sampling

Blood samples were collected from vena saphena 20 minutes post injection of bulk dose of anti-CD32B for instant blood count analysis. For liver enzyme and cytokine analysis, the mice were bled from the aorta under isoflurane anesthesia just prior to sacrifice.

Samples for liver enzymes and cytokines were collected 1 h and respectively 3 h after bulk dose.

### Platelet Count

Platelet counts were analyzed in fresh blood using a Vetscan (Vetscan HM5 Abaxis, Triolab).

### Transaminases

Transaminases were analysed by shipping of frozen serum samples to (IDEXX BioResearch Vet Med Labor GmbH).

### Results and Discussion

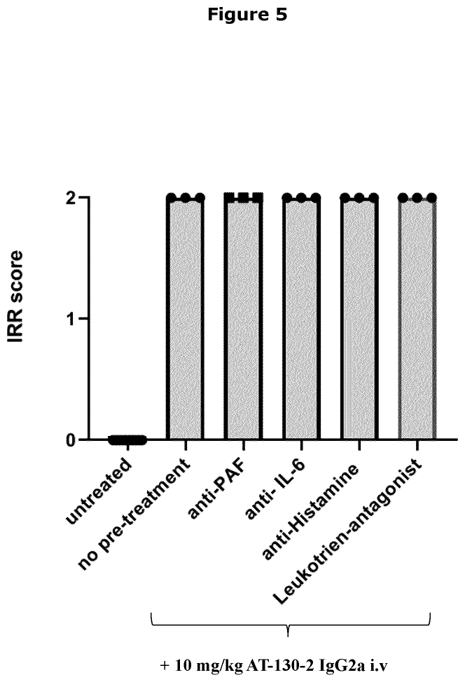

As shown in FIG. 5, none of the tested substances (anti-PAF, anti-IL-6, anti-histamine and a leukotrien-antagonist) could prevent the IRRs associated with AT-130-2 administration in this mouse model. This suggests that only corticosteroids as a pre-treatment are capable of providing the protective effect described in Example 1. This finding is surprising considering all of these substances are generally used in clinic to treat IRRs associated with other therapeutic antibodies.

### Example 3

### Summary

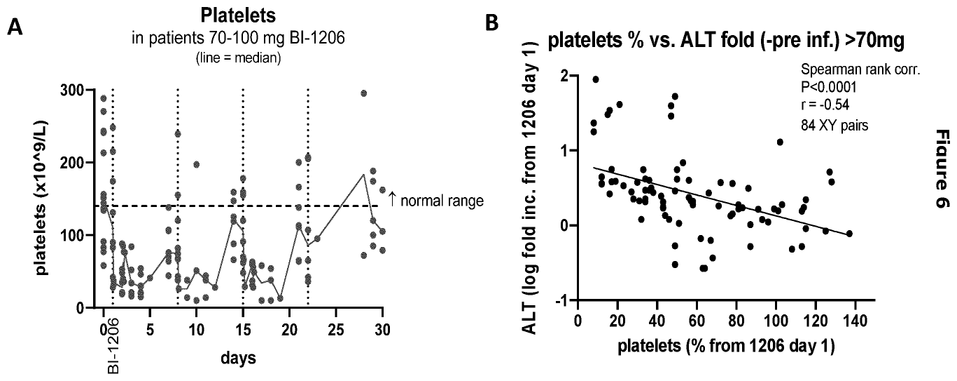

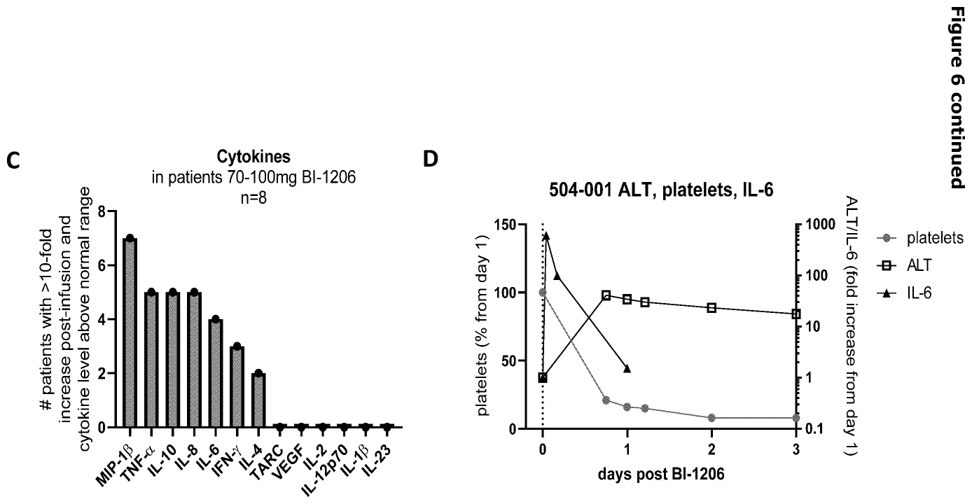

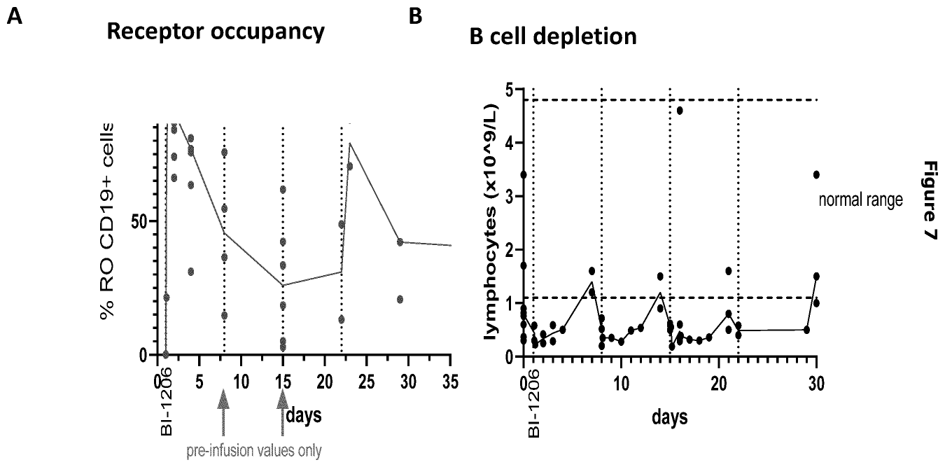

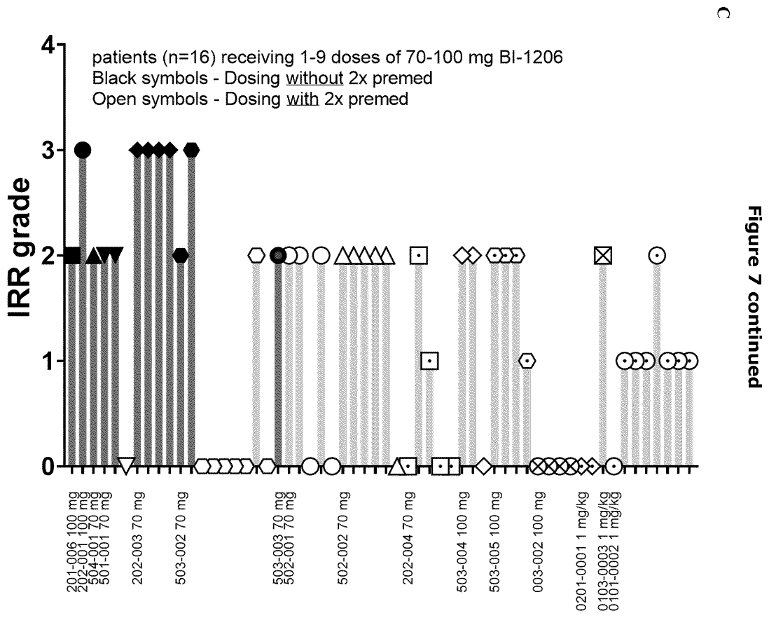

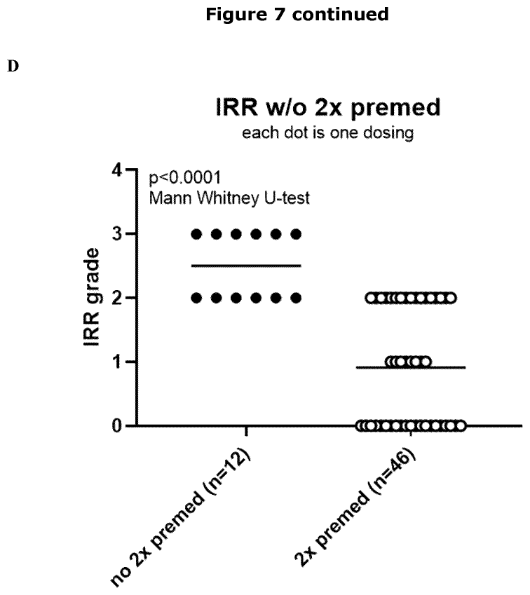

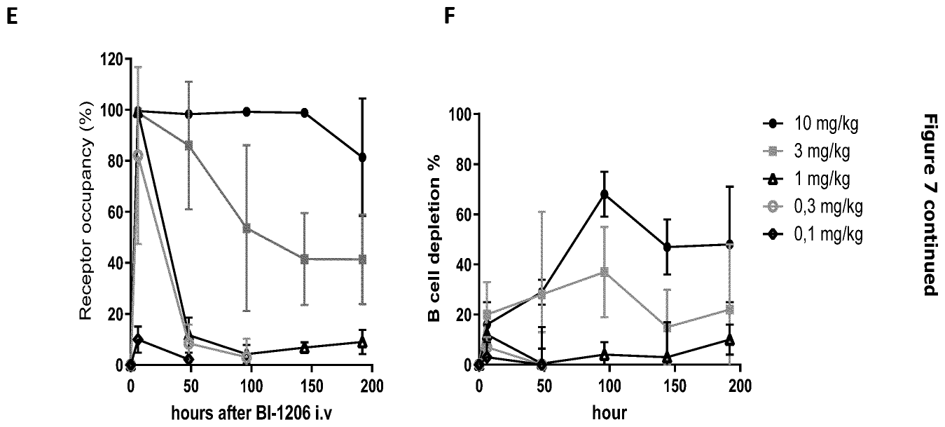

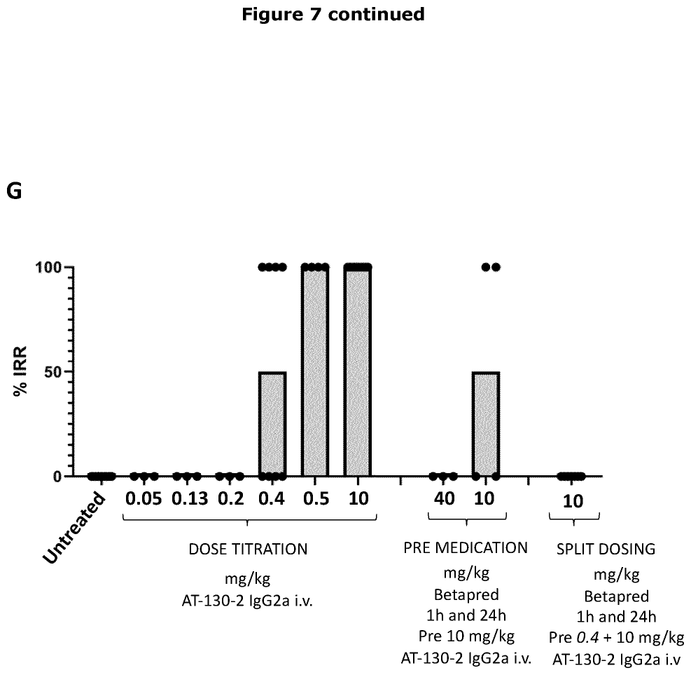

It is apparent that iv administration of BI-1206 is frequently associated with IRRs, thrombocytopenia, transient spikes in cytokines, and less frequently but in the most severe cases, increases in liver enzymes (FIG. 6). It it therefore advantageous if a dosage regimen can be used which can prevent or mitigate these adverse effects. It is also apparent that in non-Hodgkin lymphoma patients, FcγRIIb receptor occupancy translates to B cell depletion (which correlates with in vivo data from mouse models), and sustained receptor saturation is therefore important for sustained B lymphocyte depletion, however achieving such sustained high receptor occupancy required for therapeutic benefit by i.v. injection is associated with high levels of IRRs (FIG. 7).

### Materials and Methods

Platelet counts, ALT concentrations and IRR grading Platelet counts, ALT concentrations and IRR grading were obtained from clinical sites where analyzed and reported according local standard procedures. All described data from the clinical studies is preliminary, only partially quality controlled and should be considered as illustrative of the pharmacodynamic effects and tolerability associated with BI-1206.

### FcgRIIb Receptor Occupancy

The FcgRIIb receptor occupancy in humans and hFcgRIIb transgenic mice was analyzed using flow cytometry. Whole blood from was incubated with either 005-005 antibody (targeting hFcgRIIb) or anti-hCD32-AF647 antibody. 005-005 binds the same epitope as BI-1206 but with much lower affinity. In the analysis, the Geo Mean of respectively mAb (005-005 and anti-human CD32) was acquired on the CD19+ cell population. Receptor occupancy (RO) was calculated using the following equations: RO (%)=((Total Receptors-Normalized Free Receptors)*100)/Total Receptors. All replicates of 005-005 geo mean of CD19+ cells were then multiplied by the Normalization factor.

### Cytokine Analysis

For cytokine concentrations, frozen plasma samples were thawed and diluted ×2 and x8. Two parallel set of cytokines were analyzed, the proinflammatory assay with IL-6, IL-8, TNF-α, IFN-γ, IL-10, IL-2 and IL-4 (MesoScale Discovery (MSD) #K15049), and the chemokine assay with MIP-113, IL-113, IL-23, IL-12p70, TARC and VEGF (MSD #K15067). The assays followed the manufacturer's protocol as outlined briefly: 50 μL of sample and calibration standard were added to the appropriate MSD plates and incubated. Following washing, 25 μL of SULFO-TAG detection antibody mixture were added to each well of the corresponding plate. The plates were analyzed on a QuickPlex SQ120 Reader instrument (MSD) and cytokine concentration was calculated using the MSD software (Discovery Workbench, 2013; version LSR-4-0-12).

B Cell Depletion in hFcgRIIb Transgenic Mice

B cell depletion in hFcgRIIb transgenic mice was analyzed using flow cytometry using commercially available antibodies.

### Results and Discussion

As shown in FIG. 6 it is apparent that iv administration of BI-1206 is frequently associated with IRRs, thrombocytopenia, transient spikes in cytokines, and less frequently but in the most severe cases, increases in liver enzymes. As shown in FIG. 7, achieving such sustained high receptor occupancy is required for therapeutic benefit is associated with high levels of IRRs.

### Example 4

In Example 4A and Example 4B, an antibody denoted BI-1206 is used. This antibody has the following light and heavy chains:

Light chain:
(SEQ. ID. No: 1)
QSVLTQPPSASGTPGQRVTISCTGSSSNIGAGYDVHWYQQLPGTAPKLLI
YADDHRPSGVPDRFSGSKSGTSASLAISGLRSEDEADYYCASWDDSQRAV
IFGGGTKLTVLGQPKAAPSVTLFPPSSEELQANKATLVCLISDFYPGAVT
VAWKADSSPVKAGVETTTPSKQSNNKYAASSYLSLTPEQWKSHRSYSCQV
THEGSTVEKTVAPTECS
Heavy chain:
(SEQ. ID. No: 2)
EVQLLESGGGLVQPGGSLRLSCAASGFTFSSYGMHWVRQAPGKGLEWMAV
ISYDGSNKYYADSVKGRFTISRDNSKNTLYLQMNSLRAEDTAVYYCAREL
YDAFDIWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

A modified format of BI-1206 is format wherein the glycosylation site at N297 (marked in bold above) is mutated to a Q (marked in bold below), i.e. an N297Q mutation, resulting in the following heavy chain:

(SEQ. ID. No: 195)
EVQLLESGGGLVQPGGSLRLSCAASGFTFSSYGMHWVRQAPGKGLEWMAV
ISYDGSNKYYADSVKGRFTISRDNSKNTLYLQMNSLRAEDTAVYYCAREL
YDAFDIWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYQSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK

As surrogate antibody and control antibody, the anti-mouse CD32B antibody AT130-2 as IgG2a isotype and the control antibody AT130-2 N297A as IgG1 isotype are used below. AT130-2 as IgG2a isotype is commercially available, for example from ThermoFisher Scientific as Catalog #12-0321-82, however #12-0321-82 is a PE conjugate so the antibody then should be modified so that it is not a conjugate. AT130-2 N297A as IgG1 isotype may be produced by any known method including substituting N in position 297 (as identified above) with A.

### Example 4A—Target: FcvRIIB

### Background

BioInvent International AB has developed the therapeutic monoclonal antibody BI-1206 with anti-tumor activity that can be used as single therapy or in combination with anti-CD20 targeting therapeutics or other clinically validated checkpoints inhibitors. BI-1206 binds with high specificity to CD32B (FcγRIIB) and is currently evaluated in two clinical phase I/IIa studies, CRUKD/16/001 and 17-BI-1206-02, treating patients with chronic lymphocytic leukemia (CLL) and B-cell non-Hodgkin's lymphoma (B-cell NHL). All below described data from the clinical studies is preliminary, only partially quality controlled and should be considered as illustrative of the pharmacodynamic effects and tolerability associated with BI-1206. Part of the data is based on personal communication with individual investigators.

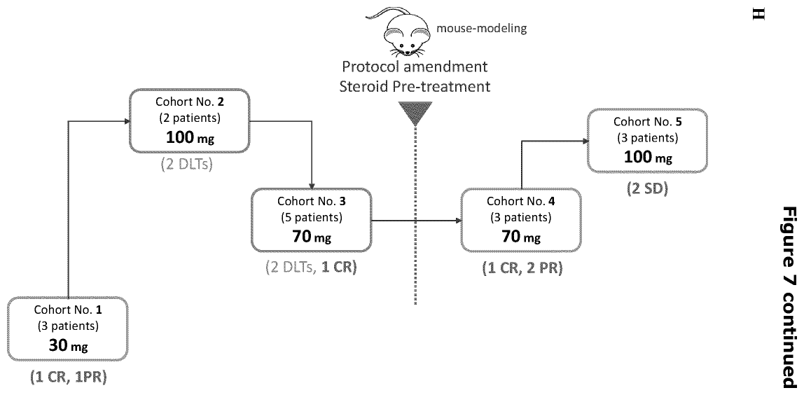

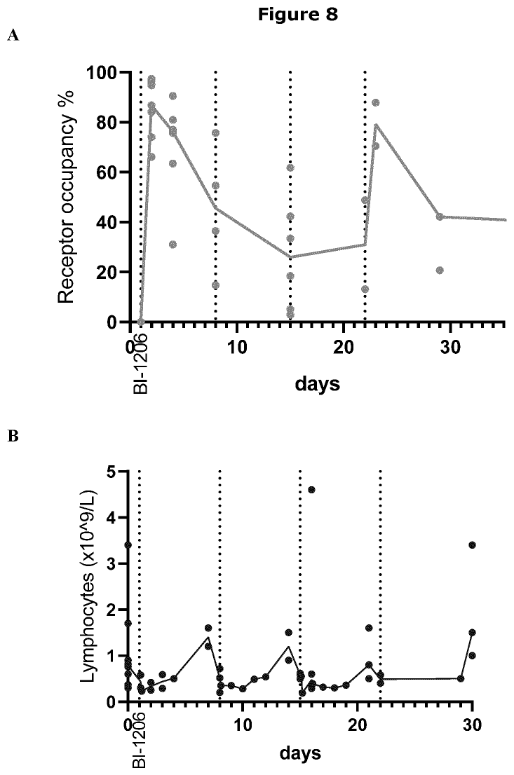

To date up to 100 mg of BI-1206 have been administrated, as monotherapy or in combination with rituximab, to 24 human subjects. 100 mg BI-1206 shows transient receptor saturation on peripheral B cells with 100%, or close to 100%, receptor occupancy for up to 48 hours (FIG. 8). Correspondingly a transient depletion of peripheral B lymphocytes is seen, recovered within approximately 7 days (FIG. 8). This is in line with pre-clinical in vivo models using hFcγRIIB mice where it has been demonstrated that a sustained receptor saturation is necessary to achieve sustained B lymphocyte depletion.

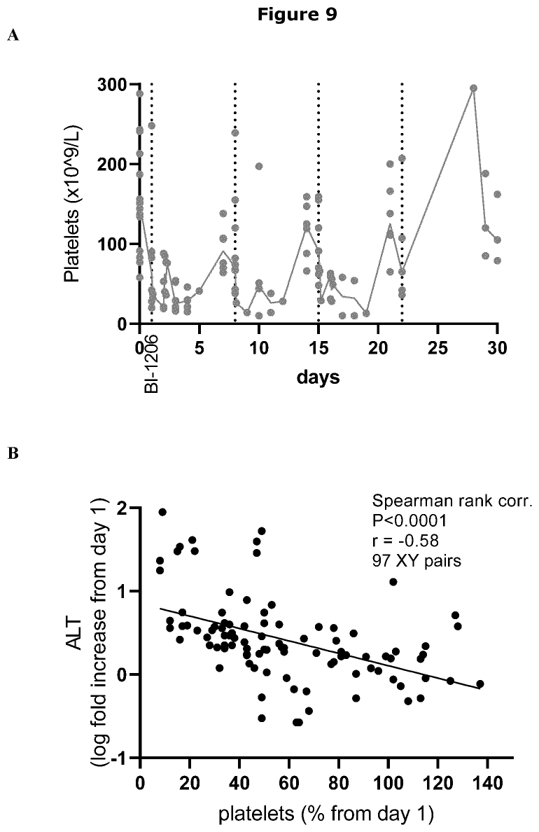

Frequent infusion related reactions (IRRs) have been seen during BI-1206 infusions in human subjects (FIG. 7A). Administration of ≥50 mg BI-1206 is also associated with a transient decrease in platelets (FIG. 9). Thrombocytopenia has not been serious nor associated with bleeding and most episodes resolved within a week. There appears to be a connection between platelet decrease and elevated transaminases (i.e. alanine transaminase (ALT) and aspartate transaminase (AST)), where the ALT and AST increase has been significant in 3 out of 16 subjects receiving ≥70 mg BI-1206 (FIG. 9).

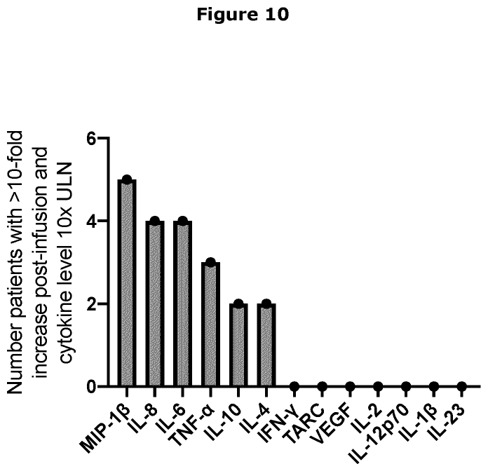

Moreover, a transient cytokine release has been observed in 5 out 5 subjects receiving ≥70 mg BI-1206 where plasma or serum has been available for analysis. The cytokine release includes macrophage inflammatory protein (MIP)-1β, tumor necrosis factor (TNF)-α, interleukin (IL)-10, IL-8, IL-6, and IL-4, and the peak is seen immediately after infusion and cytokines are always normalized within 24 hours (FIG. 10). The cytokine release has not been associated with clinical symptoms.

In the clinical study 17-BI-1206-02, 16 subjects have received 70-100 mg BI-1206, altogether there have been 58 BI-1206 administrations at these dose levels. 46 of these administrations were given following implementation of the in vivo protective corticosteroid-based premedication regimen identified in the animal model, (FIGS. 7C and 7H). Implementation of the in vivo animal model identified premedication regimen into the clinic resulted in statistically significant reduced severity and frequency of IRRs in human cancer patients (FIGS. 7D and 7H).

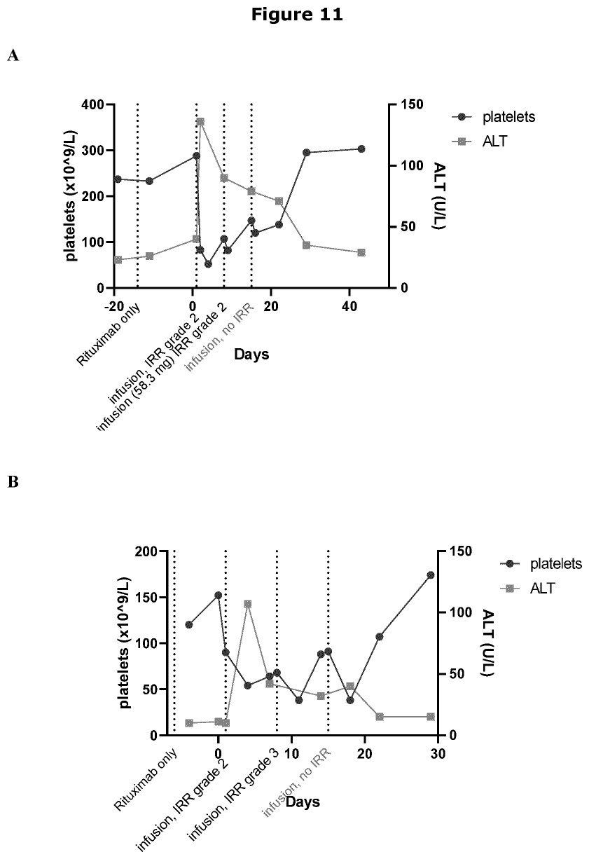

Subjects 501-001 and 503-002 received 12 mg and 4 mg dexamethasone, respectively, the evening before and again 20 mg dexamethasone 30 minutes prior to the third administration of BI-1206 (70 mg) during induction therapy. Both subjects did not suffer IRRs after this premedication regimen using two doses of dexamethasone. During the previous two infusions with BI-1206, where dexamethasone (20 mg) was given only 30 minutes prior to infusion, IRRs (grade 2-3) had been experienced. In addition, in subject 501-001 and 503-002 no/low platelet decrease, and no ALT/AST increase was seen after BI-1206 administration when premedicating with two doses of dexamethasone (FIG. 11). The third subject (201-003) had received 9 administrations of 30 mg of BI-1206 (4 doses during induction phase and 5 doses during maintenance phase) and repeatedly experienced IRR's. At the 10th BI-1206 administration premedication with two doses of dexamethasone was used, and IRR's were improved to grade 1. In subject 201-003 which received a lower dose of BI-1206 (30 mg) platelet decrease or ALT/AST increase had never been seen. Importantly, and consistent with FcgRIIB receptor saturation determining therapeutic efficacy, therapeutic efficacy was maintained following implementation of the premedication regimen in the clinic. Both complete and partial responses have been observed in patients following incorporation of the premedication regimen into the clinical protocol (FIG. 7H).

### Materials & Methods

### Test and Control Substances

The anti-mouse CD32B IgG2a clone AT130-2 and the control antibody (AT130-2 N297A) were transiently expressed in HEK293 cells. The specificity of the purified research batches was demonstrated in a luminescence-based enzyme linked immunosorbent assay (ELISA) or in flow cytometry analyses. Endotoxin-levels of antibodies were found to be <0.1 IU/mL as determined by the LAL-Amoebocyte test.

| Antibody clone | Description |
|---|---|
| AT-130-2 IgG2a | Mouse surrogate of BI-1206 as described above |
| AT-130-2 IgG1 | Fc null version of mouse surrogate of BI-1206 |
| N297A | as described above |

### Mice

Six to eight weeks-old (17-20 g) female C57/BL6 mice were obtained from Taconic. Mice were injected either intra-venous (i.v.), intra-perinatal (i.p.) or sub-cutaneous (s.c.) with mouse anti-CD32B AT-130-2 IgG2a in doses ranging from 1 μg-400 μg/mouse.

### Premedication

For the corticosteroid treatment, Betapred (betamethasone, VNR: 008938, Alfasigma S.P.A.) or Dexamethasone (Cat. No: S1322, batch no: 02, Selleckchem) was used. For the anti-histamine treatment Zyrlex (10 mg/ml, VNR: 523084, MACURE PHARMA ApS), Zantac (25 mg/ml, VNR: 077875, GlaxoSmithKline AB) or Aeurius (0.5 mg/ml, VNR: 097288, Merck Sharp & Dohme BV) was used.

### Animal Monitoring

Mice were monitored post injection with regard to changes in behavior and macroscopic symptoms such as isolation, mobility, and fur condition. Macroscopic IRRs scoring system of 0-2 was set up based on the observations:

| Scoring | Macroscopic symptoms |
|---|---|
| 0 | No visible symptoms |
| 1 | Isolation, decreased activity |
| 2 | Isolation, decreased activity, impaired balance, piloerection, |
| | hunching followed by un-natural body posture |

### Blood Sampling

Blood samples were collected from vena saphena for instant blood count analysis. For serum concentrations of AT130-2, liver enzyme and cytokine analysis, the mice were bled from the aorta under isoflurane anesthesia just prior to sacrifice.

### Serum Concentrations of AT130-2

Serum concentrations of AT130-2 mAb has been was quantified using a sandwich ELISA. Briefly, recombinant CD32B protein (Sino Biological #50030-M08H) was used as coating. Diluted samples were added to the ELISA plate, and following incubation and washing steps, detection was conducted via an HRP conjugated polyclonal donkey-anti-mouse-IgG Ab (Jackson #715-035-151). Subsequently the Pico Chemiluminescent Substrate (ThermoFisher #37069) were used and plate reading was performed with a Tecan Ultra Microplate reader.

### Platelet Count

Platelet counts were analyzed in fresh blood using a Vetscan (Vetscan HM5 Abaxis, Triolab).

### Transaminases

Transaminases were analyzed shipping of frozen serum samples to (IDEXX BioResearch Vet Med Labor GmbH).

### Cytokines

To study the potential contributors of infusion-related reactions (IRRs) in the mice, cytokine release has been evaluated at selected timepoints in association with i.p. injection of AT-130-2 mAb. Serum samples frozen once were thawed and diluted ×2 or ×4. Cytokines were analyzed with the V-plex Proinflammatory Panel 1 Mouse kit (MesoScale Discovery #K15048D), including the analytes interferon (IFN)-γ, interleukin (IL)-1β, IL-2, IL-4, IL-5, IL-6, IL-10, IL-12p70, KC/GRO, tumor necrosis factor (TNF)-α. The assay followed the manufacturer's protocol as outlined briefly: 50 μL of sample and calibration standard were added to the MSD plates and incubated. Following washing, 25 μL of SULFO-TAG detection antibody mixture were added to each well of the corresponding plate. The plates were analyzed on a QuickPlex SQ120 Reader instrument (MSD) and cytokine concentration was calculated using the MSD software (Discovery Workbench, 2013; version LSR-4-0-12).

### Results

Macroscopic Symptoms after Murine Surrogate Anti-CD32b IgG2a (AT-130-2)

The murine surrogate anti-CD32b (AT-130-2 IgG2a) was injected into wildtype C57/BL6 mice through 3 different injection routes, intravenously (i.v.), intraperitoneally (i.p.) or subcutaneously (s.c.). At 200 μg (corresponding to 10 mg/kg) a rapid onset of infusion-related reactions (IRRs) was seen 5-7 minutes after i.v. injection. These IRRs included isolation, decreased activity, impaired balance, piloerection, hunching followed by un-natural body posture. Blood sampling of these mice indicated reduced blood pressure. 10-15 minutes post IRRs onset these mice started to recover and 1 h post injection no macroscopic symptoms were seen.

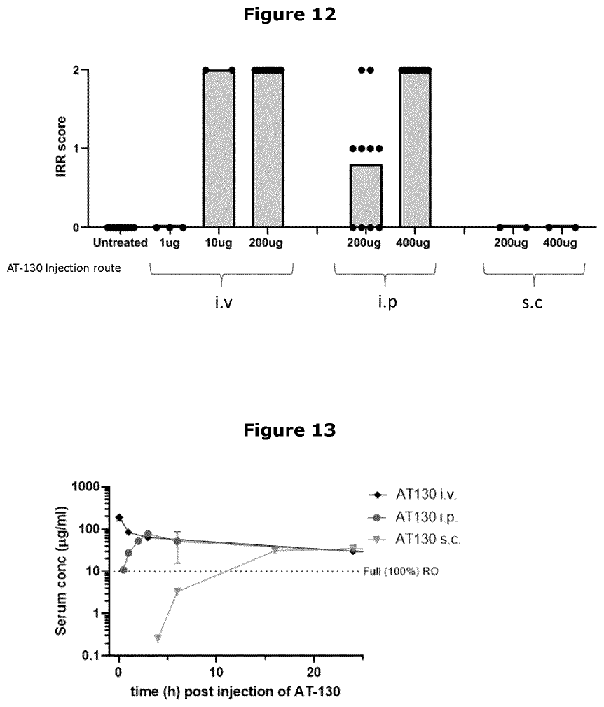

When titrating the i.v. dose, the same timing and severity of macroscopic symptoms was seen down to 10 μg (0.5 mg/kg). However, at 1 μg (0.05 mg/kg) no IRRs were seen (FIG. 12).

When administrating the same dose 200 μg (10 mg/kg) i.p. a delay in IRRs onset was seen with the IRRs appearing 20-30 minutes post injection. In contrast to the i.v. injection route all mice in this group did not display IRRs and the IRRs were less severe in several mice (FIG. 12).

When increasing the i.p. dose to 400 μg (20 mg/kg) the onset of IRRs was still delayed compared to the i.v. injection route however, all mice displayed IRRs to the same extent and grade as 200 μg i.v. (FIG. 12). All mice had fully recovered 1 h post injection.

Finally, when administrating 200 μg to mice s.c. no IRRs were seen (up to 24 h post injection). When increasing the s.c. dose to 400 μg the mice remained unaffected (FIG. 12).

When administrating the Fc-null version of AT-130-2, AT-130-2 IgG1 N297A i.v. no IRRs were seen, indicating that Fc-binding is necessary to incite the symptoms associated with AT-130-2.

The pharmacokinetic profiles of AT-130-2 was assessed for i.v., i.p., and s.c. injection (FIG. 13).

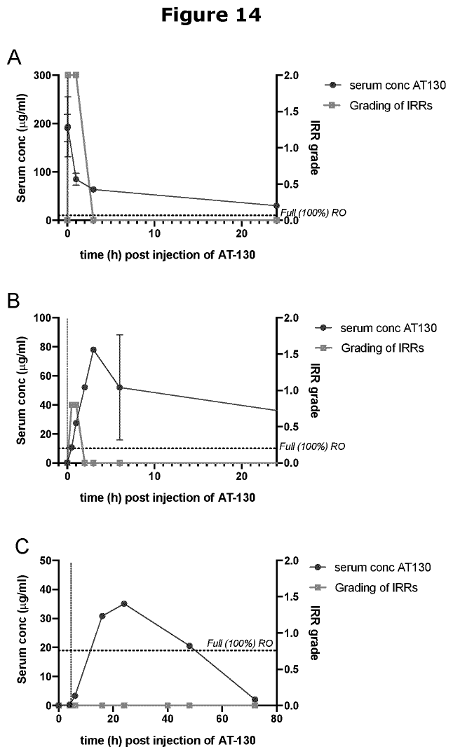

When comparing the PK and the presumed receptor occupancy (RO, based on separate experiments not shown here where it was shown that 10 μg/ml gives 100% receptor saturation) with the onset, severity and duration of IRRs it is clear that there is a correlation between high and rapid exposure of AT-130-2, rather than time of FcγRIIB saturation. Tolerability showing a clear pattern of s.c. >i.p. >i.v. with RO being sustained for a long period of time post IRRs recovery (FIG. 14).

### Platelets, Transaminases and Cytokines

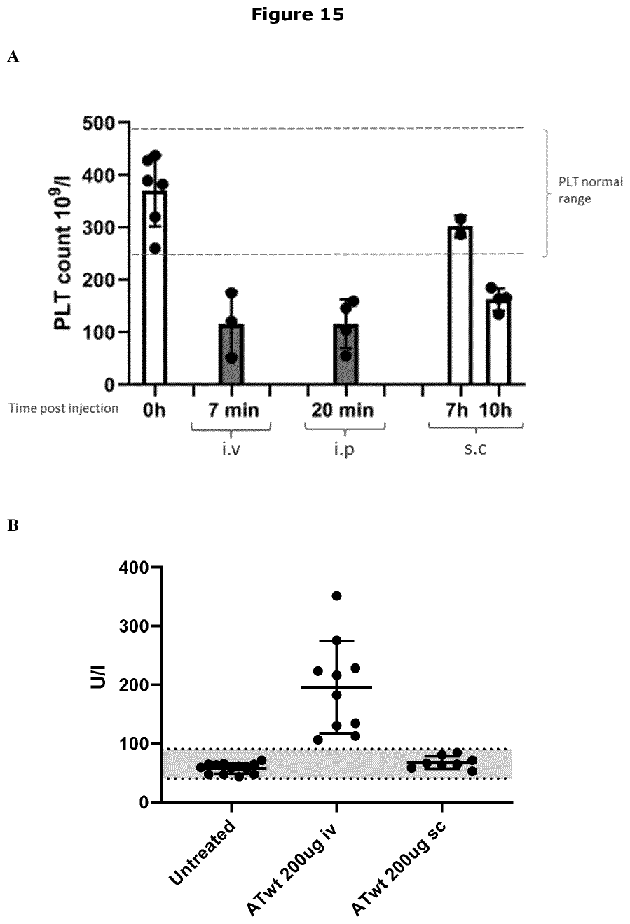

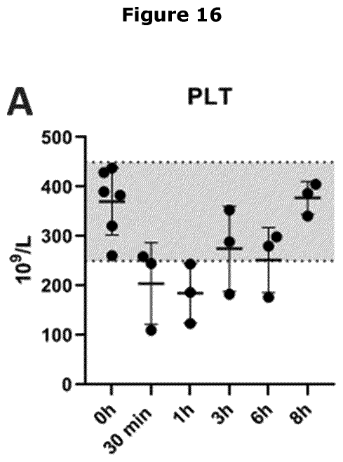

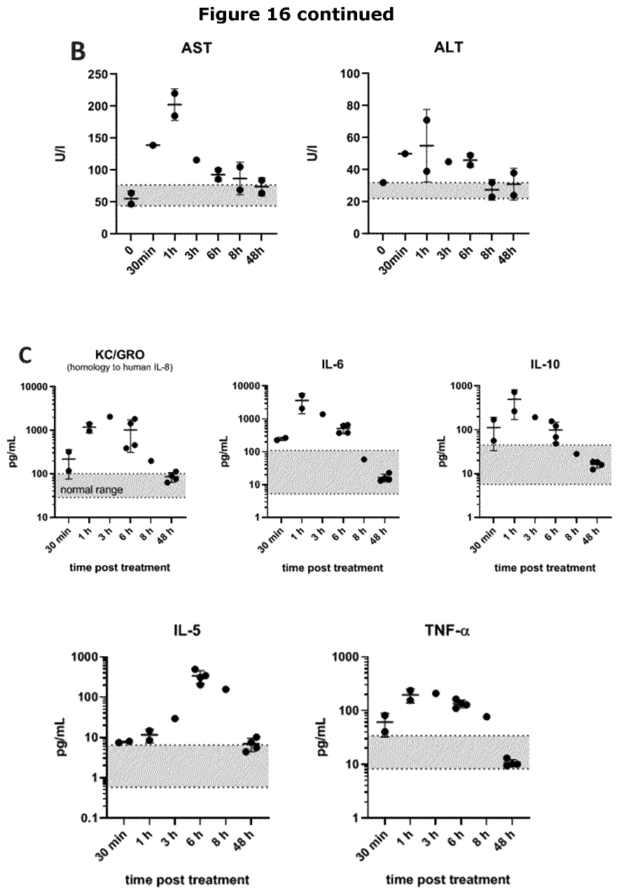

To investigate if the IRRs seen in these mice were associated with other parameters seen in the clinical studies with BI-1206 mice were bled at the onset of IRRs and the blood was analyzed for blood cell count, clinical chemistry parameters and cytokines. In the case of s.c. injection where no IRRs occurred, mice were bled at different timepoints post injection. A decrease in platelet count (PLT) was seen at the same time as IRRs onset after injection of AT-130-2 through both the i.v. and i.p. administration route (FIG. 15A). For the s.c. administration route only a moderate drop was seen 10 h post injection (FIG. 15A). In all cases the PLT decrease was transient and was restored to values in the normal range within 8 h post injection (data for 200 μg AT-130-2 injected i.p. is shown in FIG. 16A). When administering AT-130-2 IgG2a s.c. the transaminase increase seen following i.v. injection is circumvented. For the i.v. administration route an increase is seen in transaminases (AST) 1 h post onset of macroscopic symptoms (previously established as time point for a peak value in transaminases). However, for the s.c. administration route no macroscopic symptoms were seen. More specifically, no increase in transaminases was seen 11 hours post injection (1 h post the time of FcγRIIB saturation according to PK (10 h)), as shown in FIG. 15B.

With regard to clinical chemistry parameters an increase in transaminases (AST and ALT) with a peak 1 h post injection was the only parameter affected by AT-130-2 injection. These increases were just like the PLT decrease transient (FIG. 16B). The same transient increase was seen when AT-130-2 was injected i.v. and no increase in transaminases was detected when AT-130-2 was injected s.c.

A panel of cytokines including the analytes IFN-γ, IL-1β, IL-2, IL-4, IL-5, IL-6, IL-10, IL-12p70, KC/GRO, TNF-α were analyzed at different time points post injection of 200 μg i.p. Of all the analyzed cytokines IL-5, IL-6, IL-10, KC/GRO, TNF-α showed a transient increase, all peaking 1-3 hours post injection, except for IL-5 (FIG. 16C). IL-5 showed a delayed peak 3-8 hours post injection (FIG. 16C). These were the same cytokines that have been indicated to increase in some patients in the clinical studies with BI-1206.

### Premedication

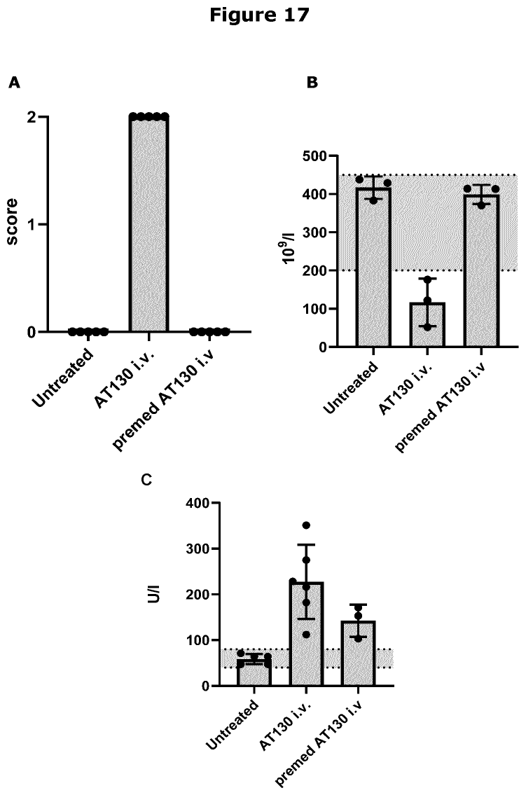

In order to investigate if premedication with corticosteroids could inhibit the IRRs and associated toxicities of AT-130-2, mice were premedicated with 40 mg/kg betamethasone 16-24 h and 1 h pre injection of AT-130-2. Both the IRRs and the platelet decrease seen with AT-130-2 was completely inhibited with premedication (FIG. 17A-B). Also, the increase in liver transaminases and cytokine release was less profound (FIG. 17C and data not shown). The same effect was seen when another corticosteroid, dexamethasone was assessed (data not shown).

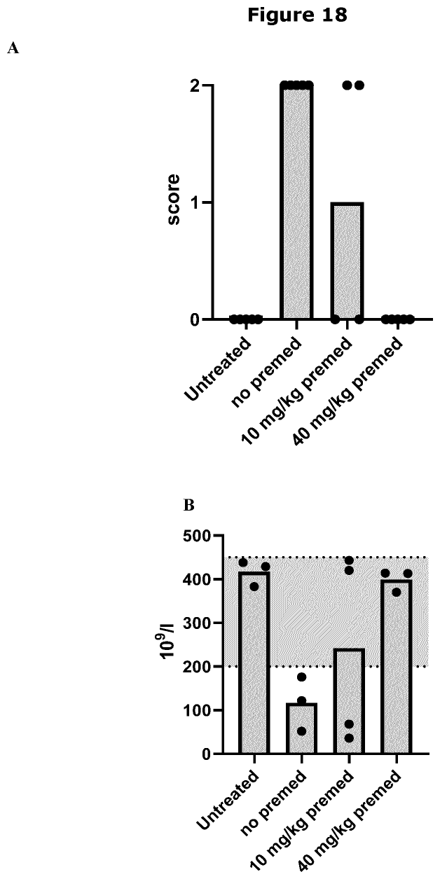

To assess the importance of the dose of corticosteroid treatment the dose of betamethasone was decreased from 40 mg/kg to 10 mg/kg in the following experiment (FIG. 18). At 10 mg/kg both IRRs and a decrease in platelet count were seen in half of the mice indicating that a high dose of corticosteroids is needed to completely block the IRRs and associated toxicities (FIG. 18).

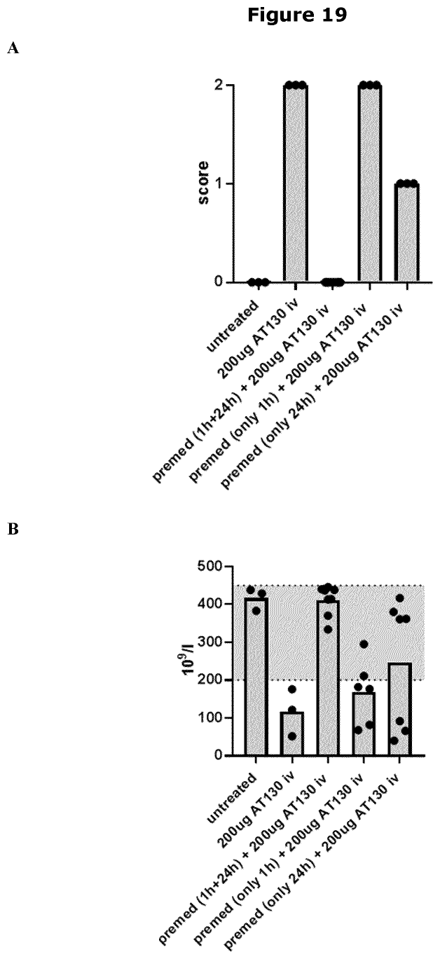

Further, the importance of the two doses of corticosteroid treatment was investigated by comparing the protective effect of only early (1 hours pre injection) or only late (24 hours pre injection) premedication with the two doses of corticosteroid treatment. Premedication only 1 hour pre injection could not inhibit the IRRs nor the decrease in platelet count (FIG. 19). Premedication 24 h pre injection diminished the IRRs and the platelet decrease but could not completely block these symptoms (FIG. 19), indicating that two doses of corticosteroids is needed to fully block IRRs.

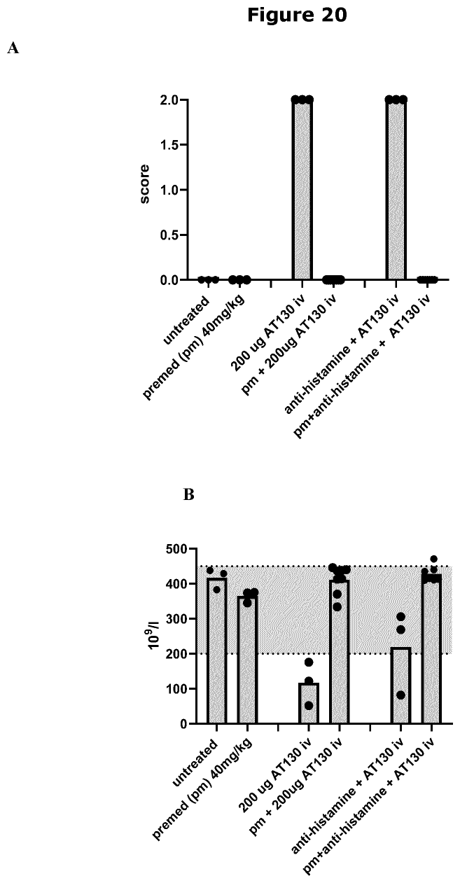

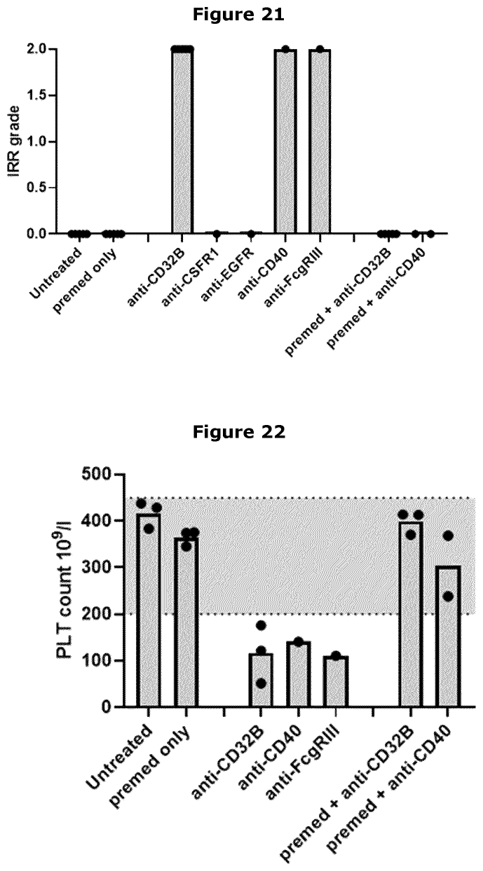

Finally, the impact of antihistamine, which is a standard premedication in the clinical trials, was evaluated. Premedication with antihistamine alone did not inhibit IRRs or platelet decrease. When combining the two doses of corticosteroid treatment with antihistamine pretreatment the protective effect was retained (FIG. 20). These results were confirmed for three different types of antihistamines (Zyrlex, Zantac and Aeurius).

### Conclusions

Our data demonstrates an in vivo model using intra-venous (i.v.) or intra-perinatal (i.p.) administration of anti-FcγRIIB mIgG2a surrogate (AT-130-2) in wild type mice recapitulates the tolerability profile seen with BI-1206, including IRR's, decreased platelet count, elevated transaminases (i.e. ALT and AST) and transient cytokine release. The IRR's appear 5-20 minutes after AT-130-2 injection with macroscopic symptoms including isolation, decreased activity, impaired balance, piloerection, hunching followed by un-natural body posture and decreased blood pressure. The visual physical reaction is transient, and the animals are fully recovered 1 h after the antibody administration. The macroscopic symptoms are accompanied by a decrease in platelet count and elevated transaminases which are normalized within 8 hours. The cytokine release is acute and transient and includes IL-6, IL-5, IL-10, TNFα and KC/GRO (rodent homolog of human IL-8). The cytokine profile and kinetics is equivalent to what is seen after BI-1206 in human subjects. In the mouse model there is an apparent correlation between the IRR's and high and rapid exposure, rather than time of FcγRIIB saturation, where sub-cutaneous (s.c.) administration of AT-130-2 is better tolerated than i.p. and i.v. administration. The timing of onset of symptoms correlates with the serum concentration where receptor saturation is achieved. However, also when administrating an antibody dose that achieves receptor saturation for 6 days or longer the animals recover from all symptoms within 24 h. Sustained FcγRIIB blockade per se does not appear to be the causative of IRRs.

In this model premedication with two doses of corticosteroids (dexamethasone or betamethasone) inhibits the macroscopic IRRs as well as platelet decrease and transaminase elevation. The two doses are given s.c. 16-24 hours and i.v. 30-60 minutes prior to antibody administration. The prevention of macroscopic symptoms in the mice by corticosteroids is dose-dependent and importantly the timing of premedication is crucial. The dose 16-24 hours prior to antibody administration is imperative in order to gain the protective effect. If corticosteroids are only given 30-60 minutes prior to antibody administration no protective effect with regard to the macroscopic symptoms is seen, whereas the dose 16-24 hours prior to antibody administration alone partially improves tolerability. When both doses are given, inhibition of the macroscopic symptoms, platelet decrease, transaminase elevation, and cytokine release is achieved.

Dosing of human patients according to the corticosteroid-based regimen identified in the mouse model protected against IRRs and allowed for administration of higher doses, which are likely to be associated with stronger antitumor activity, of the studied anti-FcgRIIB antibody.

### Example 4B—Other Targets

### Materials & Methods

### Test and Control Substances

The anti-mouse CD32b clone was transiently expressed in HEK293 cells. The specificity of the batch was demonstrated in a luminescence-based enzyme linked immunosorbent assay (ELISA) or in flow cytometry analyses. Endotoxin-levels of antibodies were found to be <0.1 IU/mL as determined by the LAL-Amoebocyte test. The anti-mouse CD40, EGFR and CSFR1 antibodies were purchased from BioXcell or Absolute Antibody (see table below) and the anti-mouse FcγRIII antibody AT154-2 was a gift from University of Southampton. Alternatively, AT154-2 as rat IgG2b isotype may be purchased from, for example, BioRad, Argio Biolaboratories (ARG23942) or LSBio (LS-C745656) which is then converted into IgG2a format using any well-known method.

| Antibody clone | Description | Reference |
|---|---|---|
| AT-130-2 Mouse | anti-mouse CD32b | (See comments above |
| IgG2a | | Example 1) |
| FGK4.5/FGK45 | anti-mouse CD40 | BP0016-2, BioXcell, |
| Rat IgG2a | | |
| 7A7 Mouse IgG2a | anti-mouse EGFR | Ab00134-2.0, Absolute |
| | | Antibody |
| AFS98 Rat IgG2a | anti-mouse CSFR1 | BE0213, BioXcell |
| AT154-2 Mouse | anti-mouse FcγRIII | (See comments above |
| IgG2a | | the table) |

### Mice

Six to eight weeks-old (17-20 g) female C57/BL6 mice were obtained from Taconic. Mice were injected intra-venous (i.v.), with 200 μg/mouse of the different antibodies.

### Premedication

For the corticosteroid treatment, Betapred (betamethasone, VNR: 008938, Alfasigma S.P.A.) or Dexamethasone (Cat. No: S1322, Batch No: 02, Selleckchem) was used.

### Animal Monitoring

Mice were monitored post injection with regard to changes in behavior and macroscopic symptoms such as isolation, mobility, and fur condition. Macroscopic IRRs scoring system of 0-2 was set up based on the observations:

| Scoring | Macroscopic symptoms |
|---|---|
| 0 | No visible symptoms |
| 1 | Isolation, decreased activity |
| 2 | Isolation, decreased activity, impaired balance, piloerection, |
| | hunching followed by un-natural body posture |

### Blood Sampling

Blood samples were collected from vena saphena for instant blood count analysis.

### Platelet Count

Platelet counts were analyzed in fresh blood using a Vetscan (Vetscan HM5 Abaxis, Triolab).

### Conclusions

This example shows that the model described herein can distinguish between antibody molecules that induce tolerability issues and those that do not. It further shows that premedication can inhibit IRRs related to different antibodies and targets.

This example also shows that antibodies that induce IRRs also induce thrombocytopenia. Furthermore, it demonstrates that premedication can inhibit thrombocytopenia related to different antibodies and targets.

### EMBODIMENTS OF THE INVENTION

Certain embodiments of the invention will be described with reference to the following numbered paragraphs:
1. A therapeutic system for use in improving tolerability of an antibody molecule that specifically binds to FcγRIIb in a subject, wherein the therapeutic system comprises:
   (i) an antibody molecule that specifically binds to FcγRIIb, wherein the antibody molecule is administered to the subject as at least a first dose and a second dose; and
   (ii) a corticosteroid, wherein the first dose of the antibody molecule is lower than the maximum therapeutically effective dose of the antibody molecule; and wherein the corticosteroid is administered to the subject before the first dose of the antibody molecule.
2. A combination comprising an antibody molecule and a corticosteroid for use in a dosage regimen for improving tolerability of an antibody molecule that specifically binds to FcγRIIb in a subject, wherein the dosage regimen comprises the following steps:
   (i) administration of a corticosteroid before administration of a first dose of the antibody molecule;
   (ii) administration of the first dose of the antibody molecule that specifically binds to FcγRIIb that is lower than the maximum therapeutically effective dose; and
   (iii) administration of a second dose of the antibody molecule that specifically binds to FcγRIIb, wherein the first dose of the antibody molecule is administered prior to the second dose.
3. Use of:
   (i) an antibody molecule that specifically binds to FcγRIIb; and
   (ii) a corticosteroid, in the manufacture of a medicament for improving tolerability of an antibody molecule that specifically binds to FcγRIIb in a subject, wherein the medicament comprises at least a first dose and a second dose of the antibody molecule; and wherein the first dose of the antibody molecule is lower than the maximum therapeutically effective dose of the antibody molecule; and wherein the corticosteroid is administered before the first dose of the antibody molecule.
4. A method for improving tolerability of an antibody molecule that specifically binds to FcγRIIb in a subject comprising:
   (i) administration of a corticosteroid before administration of a first dose of the antibody molecule;
   (ii) administration of the first dose of the antibody molecule that specifically binds to FcγRIIb that is lower than the maximum therapeutically effective dose; and
   (iii) administration of a second dose of the antibody molecule that specifically binds to FcγRIIb, wherein the first dose of the antibody molecule is administered prior to the second dose.
5. The system, combination for use, use, or method of paragraphs 1-4, wherein the system, combination for use, use, or method further comprises administration of one or more therapeutic antibodies for the treatment of cancer in a subject.
6. The system, combination for use, use, or method of paragraph 5, wherein the therapeutic antibody is selected from: rituximab; pembrolizumab; nivolumab; cemiplimab; camrelizumab; dostarlimab; obinutuzumab; ofatumumab, and biosimilars or equivalents thereof.
7. The system, combination for use, use, or method of paragraphs 1-6, wherein the corticosteroid is administered to the subject at a time point from 10 minutes to 48 hours before the first dose of the antibody molecule that specifically binds to FcγRIIb.
8. The system, combination for use, use, or method of paragraph 7, wherein the corticosteroid is administered to the subject at a time point from 10 minutes to 24 hours before the first dose of the antibody molecule that specifically binds to FcγRIIb.
9. The system, combination for use, use, or method of paragraphs 1-6, wherein the corticosteroid is administered as a first dose and a second dose, and wherein the first dose of the corticosteroid is administered at a time point from 16 hours to 48 hours before the first dose of the antibody molecule that specifically binds to FcγRIIb, and wherein the second dose of the corticosteroid is administered at a time point from 10 minutes to 2 hours before the first dose of the antibody molecule that binds specifically to FcγRIIb.
10. The system, combination for use, use, or method of paragraph 9, wherein a further dose of corticosteroid is administered at a time point from 16 hours to 48 hours before the second dose of the antibody molecule that specifically binds to FcγRIIb.
11. The system, combination for use, use, or method of paragraphs 1-10, wherein the first dose of the antibody molecule that specifically binds to FcγRIIb is administered at a time point from one to 24 hours before the second dose of the antibody molecule that specifically binds to FcγRIIb.
12. The system, combination for use, use, or method of paragraphs 1-10, wherein the first dose of the antibody molecule that specifically binds to FcγRIIb is administered about one hour before the second dose of the antibody molecule that specifically binds to FcγRIIb.
13. The system, combination for use, use, or method of paragraphs 1-10, wherein the first dose of the antibody molecule that specifically binds to FcγRIIb is administered about 24 hours before the second dose of the antibody molecule that specifically binds to FcγRIIb.
14. The system, combination for use, use, or method of paragraphs 1-10, wherein the first dose of the antibody molecule that specifically binds to FcγRIIb is administered from 24 hours to 48 hours before the second dose of the antibody molecule that specifically binds to FcγRIIb.
15. The system, combination for use, use, or method of paragraphs 1-14, wherein the corticosteroid is administered at a dose of 4 mg or greater.
16. The system, combination for use, use, or method of paragraphs 1-15, wherein the corticosteroid is administered at a dose of 12 mg or greater.
17. The system, combination for use, use, or method of paragraphs 1-14, wherein the corticosteroid is administered at a dose of from 4 mg to 20 mg.
18. The system, combination for use, use, or method of paragraph 17, wherein the corticosteroid is administered at a dose of from 12 mg to 20 mg.
19. The system, combination for use, use, or method of paragraph 17, wherein the corticosteroid is administered at a dose of from 4 mg to 12 mg.
20. The system, combination for use, use, or method of paragraphs 1-19, wherein the corticosteroid is dexamethasone or betamethasone or a combination of dexamethasone and betamethasone.
21. The system, combination for use, use, or method of paragraphs 1-20, wherein the first dose of the antibody molecule that specifically binds to FcγRIIb is lower than the maximum tolerated therapeutic dose.
22. The system, combination for use, use, or method of paragraphs 1-21, wherein the first dose of the antibody molecule that specifically binds to FcγRIIb is at least 50% lower than the maximum therapeutically effective dose.
23. The system, combination for use, use, or method of paragraphs 1-22, wherein the first dose of the antibody molecule that specifically binds to FcγRIIb is administered at a dose of from 0.2 mg/kg to 0.6 mg/kg.
24. The system, combination for use, use, or method of paragraph 23, wherein the first dose of the antibody molecule that specifically binds to FcγRIIb is administered at a dose of from 0.3 mg/kg to 0.5 mg/kg.
25. The system, combination for use, use, or method of paragraphs 1-24, wherein the first dose of the antibody molecule that specifically binds to FcγRIIb is administered at a dose of from 20 mg to 40 mg.
26. The system, combination for use, use, or method of paragraph 25, wherein the first dose of the antibody molecule that specifically binds to FcγRIIb is administered at a dose of about 30 mg.
27. The system, combination for use, use, or method of paragraphs 1-26, wherein the second dose of the antibody molecule that specifically binds to FcγRIIb is a therapeutically effective dose.
28. The system, combination for use, use, or method of paragraphs 1-27, wherein the second dose of the antibody molecule that specifically binds to FcγRIIb is the maximum tolerated therapeutic dose or the maximum feasible therapeutic dose.
29. The system, combination for use, use, or method of paragraphs 1-27, wherein the second dose of the antibody molecule that specifically binds to FcγRIIb is lower than a therapeutically effective dose.
30. The system, combination for use, use, or method of paragraphs 1-29, wherein further additional doses of the antibody molecule that specifically binds to FcγRIIb are administered to the subject following the second dose of the antibody molecule that specifically binds to FcγRIIb.
31. The system, combination for use, use, or method of paragraphs 1-30, wherein infusion related reactions associated with the administration of the antibody molecule that specifically binds to FcγRIIb are reduced or eliminated.
32. The system, combination for use, use, or method of paragraphs 1-31, wherein changes to the body temperature and/or platelet count and/or blood levels of liver enzymes of the subject are reduced (and are preferably reduced to acceptable levels) for at least 24 hours following administration of the second dose of the antibody molecule that specifically binds to FcγRIIb.
33. The system, combination for use, use, or method of paragraphs 1-32, wherein the antibody molecule that specifically binds to FcγRIIb is capable of binding one or more Fcγ receptors via its Fc region.
34. The system, combination for use, use, or method of paragraphs 1-33, wherein the antibody molecule that specifically binds to FcγRIIb has a light chain sequence of SEQ ID No: 1 and a heavy chain of SEQ ID No: 2.
35. The system, combination for use, use, or method of paragraphs 1-34, wherein the first and second doses of the antibody molecule that specifically binds to FcγRIIb are formulated for intravenous delivery to the subject.
36. The system, combination for use, use, or method of paragraphs 1-35, wherein the corticosteroid is formulated for intravenous or oral delivery to the subject.
37. A kit comprising:
   (i) an antibody molecule that specifically binds to FcγRIIb, optionally as defined in paragraphs 33 and/or 34;
   (ii) a corticosteroid, optionally as defined in any one of paragraphs 15-20; and
   (iii) optionally, instructions for use, wherein the antibody molecule is provided as a first dose and a second dose, wherein the first dose of the antibody molecule is lower than the maximum therapeutically effective dose of the antibody molecule, further optionally wherein the first dose is as defined in paragraphs 11-14 and 21-26, further optionally wherein the second dose is as defined in paragraphs 27-29.
38. The kit of paragraph 37, wherein the kit is for improving the tolerability of the antibody molecule in a subject.
39. The kit of paragraph 37 or 38, wherein the corticosteroid is provided in a dose as defined in any one of paragraphs 12-17.
40. The kit of paragraphs 37-39, wherein the kit further comprises one or more therapeutic antibodies.
41. The kit of paragraph 40, wherein the therapeutic antibody is selected from: rituximab; pembrolizumab; nivolumab; cemiplimab; camrelizumab; dostarlimab; obinutuzumab; ofatumumab, and biosimilars or equivalents thereof.
42. The kit of paragraph 40 or 41, wherein the kit is for use in treating cancer in a subject.
43. The system, combination for use, use, method, or kit substantially as described herein with reference to the description and drawings.
44. A method for predicting if a therapeutic antibody molecule binding specifically to a human target will be associated with a tolerability issue in connection with intravenous administration to a human, comprising the following step:
   (i) intravenous or intraperitoneal administration of the therapeutic antibody molecule, if cross-reactive with murine target, or a surrogate antibody, to a mouse and observation of the mouse during a period following immediately after the administration of the therapeutic or surrogate antibody, wherein a display of the macroscopic symptoms isolation and decreased activity during the period followed by restoration of the state of the mouse to the normal state is an indication that the intravenous administration of the therapeutic antibody molecule to a human will be associated with a tolerability issue, and/or for predicting if a prophylactic or therapeutic treatment, an altered administration route and/or a modification of the therapeutic antibody molecule can prevent or mitigate a tolerability issue associated with intravenous administration to a human of a therapeutic antibody molecule binding specifically to a human target, comprising the following step(s) in addition to (i) as set out above:
   (ii) administration of a prophylactic or therapeutic agent to a mouse in conjunction with intravenous or intraperitoneal administration of the therapeutic or surrogate antibody to a mouse, and observation of the mouse during a period following immediately after the administration of the therapeutic or surrogate antibody, wherein a decreased display of the macroscopic symptoms compared to the macroscopic symptoms displayed by the mouse in (i) or no display of the macroscopic symptoms during the period is an indication that pre-treatment with the prophylactic or therapeutic agent in combination with administration of the therapeutic antibody molecule to a human can prevent or mitigate the tolerability issue that otherwise would be associated with intravenous administration of the therapeutic antibody molecule to a human;
   (iii) administration of the therapeutic or surrogate antibody to a mouse by a route of administration other than intravenous or intraperitoneal administration, and observation of the mouse during a period following immediately after the administration of the therapeutic or surrogate antibody, wherein a decreased display of the macroscopic symptoms compared to the macroscopic symptoms displayed by the mouse in (i) or no display of the macroscopic symptoms during the period is an indication that the other route of administration can be used for administration of the therapeutic antibody molecule to a human to prevent or mitigate the tolerability issue that would be associated with intravenous administration of the therapeutic antibody molecule to a human; and/or
   (iv) intravenous or intraperitoneal administration of a modified format of the therapeutic or surrogate antibody to a mouse by a route of administration other than intravenous or intraperitoneal administration, and observation of the mouse during a period following immediately after the administration of the modified therapeutic or surrogate antibody, wherein a decreased display of the macroscopic symptoms compared to the macroscopic symptoms displayed by the mouse in (i) or no display of the macroscopic symptoms during the period is an indication that administration of the therapeutic antibody molecule in the modified format to a human can be used to prevent or mitigate the tolerability issue that would be associated with intravenous administration of the therapeutic antibody molecule to a human.
45. A method according to paragraph 44, wherein a display in (i) of 1-3 additional macroscopic symptoms selected from impaired balance, piloerection, and hunching followed by un-natural body posture during the period in (i) where after the state of the mouse is restored to the normal state further strengthens the indication that the intravenous administration of the therapeutic antibody molecule to the human will be associated with a tolerability issue.
46. A method according to paragraph 44 or 45, wherein the period during which the macroscopic symptoms are displayed in (i) starts 5-10 minutes after administration of the therapeutic or surrogate antibody and ends 45-90 minutes after administration of the therapeutic or surrogate antibody, and wherein the observation period in (ii), (iii) and/or (iv) is of the same length.
47. A method according to any one of the paragraphs 44-46, wherein at least one of the following additional parameters:
   decreased blood pressure
   decreased platelet count, and or
   increased liver enzymes (AST/ALT).
   observed during the period in (i) further strengthens the indication that the intravenous administration of the therapeutic antibody molecule to a human will be associated with a tolerability issue.
48. A method for predicting if a prophylactic or therapeutic treatment can prevent or mitigate a tolerability issue associated with intravenous administration to a human of a therapeutic antibody molecule binding specifically to a human target according to any one of the claims 1-4 comprising at least steps (i) and (ii), wherein pre-treatment is used in (ii) and wherein this pre-treatment is administration of a corticosteroid to the mouse prior to injection of the therapeutic or surrogate antibody.
49. A method according to paragraph 48, wherein the pre-treatment comprises two administrations of a corticosteroid, wherein one is given 10⁻⁴⁸ hours prior to administration of the therapeutic or surrogate antibody and the other is given 5 minutes-5 hours prior to administration of the therapeutic or surrogate antibody.
50. A method according to paragraph 49, wherein the corticosteroid is dexamethasone or betamethasone.
51. A method for predicting if an altered administration route can prevent or mitigate a tolerability issue associated with intravenous administration to a human of a therapeutic antibody molecule binding specifically to a human target according to any one of the paragraphs 44-50 comprising at least steps (i) and (iii), wherein the route of administration used in (iii) is subcutaneous administration.
52. A method for predicting if a modification of the therapeutic antibody molecule can prevent or mitigate a tolerability issue associated with intravenous administration to a human of a therapeutic antibody molecule binding specifically to a human target according to any one of the paragraphs 44-51 comprising at least steps (i) and (iv), wherein the modified format of the therapeutic or surrogate antibody used in (iv) is a modification that leads to decreased or abolished engagement of Fc receptors.
53. A method according to any one of the paragraphs 44-52, wherein the human target is selected from the group consisting of FcγRIIB, FcγRIIA and CD40.
54. A method according to paragraph 53, wherein the therapeutic antibody molecule is a human anti-FcγRIIB antibody capable of binding a human FcγR via its Fc domain and wherein the mouse surrogate antibody is an anti-FcγRIIb antibody capable of binding a mouse FcγR via its Fc domain.
55. A method according to paragraph 54, wherein the therapeutic antibody molecule is a human anti-FcγRIIB IgG1 antibody and wherein the mouse surrogate antibody is an anti-FcγRIIb mIgG2a.
56. A corticosteroid for use in a dosing regimen to prevent or mitigate a tolerability issue in connection with intravenous administration of a therapeutic antibody molecule to a subject,
   wherein the therapeutic antibody molecule has been predicted to be associated with a tolerability issue in connection with intravenous administration to a human using the method of any one of the paragraphs 44-56, and/or wherein pre-treatment with the corticosteroid combination with administration of the therapeutic antibody molecule to a human has been predicted to prevent or mitigate the tolerability issue that otherwise would be associated with intravenous administration of the therapeutic antibody molecule to a human using the method of any one of the paragraphs 48-50, or any one of the paragraphs 53-55 when referring to any one of the paragraphs 48-50,
   and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).
57. A corticosteroid for use in a dosing regimen to prevent or mitigate a tolerability issue in connection with intravenous administration of a therapeutic antibody molecule to a subject,
   wherein the therapeutic antibody molecule is an anti-FcγRIIB antibody,
   and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).
58. A corticosteroid for use according to paragraph 56 or 57, wherein the first dose is given 6-36 hours prior to start of administration of the therapeutic antibody molecule and the second dose is given 15-120 minutes prior to start of administration of the therapeutic antibody molecule.
59. A corticosteroid for use according to paragraph 56-58, wherein the first dose is given 16-24 hours prior to start of administration of the therapeutic antibody molecule.
60. A corticosteroid for use according to any one of the paragraphs 56-59, wherein the second dose is given 30-60 minutes prior to start of administration of the therapeutic antibody molecule.
61. A corticosteroid for use according to any one of the paragraphs 56-60, wherein the dosing regimen comprises administration of the at least two doses of the corticosteroid prior to each infusion of the antibody during the course of antibody therapy.
62. A corticosteroid for use according to claim any one of the paragraphs 56-61, wherein the corticosteroid is dexamethasone or betamethasone or a combination of dexamethasone and betamethasone.
63. A corticosteroid for use according to any one of the paragraphs 56-62, wherein the corticosteroid is dexamethasone and wherein the first dose is 4-20 mg and the second dose is 4-25 mg.
64. A corticosteroid for use according to paragraph 63, wherein the first dose is 10-12 mg and the second dose is 20 mg.
65. A corticosteroid for use according to any one of the paragraphs 56-62, wherein the corticosteroid is betamethasone and wherein the first dose is 3.2-16 mg and the second dose is 3.2-20 mg.
66. A corticosteroid for use according to paragraph 65, wherein the first dose is 8-9.6 mg and the second dose is 16 mg.
67. A corticosteroid for use according to claim any one of the paragraphs 56-66, wherein the dosing regimen further comprises administration of an antihistamine 10 minutes-24 hours prior to start of administration of the therapeutic antibody molecule.
68. A corticosteroid for use according to any one of the paragraphs 56-67, wherein the therapeutic antibody is an Fc receptor binding antibody.
69. A corticosteroid for use according to any one of the paragraphs 56-68, wherein the therapeutic antibody is an anti-FcγRIIB antibody.
70. A corticosteroid for use according to paragraph 69, wherein the anti-FcγRIIB antibody is the antibody having a light chain with SEQ ID No: 1 and a heavy chain with SEQ ID No: 2.
71. A therapeutic antibody molecule for use in the treatment of cancer, wherein the therapeutic antibody molecule has been predicted to be associated with a tolerability issue in connection with intravenous administration to a human using the method of any one of the paragraphs 44-55 and/or wherein the subcutaneous route of administration of therapeutic antibody molecule to a human has been predicted to prevent or mitigate the tolerability issue that otherwise would be associated with intravenous administration of the therapeutic antibody molecule to a human using the method of paragraph 51, or any one of the paragraphs 53-55 when referring to paragraph 51,
   and wherein the therapeutic antibody is formulated for subcutaneous administration.
72. A therapeutic antibody molecule for use according to paragraph 71, wherein the therapeutic antibody is an anti-FcγRIIB antibody.
73. A therapeutic antibody molecule for use according to paragraph 72, wherein the anti-FcγRIIB antibody is the antibody having a light chain with SEQ ID No: 1 and a heavy chain with SEQ ID No: 2.
74. A modified format of a therapeutic antibody molecule for use in the treatment of cancer, wherein the therapeutic antibody molecule has been predicted to be associated with a tolerability issue in connection with intravenous administration to a human using the method of any one of the paragraphs 44-55 and/or wherein administration of the therapeutic antibody molecule in the modified format to a human has been predicted to prevent or mitigate the tolerability issue that otherwise would be associated with intravenous administration of the therapeutic antibody molecule to a human using the method of paragraph 52, or any one of the paragraph 53-55 when referring to paragraph 52, and wherein the therapeutic antibody molecule is an Fc receptor binding antibody and the modified format is an antibody having the same Fv variable sequence but having impaired or abrogated FcγR binding compared with the therapeutic antibody molecule.
75. A modified format of a therapeutic antibody molecule for use according to paragraph 74, wherein the therapeutic antibody is an anti-FcγRIIB antibody.
76. A modified format of a therapeutic antibody molecule for use according to paragraph 75, wherein the modified format of the anti-FcγRIIB antibody is the antibody having a light chain with SEQ ID No: 1 and a heavy chain with SEQ ID No: 295.
77. A method for preventing or mitigating a tolerability issue in connection with intravenous administration of a therapeutic antibody molecule to a subject comprising a corticosteroid dosing regimen,
   wherein the therapeutic antibody molecule has been predicted to be associated with a tolerability issue in connection with intravenous administration to a human using the method of any one of the paragraphs 44-56, and/or wherein pre-treatment with the corticosteroid combination with administration of the therapeutic antibody molecule to a human has been predicted to prevent or mitigate the tolerability issue that otherwise would be associated with intravenous administration of the therapeutic antibody molecule to a human using the method of any one of the paragraphs 48-50, or any one of the paragraphs 53-56 when referring to any one of the paragraphs 48-50,
   and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).
78. A method according to paragraph 77, wherein the first dose is given 6-36 hours prior to start of administration of the therapeutic antibody molecule and the second dose is given 15-120 minutes prior to start of administration of the therapeutic antibody molecule.
79. A method according to paragraph 77 or 78, wherein the first dose is given 16-24 hours prior to start of administration of the therapeutic antibody molecule.
80. A method according to any one of the paragraphs 77-79, wherein the second dose is given 30-60 minutes prior to start of administration of the therapeutic antibody molecule.
81. A method according to any one of the paragraphs 77-80, wherein the dosing regimen comprises administration of the at least two doses of the corticosteroid prior to each infusion of the antibody during the course of antibody therapy.
82. A method according to any one of the paragraphs 77-81, wherein the corticosteroid is dexamethasone or betamethasone or a combination of dexamethasone and betamethasone.
83. A method according to any one of the paragraphs 77-82, wherein the corticosteroid is dexamethasone and wherein the first dose is 4-20 mg and the second dose is 4-25 mg.
84. A method according to paragraph 83, wherein the first dose is 10-12 mg and the second dose is 20 mg.
85. A method according to any one of the paragraphs 77-82, wherein the corticosteroid is betamethasone and wherein the first dose is 3.2-16 mg and the second dose is 3.2-20 mg.
86. A method according to paragraph 85, wherein the first dose is 8-9.6 mg and the second dose is 16 mg.
87. A method according to claim any one of the paragraphs 77-86, wherein the dosing regimen further comprises administration of an antihistamine 10 minutes-24 hours prior to start of administration of the therapeutic antibody molecule.
88. A method according to any one of the paragraphs 76-86, wherein the antibody is an Fc receptor binding antibody.
89. A method according to any one of the paragraphs 77-88, wherein the antibody is an anti-FcγRIIB antibody.
90. A method according to paragraph 89, wherein the anti-FcγRIIB antibody is the antibody having a light chain with SEQ ID No: 1 and a heavy chain with SEQ ID No: 2.
91. A method for treatment of cancer comprising subcutaneous administration of a therapeutically active amount of a therapeutic antibody molecule which has been predicted to be associated with a tolerability issue in connection with intravenous administration to a human using the method of any one of the paragraphs 44-56 and/or wherein the subcutaneous route of administration of therapeutic antibody molecule to a human has been predicted to prevent or mitigate the tolerability issue that otherwise would be associated with intravenous administration of the therapeutic antibody molecule to a human using the method of paragraph 51, or any one of the paragraphs 53-56 when referring to paragraph 51.
92. A method according to paragraph 91, wherein the antibody is an anti-FcγRIIB antibody.
93. A method according to paragraph 92, wherein the anti-FcγRIIB antibody is the antibody having a light chain with SEQ ID No: 1 and a heavy chain with SEQ ID No: 2.
94. A method for treatment of cancer comprising administration of a therapeutically active amount of a modified format of a therapeutic antibody, wherein the therapeutic antibody molecule has been predicted to be associated with a tolerability issue in connection with intravenous administration to a human using the method of any one of the paragraphs 44-56 and/or wherein administration of the therapeutic antibody molecule in the modified format to a human has been predicted to prevent or mitigate the tolerability issue that otherwise would be associated with intravenous administration of the therapeutic antibody molecule to a human using the method of paragraph 52, or any one of the paragraphs 53-56 when referring to paragraph 52,
   and wherein the therapeutic antibody molecule is an Fc receptor binding antibody and the modified format is an antibody having the same Fv variable sequence but having impaired or abrogated FcγR binding compared with the therapeutic antibody molecule.
95. A method according to paragraph 94, wherein the antibody is an anti-FcγRIIB antibody.
96. A method according to paragraph 95, wherein the anti-FcγRIIB antibody is the antibody having a light chain with SEQ ID No: 1 and a heavy chain with SEQ ID No: 2 with a N297Q mutation in the heavy chain.
97. A therapeutic antibody molecule for use in the treatment of cancer, an autoimmune disease, an inflammatory disease, an immunological disease, and/or an infectious disease, wherein the therapeutic antibody molecule is an anti-FcγRIIB antibody, and wherein the therapeutic antibody molecule is formulated for subcutaneous administration.
98. Use of a therapeutic antibody molecule in the manufacture of a medicament for use in the treatment of cancer, an autoimmune disease, an inflammatory disease, an immunological disease, and/or an infectious disease, wherein the therapeutic antibody molecule is an anti-FcγRIIB antibody having a light chain with SEQ ID No: 1 and a heavy chain with SEQ ID No: 2, and wherein the medicament is formulated for subcutaneous administration.
99. A pharmaceutical formulation comprising a therapeutic antibody molecule, wherein the therapeutic antibody molecule is an anti-FcγRIIB antibody having a light chain with SEQ ID No: 1 and a heavy chain with SEQ ID No: 2, and wherein the pharmaceutical formulation comprises a pharmaceutically acceptable diluent or excipient, and is formulated for subcutaneous administration.
100. A therapeutic antibody molecule for use according to paragraph 97, use of a therapeutic antibody molecule according to paragraph 98, or a pharmaceutical formulation according to paragraph 99, wherein the therapeutic antibody is an Fc receptor binding antibody.
101. A therapeutic antibody molecule for use according to paragraph 97 or 100, use of a therapeutic antibody molecule according to paragraph 98 or 100, or a pharmaceutical formulation according to paragraph 99 or 100, wherein the therapeutic antibody is an anti-FcγRIIB antibody.
102. A therapeutic antibody molecule for use according to paragraph 101, use of a therapeutic antibody molecule according to paragraph 101, or a pharmaceutical formulation according to paragraph 101, wherein the therapeutic antibody has a light chain with SEQ ID No: 1 and a heavy chain with SEQ ID No: 2.
103. A therapeutic antibody molecule for use according to paragraph 101 or 102, use of a therapeutic antibody molecule according to paragraph 101 or 102, or a pharmaceutical formulation according to paragraph 101 or 102, for treatment of cancer.
104. The pharmaceutical formulation according to any one of the paragraphs 99-103, wherein the therapeutic antibody is present at a concentration of between about 90 mg/mL and about 220 mg/mL.
105. The pharmaceutical formulation according to any one of the paragraphs 99-104, further comprising between about 5 mM and about 20 mM acetate, and/or between about 50 mM and about 250 mM NaCl, and/or about 0.05% Polysorbate 20, and/or wherein the pharmaceutical formulation is at a pH of between about pH 5.0 and about pH 5.8.
106. The pharmaceutical formulation according to any one of the paragraphs 99-105, wherein the formulation comprises:
   the therapeutic antibody at a concentration of 150 mg/mL;
   5-5 mM acetate;
   110 mM NaCl;
   0.05% (w/v) Polysorbate 20; and
   wherein the formulation is at a pH 5.8.
107. A method for the treatment of cancer, an autoimmune disease, an inflammatory disease, an immunological disease, and/or an infectious disease in a subject, the method comprising the step of administering to the subject a therapeutic antibody molecule, wherein the therapeutic antibody molecule is an Fc receptor binding antibody, and wherein the therapeutic antibody molecule is formulated for subcutaneous administration.
108. The method of paragraph 107, wherein the Fc receptor binding antibody is an anti-FcγRIIB antibody.
109. The method of paragraph 107 or 108, wherein the Fc receptor binding antibody is an anti-FcγRIIB antibody having a light chain with SEQ ID No: 1 and a heavy chain with SEQ ID No: 2.
110. A method for the treatment of cancer, an autoimmune disease, an inflammatory disease, an immunological disease, and/or an infectious disease in a subject, the method comprising the step of subcutaneously administering to the subject a pharmaceutical formulation as defined in any one of the paragraphs 99-106.
111. The method of paragraph 109 or 110 for the treatment of cancer.

1. A method for predicting if a therapeutic antibody molecule binding specifically to a human target will be associated with a tolerability issue in connection with intravenous administration to a human, comprising the following step:
(i) intravenous or intraperitoneal administration of the therapeutic antibody molecule, if cross-reactive with murine target, or a surrogate antibody, to a mouse and observation of the mouse during a period following immediately after the administration of the therapeutic or surrogate antibody, wherein a display of the macroscopic symptoms isolation and decreased activity during the period followed by restoration of the state of the mouse to the normal state is an indication that the intravenous administration of the therapeutic antibody molecule to a human will be associated with a tolerability issue,
and/or for predicting if a prophylactic or therapeutic treatment, an altered administration route and/or a modification of the therapeutic antibody molecule can prevent or mitigate a tolerability issue associated with intravenous administration to a human of a therapeutic antibody molecule binding specifically to a human target,
comprising the following step(s) in addition to (i) as set out above:
(ii) administration of a prophylactic or therapeutic agent to a mouse in conjunction with intravenous or intraperitoneal administration of the therapeutic or surrogate antibody to a mouse, and observation of the mouse during a period following immediately after the administration of the therapeutic or surrogate antibody, wherein a decreased display of the macroscopic symptoms compared to the macroscopic symptoms displayed by the mouse in (i) or no display of the macroscopic symptoms during the period is an indication that pre-treatment with the prophylactic or therapeutic agent in combination with administration of the therapeutic antibody molecule to a human can prevent or mitigate the tolerability issue that otherwise would be associated with intravenous administration of the therapeutic antibody molecule to a human;
(iii) administration of the therapeutic or surrogate antibody to a mouse by a route of administration other than intravenous or intraperitoneal administration, and observation of the mouse during a period following immediately after the administration of the therapeutic or surrogate antibody, wherein a decreased display of the macroscopic symptoms compared to the macroscopic symptoms displayed by the mouse in (i) or no display of the macroscopic symptoms during the period is an indication that the other route of administration can be used for administration of the therapeutic antibody molecule to a human to prevent or mitigate the tolerability issue that would be associated with intravenous administration of the therapeutic antibody molecule to a human; and/or
(iv) intravenous or intraperitoneal administration of a modified format of the therapeutic or surrogate antibody to a mouse by a route of administration other than intravenous or intraperitoneal administration, and observation of the mouse during a period following immediately after the administration of the modified therapeutic or surrogate antibody, wherein a decreased display of the macroscopic symptoms compared to the macroscopic symptoms displayed by the mouse in (i) or no display of the macroscopic symptoms during the period is an indication that administration of the therapeutic antibody molecule in the modified format to a human can be used to prevent or mitigate the tolerability issue that would be associated with intravenous administration of the therapeutic antibody molecule to a human.

2. A method according to claim 1, wherein a display in (i) of 1-3 additional macroscopic symptoms selected from impaired balance, piloerection, and hunching followed by un-natural body posture during the period in (i) where after the state of the mouse is restored to the normal state further strengthens the indication that the intravenous administration of the therapeutic antibody molecule to the human will be associated with a tolerability issue.

3. A method according to claim 1, wherein the period during which the macroscopic symptoms are displayed in (i) starts 5-10 minutes after administration of the therapeutic or surrogate antibody and ends 45-90 minutes after administration of the therapeutic or surrogate antibody, and wherein the observation period in (ii), (iii) and/or (iv) is of the same length.

4. A method according to claim 1, wherein at least one of the following additional parameters:
decreased blood pressure
decreased platelet count, and or
increased liver enzymes (AST/ALT).
observed during the period in (i) further strengthens the indication that the intravenous administration of the therapeutic antibody molecule to a human will be associated with a tolerability issue.

5. A method for predicting if a prophylactic or therapeutic treatment can prevent or mitigate a tolerability issue associated with intravenous administration to a human of a therapeutic antibody molecule binding specifically to a human target according to claim 1 comprising at least steps (i) and (ii), wherein pre-treatment is used in (ii) and wherein this pre-treatment is administration of a corticosteroid to the mouse prior to injection of the therapeutic or surrogate antibody.

6. A method according to claim 5, wherein the pre-treatment comprises two administrations of a corticosteroid, wherein one is given 10⁻⁴⁸ hours prior to administration of the therapeutic or surrogate antibody and the other is given 5 minutes-5 hours prior to administration of the therapeutic or surrogate antibody.

7. A method according to claim 6, wherein the corticosteroid is dexamethasone or betamethasone.

8. A method for predicting if an altered administration route can prevent or mitigate a tolerability issue associated with intravenous administration to a human of a therapeutic antibody molecule binding specifically to a human target according to claim 1 comprising at least steps (i) and (iii), wherein the route of administration used in (iii) is subcutaneous administration.

9. A method for predicting if a modification of the therapeutic antibody molecule can prevent or mitigate a tolerability issue associated with intravenous administration to a human of a therapeutic antibody molecule binding specifically to a human target according to claim 1 comprising at least steps (i) and (iv), wherein the modified format of the therapeutic or surrogate antibody used in (iv) is a modification that leads to decreased or abolished engagement of Fc receptors.

10. A method according to claim 1, wherein the human target is selected from the group consisting of FcγRIIB, FcγRIIA and CD40.

11. A method according to claim 10, wherein the therapeutic antibody molecule is a human anti-FcγRIIB antibody capable of binding a human FcγR via its Fc domain and wherein the mouse surrogate antibody is an anti-FcγRIIb antibody capable of binding a mouse FcγR via its Fc domain, such as wherein the therapeutic antibody molecule is a human anti-FcγRIIB IgG1 antibody and wherein the mouse surrogate antibody is an anti-FcγRIIb mIgG2a.

12. (canceled)

13. A method of preventing or mitigating a tolerability issue in connection with intravenous administration of a therapeutic antibody molecule to a subject comprising administering to said subject a corticosteroid,
wherein the therapeutic antibody molecule has been predicted to be associated with a tolerability issue in connection with intravenous administration to a human using the method of claim 1,
and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).

14. A method according to claim 13, wherein the therapeutic antibody is an Fc receptor binding antibody such as an anti-FcγRIIB antibody.

15. (canceled)

16. A method of preventing or mitigating a tolerability issue in connection with intravenous administration of a therapeutic antibody molecule to a subject comprising administering to the subject a corticosteroid,
wherein the therapeutic antibody molecule is an anti-FcγRIIB antibody,
and wherein the dosing regimen comprises administration of the corticosteroid to the subject in at least two doses prior to intravenous administration of the therapeutic antibody molecule, wherein one dose of the corticosteroid is administered 10-48 hours prior to start of the administration of therapeutic antibody molecule (“the first dose”) and one dose of the corticosteroid is administered 5 minutes-5 hours prior to the start of administration of the therapeutic antibody molecule (“the second dose”).

17. A method according to claim 13, wherein the first dose is given 6-36 hours prior to start of administration of the therapeutic antibody molecule and the second dose is given 15-120 minutes prior to start of administration of the therapeutic antibody molecule.

18. A method according to claim 13, wherein the first dose is given 16-24 hours prior to start of administration of the therapeutic antibody molecule, and/or the second dose is given 30-60 minutes prior to start of administration of the therapeutic antibody molecule.

19. (canceled)

20. A method according to claim 13, wherein the dosing regimen comprises administration of the at least two doses of the corticosteroid prior to each infusion of the antibody during the course of antibody therapy.

21. A method according to claim 13, wherein the corticosteroid is dexamethasone or betamethasone or a combination of dexamethasone and betamethasone.

22. A method according to claim 13, wherein the corticosteroid is dexamethasone and wherein the first dose is 4-20 mg and the second dose is 4-25 mg, or wherein the first dose is 10-12 mg and the second dose is 20 mg.

23. (canceled)

24. A method according to claim 13, wherein the corticosteroid is betamethasone and wherein the first dose is 3.2-16 mg and the second dose is 3.2-20 mg, or wherein the first dose is 8-9.6 mg and the second dose is 16 mg.

25. (canceled)

26. A method according to claim 13, wherein the dosing regimen further comprises administration of an antihistamine 10 minutes −24 hours prior to start of administration of the therapeutic antibody molecule.

27. A method according to claim 14, wherein the anti-FcγRIIB antibody is the antibody having a light chain with SEQ ID No: 1 and a heavy chain with SEQ ID No: 2.

28. A method of treating cancer in a subject comprising subcutaneously administering to the subject a therapeutic antibody molecule, wherein the therapeutic antibody molecule has been predicted to be associated with a tolerability issue in connection with intravenous administration to a human using the method of claim 1.

29. A method according to claim 28, wherein the therapeutic antibody is an anti-FcγRIIB antibody, such as wherein the anti-FcγRIIB antibody is the antibody having a light chain with SEQ ID No: 1 and a heavy chain with SEQ ID No: 2.

30. (canceled)

31. A method of treating cancer in a subject comprising administering to said subject a therapeutic antibody molecule, wherein the therapeutic antibody molecule has been predicted to be associated with a tolerability issue in connection with intravenous administration to a human using the method of claim 1,
and wherein the therapeutic antibody molecule is an Fc receptor binding antibody and the modified format is an antibody having the same Fv variable sequence but having impaired or abrogated FcγR binding compared with the therapeutic antibody molecule.

32. A method according to claim 31, wherein the therapeutic antibody is an anti-FcγRIIB antibody, optionally as wherein the modified format of the anti-FcγRIIB antibody is the antibody having a light chain with SEQ ID No: 1 and a heavy chain with SEQ ID No: 295.

33. (canceled)
